(19) United States
(12) Patent Application Publication
(10) Pub. No.: US 2016/0138038 A1
(43) Pub. Date: May 19, 2016

(54) Methods and Compositions for Improvement in Seed Yield

(71) Applicant: NORTH CAROLINA STATE UNIVERSITY, Raleigh, NC (US)

(72) Inventors: Heike Sederoff, Raleigh, NC (US); Rongda Qu, Holly Springs, NC (US); Jyoti Dalal Kajla, Cary, NC (US); Roopa Yalamanchili, Cary, NC (US)

(21) Appl. No.: 14/900,799

(22) PCT Filed: Jun. 20, 2014
(86) PCT No.: PCT/US2014/043407

§ 371 Date: Dec. 22, 2015

Related U.S. Application Data
(60) Provisional application No. 61/838,789, filed on Jun. 24, 2013.

(51) Int. Cl. C12N 15/82

(52) U.S. Cl. CPC C12N 15/8261

(57) ABSTRACT

This invention relates to methods for producing plants having an increased number of seeds and methods for producing plants having increased assimilate partitioning directed into fruits, seeds and/or other plant part (e.g., roots and/or tubers), and/or increased seed, root and/or tuber size, or any combination thereof.

### STATEMENT OF PRIORITY

This application is a continuation-in-part of International Application No. PCT/US2014/043407, filed Jun. 20, 2014, which claims the benefit of and priority from U.S. Provisional Patent Application Ser. No. 61/838,789, filed Jun. 24, 2013, and the entire contents of each of which are incorporated by reference herein.

### STATEMENT REGARDING ELECTRONIC FILING OF A SEQUENCE LISTING

A Sequence Listing in ASCII text format, submitted under 37 C.F.R. §1.821, entitled 5051-834WO_ST25.txt, 104,245 bytes in size, generated on Jun. 17, 2014 and filed via EFS-Web, is provided in lieu of a paper copy. This Sequence Listing is hereby incorporated herein by reference into the specification for its disclosures.

### STATEMENT OF GOVERNMENT SUPPORT

This invention was supported in part by funding provided under Grant No. DE-AR0000207 from the United States Department of Energy (DOE). The United States government has certain rights in this invention.

### FIELD OF THE INVENTION

The present invention relates to methods and compositions for modulating seed and fruit production as well as production of other plant parts.

### BACKGROUND

The performance of a plant, in terms of growth, development, and yield, is influenced by many factors including the plant's genotype and the impact of abiotic and biotic stresses to which the plant is exposed. The desire to improve the performance of plants in agricultural and horticultural settings has led to the development of selective breeding strategies to identify plants with desirable characteristics. Advances in genetic manipulation of plant germplasm have provided additional approaches to the improvement of plant traits. The genetic manipulation of a plant's germplasm can involve the identification and alteration of a single gene or multiple genes that influence one or more traits, thereby altering the plant's phenotype and, in some instances, its response to abiotic and biotic stresses. While many genes have been identified that influence important agricultural and horticultural traits, much remains to be learned. Thus, there is a need to identify additional means of improving the agricultural and horticultural traits of plants.

### SUMMARY OF THE INVENTION

This invention is directed to methods and compositions for modulating seed and fruit production.

Thus, in one aspect, the present invention provides a method for producing a plant having increased assimilate partitioning directed into fruits and/or seeds and/or other plant part(s) (e.g., root and/or tuber)) and/or increased seed, root and/or tuber size, comprising: modifying an endogenous cell wall invertase inhibitor (cwII) gene of a plant cell to produce a plant cell comprising in its genome said modified endogenous cell wall invertase inhibitor gene, wherein said modified endogenous cell wall invertase inhibitor gene encodes a polypeptide having reduced or no cell wall invertase inhibitor activity; and regenerating a plant from said plant cell, thereby producing a plant having increased assimilate partitioning into fruits and/or seeds and/or other plant part(s) (e.g., root and/or tuber)) and/or increased seed, tuber and/or root size of said plant as compared to a control.

In another embodiment, a method for producing a plant having increased assimilate partitioning directed into fruits, seeds and/or other plant part (e.g., roots and/or tubers), and/or increased seed, root and/or tuber size is provided, comprising introducing into a plant cell a heterologous polynucleotide operably linked to a promoter and encoding a suppressor of a cell wall invertase inhibitor (cwII); and regenerating a stably transformed plant from said plant cell, thereby producing a plant having increased assimilate partitioning directed into fruits, seeds and/or other plant part (e.g., roots and/or tubers), and/or increased seed, root and/or tuber size.

In another embodiment, method for producing a plant having an increased seed number and increased assimilate partitioning directed into fruits, seeds and/or other plant part (e.g., roots and/or tubers), and/or increased seed, root and/or tuber size is provided, the method comprising: introducing into a plant cell (a) a first heterologous polynucleotide operably linked to a promoter and encoding a suppressor of a cell wall invertase inhibitor and (b) a second heterologous polypeptide operably linked to a promoter and encoding polypeptides having the enzyme activity of glycolate dehydrogenase, to produce a stably transformed plant cell; and regenerating a stably transformed plant from said plant cell, wherein the regenerated transgenic plant comprises in its genome said first heterologous polynucleotide operably linked to a promoter and encoding a suppressor of a cell wall invertase inhibitor and said second heterologous polynucleotide operably linked to a promoter and encoding polypeptides having the enzyme activity of glycolate dehydrogenase, thereby producing a plant having an increased seed number and increased assimilate partitioning into fruits and/or seeds and/or other plant part(s) (e.g., root and/or tuber)) and/or increased seed, tuber and/or root size of said plant as compared to a control.

In still other aspects, the invention provides a method for producing a plant having an increased seed number and increased partitioning directed into fruits, seeds and/or other plant part (e.g., roots and/or tubers), and/or increased seed, root and/or tuber size, the method comprising: introducing into a plant cell (a) a first heterologous polynucleotide operably linked to a promoter and encoding a suppressor of a cell wall invertase inhibitor; (b) a second heterologous polypeptide operably linked to a promoter and encoding polypeptides having the enzyme activity of glycolate dehydrogenase, (c) a third heterologous polynucleotide operably linked to a promoter and encoding a polypeptide having the enzyme activity of tartronic semialdehyde reductase, and (d) a fourth heterologous polynucleotide operably linked to a promoter and encoding a polypeptide having the enzyme activity of glyoxylate carboligase, to produce a stably transformed plant cell; and regenerating a stably transformed plant from said plant cell, wherein the regenerated transgenic plant comprises in its genome said first heterologous polynucleotide operably linked to a promoter and encoding a suppressor of a cell wall invertase inhibitor, said second heterologous polynucleotide operably linked to a promoter and encoding polypeptides having the enzyme activity of glycolate dehydrogenase, said third heterologous polynucleotide operably linked to a promoter and encoding a polypeptide having the enzyme activity of tartronic semialdehyde reductase, and said fourth heterologous polynucleotide operably linked to a promoter and encoding a polypeptide having the enzyme activity of glyoxylate carboligase, thereby producing a plant having increased seed number and increased assimilate partitioning into fruits, seeds, roots and/or tubers and/or increased seed, tuber and/or root size in said transgenic plant as compared to a control.

In an additional aspect, the invention provides a method for producing a plant having increased seed number and partitioning directed into fruits, seeds and/or other plant part (e.g., roots and/or tubers), and/or increased seed, root and/or tuber size, comprising: modifying an endogenous cell wall invertase inhibitor (cwII) gene of a plant cell; and introducing into said plant cell a heterologous polynucleotide operably linked to a promoter and encoding polypeptides having the enzyme activity of glycolate dehydrogenase, to produce a plant cell comprising in its genome said heterologous polynucleotide operably linked to a promoter and encoding polypeptides having the enzyme activity of glycolate dehydrogenase and said modified endogenous cell wall invertase inhibitor gene, wherein said modified endogenous cell wall invertase inhibitor gene encodes a polypeptide having reduced or no cell wall invertase inhibitor activity, thereby producing a plant having increased seed number and increased assimilate partitioning directed into fruits, seeds and/or other plant part (e.g., roots and/or tubers), and/or increased seed, root and/or tuber size in said plant as compared to a control.

In a further aspect, a method for producing a plant having increased seed number and increased assimilate partitioning directed into fruits, seeds and/or other plant part (e.g., roots and/or tubers), and/or increased seed, root and/or tuber size is provided, the method comprising: modifying an endogenous cell wall invertase inhibitor (cwII) gene of a plant cell; and introducing into said plant cell (a) a first heterologous polynucleotide operably linked to a promoter and encoding polypeptides having the enzyme activity of glycolate dehydrogenase, (b) a second heterologous polynucleotide operably linked to a promoter and encoding a polypeptide having the enzyme activity of tartronic semialdehyde reductase, and (c) a third heterologous polynucleotide operably linked to a promoter and encoding a polypeptide having the enzyme activity of glyoxylate carboligase, to produce a plant cell comprising in its genome said first heterologous polynucleotide operably linked to a promoter and encoding polypeptides having the enzyme activity of glycolate dehydrogenase, said second heterologous polynucleotide operably linked to a promoter and encoding a polypeptide having the enzyme activity of tartronic semialdehyde reductase, said third heterologous polynucleotide operably linked to a promoter and encoding a polypeptide having the enzyme activity of glyoxylate carboligase and said modified endogenous cell wall invertase inhibitor gene, wherein said modified endogenous cell wall invertase inhibitor gene encodes a polypeptide having reduced or no cell wall invertase inhibitor activity, thereby producing a plant having increased seed number and increased assimilate partitioning directed into fruits, seeds and/or other plant part (e.g., roots and/or tubers), and/or increased seed, root and/or tuber size in said plant as compared to a control.

In a still further aspect, a method for producing a plant having an increased number of seeds is provided, comprising: introducing into a plant cell and/or plant part a first heterologous polynucleotide operably linked to a promoter and encoding polypeptides having the enzyme activity of glycolate dehydrogenase (GDH) and a second heterologous polynucleotide operably linked to a promoter and encoding a polypeptide having the activity of a CO₂ transporter (e.g., aquaporin) to produce a stably transformed plant cell and/or plant part expressing said first and second heterologous polynucleotides; and regenerating a stably transformed transgenic plant from said stably transformed plant cell and/or plant part, thereby producing a plant having an increased number of seeds.

In some aspects, the present invention provides a method for producing a plant having an increased number of seeds, comprising: introducing into a plant cell a first heterologous polynucleotide operably linked to a promoter and encoding polypeptides having the enzyme activity of glycolate dehydrogenase (GDH), a second heterologous polynucleotide operably linked to a promoter and encoding a polypeptide having the activity of a CO₂ transporter (e.g., aquaporin), a third heterologous polynucleotide operably linked to a promoter and encoding a polypeptide having the enzyme activity of a tartronic semialdehyde reductase and a fourth heterologous polynucleotide encoding a polypeptide having the enzyme activity of a glyoxylate carboligase to produce a stably transformed plant cell expressing said first, second, third and fourth heterologous polynucleotides; and regenerating a stably transformed transgenic plant from said stably transformed plant cell, thereby producing a plant having an increased number of seeds.

Another aspect of the invention provides a method for producing a plant having increased assimilate partitioning directed into fruits and/or seeds (and/or any other plant part) and increased seed size, comprising introducing into a plant cell a heterologous polynucleotide operably linked to a promoter and encoding a suppressor of a cell wall invertase inhibitor (cwII) and/or a second heterologous polynucleotide operably linked to a promoter and encoding a polypeptide having the enzyme activity of a cell wall invertase; and regenerating a stably transformed plant from said plant cell, thereby producing a plant having increased assimilate partitioning directed into fruits and/or seeds (and/or any other plant part) and increased seed size.

In a further aspect, the invention provides a method for producing a plant having an increased number of seeds, increased assimilate partitioning directed into fruits and/or seeds (and/or any other plant part) and increased seed size, comprising: introducing into a plant cell a first heterologous polynucleotide operably linked to a promoter and encoding polypeptides having the enzyme activity of glycolate dehydrogenase (GDH), a second heterologous polynucleotide operably linked to a promoter and encoding a polypeptide having the activity of a CO₂ transporter (e.g., aquaporin), and a third heterologous polynucleotide operably linked to a promoter and encoding a suppressor of an inhibitor of cell wall invertase (cwII) to produce a stably transformed plant cell expressing said heterologous polynucleotides; and regenerating a stably transformed plant from said stably transformed plant cell, thereby producing a stably transformed plant having an increased number of seeds, increased assimilate partitioning directed into fruits and/or seeds (and/or any other plant part) and increased seed size. In some aspects, a heterologous polynucleotide operably linked to a promoter and encoding a polypeptide having the enzyme activity of a cell wall invertase can be introduced into said plant cell instead of or in addition to the heterologous polynucleotide operably linked to a promoter and encoding a suppressor of an inhibitor of cell wall invertase.

In a still further aspect, the invention provides a method for producing a plant having an increased number of seeds, increased assimilate partitioning directed into fruits and/or seeds (and/or any other plant part) and increased seed size, comprising: introducing into a plant cell a first heterologous polynucleotide operably linked to a promoter and encoding polypeptides having the enzyme activity of glycolate dehydrogenase (GDH), a second heterologous polynucleotide operably linked to a promoter and encoding a polypeptide having the activity of a CO₂ transporter (e.g., aquaporin), a third heterologous polynucleotide operably linked to a promoter and encoding a suppressor of an inhibitor of cell wall invertase (cwII), a fourth heterologous polynucleotide operably linked to a promoter and encoding a polypeptide having the enzyme activity of a tartronic semialdehyde reductase and a fifth heterologous polynucleotide operably linked to a promoter and encoding a polypeptide having the enzyme activity of a glyoxylate carboligase to produce a stably transformed plant cell expressing said heterologous polynucleotides; and regenerating a stably transformed plant from said stably transformed plant cell, thereby producing a stably transformed plant having an increased number of seeds, increased assimilate partitioning directed into fruits and/or seeds (and/or any other plant part) and increased seed size. In some aspects, a heterologous polynucleotide operably linked to a promoter and encoding a polypeptide having the enzyme activity of a cell wall invertase can be introduced into said plant cell instead of or in addition to the heterologous polynucleotide operably linked to a promoter and encoding a suppressor of an inhibitor of cell wall invertase.

Further provided herein is a stably transformed plant, plant part or plant cell produced by any of the methods of this invention. In additional aspects, the present invention provides seeds and progeny plants produced from the plants of the invention as well as crops produced from the stably transformed plants of the invention. In other aspects, the invention provides as products produced from the transformed plants, plant parts and/or plant cells and progeny thereof of this invention.

The foregoing and other objects and aspects of the present invention are explained in detail in the drawings and specification set forth below.

### BRIEF DESCRIPTION OF THE DRAWINGS

FIG. 1 shows earlier floral induction in plants of transgenic line 69(DEF2+TG1) and 60(DEF2), as compared to WT at seven weeks of age. Plants were grown under short day conditions. WT=wild type; 69(DEF2+TG1)=GDH (GlcD, GlcE, GlcF), TSR and GCL transformant #69; 60(DEF2)=GDH transformant #60.

FIG. 2 shows the number of siliques produced per transgenic plant per week as compared to a wild type plant. Plants were grown under short day conditions. Number of plants per line=9. Error bars represent standard error. WT=wild type; 60(DEF2)=GDH transformant #60; 72(DEF2)=GDH transformant #72; 51(DEF2+TG1)=GDH, TSR and GCL transformant #51; 69(DEF2+TG1)=GDH, TSR and GCL transformant #69.

FIG. 3 shows an increase in the amount of seed harvested from transgenic plants expressing polynucleotides sequences encoding glycolate dehydrogenase and polynucleotide sequences encoding tartronic semialdehyde reductase and glyoxylate carboligase as compared to the WT. Seed per tube are all the seed harvested from one representative plant each. WT=wild type; 60(DEF2)=GDH transformant #60; 72(DEF2)=GDH transformant #72; 51(DEF2+TG1)=GDH, TSR and GCL transformant #51; 69(DEF2+TG1)=GDH, TSR and GCL transformant #69.

FIG. 4 shows an increase in the amount of seed harvested from transgenic plants expressing polynucleotides sequences encoding glycolate dehydrogenase and polynucleotide sequences encoding tartronic semialdehyde reductase and glyoxylate carboligase as compared to the WT. Seed per data point are averages of all the seed harvested from nine representative plant from each line. Error bars represent standard error. Stars represent significant difference using Student's t-test p<E-05. WT=wild type; 60(DEF2)=GDH transformant #60; 72(DEF2)=GDH transformant #72; 51(DEF2+TG1)=GDH, TSR and GCL transformant #51; 69(DEF2+TG1)=GDH, TSR and GCL transformant #69.

FIG. 5 shows the increase in weight of seeds in transgenic plants expressing aquaporin as compared to wild type plants. WT=wild type; AQP 9=aquaporin transformant #9; AQP 36=aquaporin transformant #36. Plants were exposed to a short duration of short day conditions (8 hrs light/16 hrs dark).

FIG. 6 shows the increase in weight of seeds in transgenic plants expressing aquaporin as compared to wild type plants. WT=wild type; Q 9=aquaporin transformant #9; Q 12=aquaporin transformant #12; Q32=aquaporin transformant #32; and Q36=aquaporin transformant #36. Plants were grown under normal conditions (12 hrs light/12 hrs dark)

FIGS. 7A-7B provide photographs showing the increased number of seeds in a transgenic plant expressing aquaporin as compared to a wild type plant. FIG. 7A shows the increased number of seeds collected from a transformant (AQP T3-9) expressing aquaporin as compared to the number of seeds collected from a wild type plant. FIG. 7B shows the increased number of seeds produced in a single silique of a transformant (AQP T3-32) expressing aquaporin as compared to the number of seeds collected from a wild type plant.

FIGS. 8A-8F show cell wall invertase 1 (cwII1) promoter activity in different tissues at different developmental stages. FIG. 8A shows expression in a transformant seedling at one day after germination (DAG). FIGS. 8B-8C show expression in seedlings at five DAG in a transformant (FIG. 8B) and a wild type (FIG. 8C). FIG. 8D shows expression in seedlings at five DAG from left-to-right in stem, first leaves, and root. Transformant plants at 13 DAG showing tissue preferred expression (FIG. 8E). Transformant seedlings at 29 DAG showing tissue preferred expression (FIG. 8F). The seedlings were transformed with a construct in which the GUS reporter gene was operably linked to the camelina cwII1 promoter.

FIG. 9 shows GUS transcript abundance in transformed seedlings at one, five, and twenty-nine days after germination. The seedlings were transformed with a construct in which the GUS reporter gene was operably linked to the camelina cwII1 promoter. Expression of the tubulin gene (Tub-1) is provided as a control.

FIGS. 10A-10C shows expression patterns of the cwII2 promoter in developing seed embryos. FIG. 10A shows expression in the root tip. FIG. 10B shows a radicle cross-section with expression in the steele. FIG. 10C shows a corresponding wild type cross-section with no GUS expression.

FIG. 11 shows reduced expression of cwII1 in T1 transformants. Expression of the tubulin gene (Tub-1) is provided as a control.

FIG. 12 shows the constructs with the endogenous promoters for these genes, pcwii-1 (P1) and pcwii-2 (P2), that were used to drive expression of isoform-specific artificial miRNA constructs Cwii-1 (S1), Cwii-2 (S2), or both (S3), either against their respective Cwii transcripts (P1-S1; P2-S2) or against both constructs (P1-S3).

FIGS. 13A-13C shows biomass and yields of camelina CWII repression lines. Transgenic T2 plants were grown to maturity. Transgenic plants of each genotype (P1-S1; P2-S2; P1-S3) had more vegetative biomass at maturity (FIG. 13A) and increased seed yield per plant (FIGS. 13B, 13C). The per-plant seed yield for transgene expressing lines was significantly higher than control plants (wt, empty vector; ev) for multiple independent lines (p≦0.05; 4≦n≦8).

FIG. 14 shows increased height in plants crossed between transgenic lines DEF2+TG1 (51) and P1-S1 (95) compared with either parent and WT plants at seven weeks of age. Plants were grown under long day conditions. WT=wild type; DEF2+TG1 (51)=GDH, TSR and GCL transformant #51; P1-S1 (95)=cell wall invertase inhibitor (cwII) RNAi.

FIG. 15 shows increased height in plants crossed between transgenic lines DEF2 (72) and P1-S1 (95) compared with either parent and WT plants at seven weeks of age. Plants were grown under long day conditions. WT=wild type; DEF2 (72)=GDH transformant #72; P1-S1 (95)=cwII RNAi.

FIG. 16 shows apparent CO₂ fixation in plants crossed between transgenic lines DEF2 (72) and P1-S1 (95) or DEF2+TG1 (51) and P1-S1 (95) compared with either parent and WT plants at seven weeks of age. N=3, error bars=standard error. Plants were grown under long day conditions. WT=wild type; DEF2 (72)=GDH transformant #72; DEF2+TG1 (51)=GDH, TSR and GCL transformant #51; P1-S1 (95)=cwII RNAi.

FIG. 17 shows increased height of plants crossed between transgenic lines DEF2 (72) and P1-S1 (95) or DEF2+TG1 (51) and P1-S1 (95) compared with either parent and WT plants at seven weeks of age. 4<N<24, error bars=standard error. Plants were grown under long day conditions. WT=wild type; DEF2 (72)=GDH transformant #72; DEF2+TG1 (51)=GDH, TSR and GCL transformant #51; P1-S1 (95)=cell wall invertase inhibitor RNAi.

FIG. 18 shows earlier floral development in plants crossed between transgenic lines DEF2 (72) and P1-S1 (95) or DEF2+TG1 (51) and P1-S1 (95) compared with the parents and WT plants at seven weeks of age. 4<N<24, error bars=standard error. Plants were grown under long day conditions. WT=wild type; DEF2 (72)=GDH transformant #72; DEF2+TG1 (51)=GDH, TSR and GCL transformant #51; P1-S1 (95)=cwII RNAi.

FIGS. 19A-19C show the constructs for introduction of show the constructs for introduction of the three subunits of Glycolate Dehydrogenase subunits GlcD, GlcE, and GlcF (DEF2) driven by independent promoters (Entcup4, 35S and ACT2) and containing the selection marker mCherry (FIG. 19A), the genes for TSR and GCL driven independently by the 35S promoter and containing the BAR genes as a selection marker (FIG. 19B) and the helix-loop-helix antisense repression construct for CWII1 driven by the endogenous CWII1-promoter and containing BAR as a selection marker (FIG. 19C). The constructs comprise origins, promoters, chloroplast transit peptides, and are codon optimized.

FIG. 20 shows the molecular analysis of wild type plants (WT), plants comprising the full bypass (DT), plants expressing RNAi targeted to the cell wall invertase inhibitor (P1S1), and plants comprising both the full bypass and P1S1 (C1, C2, and C3).

FIGS. 21A and 21B show average apparent photosynthetic rates (μmol*m⁻²*s⁻¹) in comparable leaves of 10 week old wt and transgenic plants grown either in under short of long day conditions (FIG. 21A) and leaf number of those plants at 10 weeks (FIG. 21B) under short day and long day conditions for wild type plants (WT), plants expressing the full bypass (Full Bypass), plants expressing RNAi targeted to the cell wall invertase inhibitor (P1S1) and plants expressing both the full bypass and P1S1 (Full Bypass X P1S1). Apparent rate of CO₂ fixation rate (A-value μmol/m²/s).

FIGS. 22A-22E shows height (in cm) at 10 weeks (FIG. 22A) and number of secondary shoots (FIG. 22B), six-week old plants grown under short day (FIG. 22C) and long day (FIG. 22D) conditions, and dry weight post harvest (without seed) (FIG. 22E) in wild type plants (WT), plants expressing the full bypass (Full Bypass), plants expressing RNAi targeted to the cell wall invertase inhibitor (P1S1) and plants expressing both the full bypass and P1S1 (Full Bypass X P1S1).

FIGS. 23A-23E show plant age at the time of flowering for plants grown under short day (FIG. 23A) and long day (FIG. 23B) conditions, pod production at week 10 (long day conditions) (FIG. 23C), the seed yield per plant for long day and short day grown plants (grams) (FIG. 23D) and number of seed produced per plant (FIG. 23E). Wild type plants (WT), plants expressing the full bypass (Full Bypass), plants expressing RNAi targeted to the cell wall invertase inhibitor (P1S1) and plants expressing both the full bypass and P1S1 (Full Bypass X P1S1).

FIGS. 24A-24E show average seed weight (mg) (FIG. 24A), average seed area (cm²) (FIG. 24B), volume for 100 seeds (FIG. 24C), average seed oil/protein moisture content (FIG. 24D) and seed total starch/carbohydrate assay (FIG. 24E). Wild type plants (WT), plants expressing the full bypass (Full Bypass), plants expressing RNAi targeted to the cell wall invertase inhibitor (P1S1) and plants expressing both the full bypass and P1S1 (Full Bypass X P1S1).

### DETAILED DESCRIPTION

This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof. Thus, for example, each of the embodiments described herein can be combined in various ways to produce further embodiments of the invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented. References to techniques employed herein are intended to refer to the techniques as commonly understood in the art, including variations on those techniques or substitutions of equivalent techniques that would be apparent to one of skill in the art.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The term “about,” as used herein when referring to a measurable value such as a dosage or time period and the like, refers to variations of ±20%, ±10%, 5%, ±1%, +0.5%, or even ±0.1% of the specified amount.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The terms “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

The terms “increase,” “increasing,” “increased,” “enhance,” “enhanced,” “enhancing,” and “enhancement” (and grammatical variations thereof), as used herein, describe an elevation in, for example, seed number, assimilate partitioning in to a seed or fruit (and/or any other plant part) and/or increased seed size (e.g., volume, weight, and the like) in a plant, plant part or plant cell. This increase can be observed by comparing the increase in the plant, plant part or plant cell transformed with, for example, one or more heterologous polynucleotides encoding polypeptides having the enzyme activity of glycolate dehydrogenase (GDH) and a heterologous polynucleotide encoding a CO₂ transporter (e.g., aquaporin) to the appropriate control (e.g., the same plant lacking (i.e., not transformed with) said heterologous polynucleotides). Thus, as used herein, the terms “increase,” “increasing,” “increased,” “enhance,” “enhanced,” “enhancing,” and “enhancement” (and grammatical variations thereof), and similar terms indicate an elevation of at least about, e.g., 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more, or any range therein, as compared to a control (e.g., a plant, plant part and/or plant cell that does not comprise said heterologous polynucleotides).

As used herein, the terms “reduce,” “reduced,” “reducing,” “reduction,” “diminish,” “suppress,” and “decrease” (and grammatical variations thereof), describe, for example, a decrease in cell wall invertase inhibitor expression or production in a plant, plant cell and/or plant part as compared to a control as described herein. Thus, as used herein, the terms “reduce,” “reduces,” “reduced,” “reduction,” “diminish,” “suppress,” and “decrease” and similar terms mean a decrease of at least about, e.g., 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 100%, and the like, or any range therein, as compared to a control (e.g., a plant, plant part and/or plant cell that does not comprise said heterologous polynucleotides).

The term “suppressor” as used herein, means a molecule (e.g., a polynucleotide or polypeptide) that when incorporated into a plant, plant part, or plant cell can “reduce,” “diminish,” “suppress,” and “decrease” the activity of another molecule (e.g., a polynucleotide or polypeptide) by at least about, e.g., 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 100%, and the like, or any range therein, as compared to a control (e.g., a plant, plant part and/or plant cell that does not comprise said suppressor). Thus, a heterologous polynucleotide encoding a suppressor of a cell wall invertase inhibitor (cwII) can comprise a polypeptide that suppresses cwII or it can encode a functional nucleic acid (e.g., RNAi) that suppresses cwII.

As used herein, the terms “express,” “expresses,” “expressed” or “expression,” and the like, with respect to a nucleotide sequence (e.g., RNA or DNA) indicates that the nucleotide sequence is transcribed and, optionally, translated. Thus, a nucleotide sequence may express a polypeptide of interest or a functional untranslated RNA. A “functional” RNA includes any untranslated RNA that has a biological function in a cell, e.g., regulation of gene expression. Such functional RNAs include but are not limited to RNAi (e.g., siRNA, shRNA, antisense RNA), miRNA, ribozymes, RNA aptamers, and the like.

As used herein, the term “overexpression” means increased expression over that in the control. In some embodiments, “overexpression” can include expression of a heterologous polynucleotide not normally expressed in an organism. In other embodiments, overexpression can include heterologous expression of an endogenous polynucleotide comprised in a heterologous expression cassette such that the amount of the endogenous polypeptide produced as a result of the endogenous polynucleotide comprised in the heterologous expression cassette is greater than is produced in the organism not transformed with said heterologous expression cassette. Thus, overexpression of a polynucleotide or polypeptide means an elevation of expression of at least about, e.g., 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more, or any range therein, as compared to a control (e.g., a plant, plant part and/or plant cell that does not comprise said heterologous polynucleotides of this invention). In some representative embodiments the polynucleotide encoding a CO₂ transporter (e.g., aquaporin) or encoding cell wall invertase can be overexpressed.

“Increased number of seeds,” “increasing the number of seeds,” “increased seed production,” “increasing seed production,” “increased seed yield” or “increasing seed yield” as used herein refers to the production of a greater number of seeds in a plant stably transformed with the heterologous polynucleotides of the invention (e.g., heterologous polynucleotides encoding polypeptides having the enzyme activity of glycolate dehydrogenase and the activity of a CO₂ transporter (e.g., aquaporin); and/or heterologous polynucleotides encoding polypeptides having the enzyme activity of glycolate dehydrogenase, the enzyme activity of tartronic semialdehyde reductase, the enzyme activity of glyoxylate carboligase and the activity of a CO₂ transporter) compared to a plant that is not transformed with the same heterologous polynucleotides. Seed number can be increased, for example, through increasing the number of seeds per seed bearing plant part (e.g., fruit, pod, loment, capsule, silique, follicle, achene, drupe, utricle, pome, and the like) and/or through increasing the number of seed bearing plant parts. In some embodiments, an increase in seed number produced by a stably transformed plant of this invention can be at least about, e.g., 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 100%, 105%, 110%, 120%, 125%, 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 296%, 300%, 310%, 320%, 330%, 340%, 350%, 375%, 400%, 450%, 500% or more, or any range therein, as compared to a control as described herein. In other embodiments, an increased number of seeds can be an increase of about 20% to about 200%, about 20% to about 250%, about 20% to about 300%, about 20% to about 350%, about 30% to about 200%, about 30% to about 250%, about 30% to about 300%, about 30% to about 350%, about 40% to about 200%, about 40% to about 250%, about 40% to about 300%, about 40% to about 350%, about 50% to about 200%, about 50% to about 250%, about 50% to about 300%, about 50% to about 350%, about 75% to about 200%, about 75% to about 250%, about 75% to about 300%, about 75% to about 350%, about 100% to about 200%, about 100% to about 250%, about 100% to about 300%, about 100% to about 350%, and the like, as compared to a control. In some particular embodiments, the increase in number of seeds can be about 120% to about 320%, about 150% to about 200%, about 150% to about 250%, about 150% to about 300%, about 150% to about 350%, and the like, as compared to a control.

“Increased assimilate partitioning directed into seeds and fruits” or “increasing assimilate partitioning directed into seeds and fruits,” “directed assimilate partitioning into seeds or fruits” refers to an increase in importing of sugars/assimilates into the fruit and seed/grain (e.g., phloem unloading into fruits/seeds/grains) of a plant that has been transformed or otherwise modified (e.g., a plant comprising a heterologous nucleotide sequence operably linked to a promoter and encoding a polypeptide having the enzyme activity of a cell wall invertase, a heterologous nucleotide sequence operably linked to a promoter and encoding a suppressor of a cell wall invertase inhibitor, or an endogenous cell wall invertase inhibitor gene modified to reduce or eliminate the cell wall invertase inhibitor activity of the polypeptide encoded by said gene) as compared to control plant (e.g., a plant not comprising said heterologous nucleotide sequences or modified endogenous cell wall invertase inhibitor gene). In some embodiments, the increased assimilate partitioning can be directed into other plant parts including but not limited to roots, modified (used here in the horticultural sense) roots (fusiform root, napiform root, conical root, etc.), leaves, stems, modified stems (tuber, rhizome, stolon, corm, etc.), and the like.

An increase in assimilate partitioning directed into seeds and fruits (or other plant parts (e.g., roots and/or tubers) of a stably transformed plant of this invention can be an increase of at least about, e.g., 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 100%, 150%, 200%, 300%, 400%, 500% or more, or any range therein, as compared to a control. The products of assimilate can be measured directly from tissue (e.g., glucose, sucrose, fructose), or downstream products (e.g., starch, oil, protein), and the like. In particular embodiments, an increase in assimilate partitioning directed into seeds and fruits (or other plant parts) of a stably transformed plant of this invention can be an increase of at least about, e.g., 2% to about 60%, 5% to about 55%, about 5% to about 50%, about 5% to about 60%, about 10% to about 50%, about 10% to about 55%, about 10% to about 60%, about 15% to about 45%, about 15% to about 50%, about 15% to about 55%, about 15% to about 60%, about 20% to about 40%, about 20% to about 45%, about 20% to about 50%, about 20% to about 55%, about 20% to about 60%, and the like.

As used herein, “increased seed size” refers to an increase in seed weight and/or seed volume. An increase in seed size or volume of seed produced by a stably transformed plant of this invention can be an increase of at least about, e.g., 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 100%, 150%, 200%, 300%, 400%, 500% or more, or any range therein, as compared to a control.

As used herein, “increased tuber size” refers to an increase in tuber weight and/or tuber volume. An increase in tuber size or volume produced by a stably transformed plant of this invention can be an increase of at least about, e.g., 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 100%, 150%, 200%, 300%, 400%, 500% or more, or any range therein, as compared to a control.

As used herein, “having the enzyme activity of” refers to a polypeptide having one or more enzymatic activities of said polypeptide. Thus, a polypeptide having the enzyme activities in accordance with this invention have at least about, e.g., 50%, 55%, 60%, 65%, 70%, 75%, 80%, 85%, 90%, 95%, 100%, 150%, 200%, 300%, 400%, 500% or more of one or more of the enzyme activities of said polypeptide.

Accordingly, the present invention is directed to compositions and methods for increasing the yield of seeds (e.g., the number of seeds) produced by a plant by introducing into the plant, plant cell and/or plant part a heterologous polynucleotide that encodes polypeptides having the activity of a glycolate dehydrogenase and a heterologous polynucleotide that encodes a polypeptide having the activity of a CO₂ transporter (e.g., aquaporin) as described herein. In other embodiments, the invention comprises introducing into the plant, plant part and/or plant cell a heterologous polynucleotide encoding a polypeptide operably linked to a promoter and encoding an enzyme having the activity of a cell wall invertase (cwI). In still other embodiments, the invention comprises introducing into the plant, plant part and/or plant cell a heterologous polynucleotide encoding a suppressor of a cell wall invertase inhibitor (cwII); for example, a functional nucleic acid, including but not limited to a functional nucleic acid that encodes a suppressor of a cell wall invertase inhibitor (cwII) (e.g., an RNAi construct that inhibits cell wall invertase inhibitor). In further embodiments, the invention comprises modifying an endogenous cell wall invertase inhibitor gene to reduce or eliminate the cell wall invertase inhibitor activity of the polypeptide encoded by said gene, which can be combined with the introduction of one or more heterologous polypeptides as described herein. In some embodiments, the invention further comprises introducing into the plant, plant part and/or plant cell additional heterologous polynucleotides encoding additional useful polypeptides or functional nucleic acids. For example, in some embodiments, additional useful polypeptides can include polypeptides having the enzyme activity of a glycolate dehydrogenase (GDH), a polypeptide having the enzyme activity of a tartronic semialdehyde reductase (TSR) and a polypeptide having the enzyme activity of a glyoxylate carboligase (GCL). Expression of one or more (e.g., 1, 2, 3, 4, 5, 6, 7, 0.8 and the like) of said polynucleotides in said stably transformed plant (or plant regenerated from said stably transformed plant part and/or plant produces a stably transformed plant having increased seed number, increased assimilate (e.g., sucrose) partitioning directed into the seeds and fruits (and/or other plant parts (e.g., tubers and/or roots)) and/or increased seed and/or tuber size as compared to a plant not comprising said one or more heterologous polynucleotides.

In any of the embodiments described herein, one or more of said polynucleotides can be introduced into a plant, plant part and/or plant part. Thus, one or more polynucleotides encoding a particular polypeptide as described herein can be introduced into a plant, and/or one or more polynucleotides encoding different polypeptides as described herein can be introduced into a plant in any combination.

Thus, a first aspect of the present invention provides a method for producing a plant having an increased number of seeds, comprising: introducing into a plant cell a first heterologous polynucleotide operably linked to a promoter and encoding polypeptides having the enzyme activity of glycolate dehydrogenase and a second heterologous polynucleotide operably linked to a promoter and encoding a polypeptide having the activity of a CO₂ transporter (e.g., aquaporin) to produce a stably transformed plant cell expressing said first and second heterologous polynucleotides; and regenerating a stably transformed transgenic plant from said stably transformed plant cell, wherein the regenerated transgenic plant comprises in its genome the first heterologous polynucleotide operably linked to a promoter and encoding polypeptides having the enzyme activity of glycolate dehydrogenase and the second heterologous polynucleotide operably linked to a promoter and encoding a polypeptide having the activity of a CO₂ transporter, thereby producing a stably transformed plant having an increased number of seeds compared to a control plant not comprising in its genome said first and second heterologous polynucleotides. The polypeptides having the enzyme activity of glycolate dehydrogenase can comprise, consist essentially of or consist of three proteins, GlcD, GlcE, GlcF, that together provide the glycolate dehydrogenase activity. Thus, in some embodiments, the heterologous polynucleotide operably linked to a promoter and encoding polypeptides having the enzyme activity of glycolate dehydrogenase comprises, consist essentially of, or consist of sequences encoding three proteins that can be operably linked to separate promoters or to a single promoter. In a representative embodiment, the heterologous polynucleotide encoding polypeptides having the enzyme activity of glycolate dehydrogenase comprises, consist essentially of or consist of sequences encoding GlcD, GlcE, GlcF, each of which are operably linked to separate promoters.

A second aspect of the present invention provides a method for producing a plant having an increased number of seeds, comprising: introducing into a plant cell a first heterologous polynucleotide operably linked to a promoter and encoding a polypeptide having the enzyme activity of tartronic semialdehyde reductase, a second heterologous polynucleotide operably linked to a promoter and encoding a polypeptide having the enzyme activity of glyoxylate carboligase and a third heterologous polynucleotide operably linked to a promoter and encoding a polypeptide having the activity of a CO₂ transporter to produce a stably transformed plant cell expressing said first, second and third heterologous polynucleotides; and regenerating a stably transformed transgenic plant from said stably transformed plant cell, wherein the regenerated transgenic plant comprises in its genome the first heterologous polynucleotide operably linked to a promoter and encoding a polypeptide having the enzyme activity of tartronic semialdehyde reductase, the second heterologous polynucleotide operably linked to a promoter and encoding a polypeptide having the enzyme activity of glyoxylate carboligase, and the third heterologous polynucleotide operably linked to a promoter and encoding a polypeptide having the activity of a CO₂ transporter, thereby producing a stably transformed plant having an increased number of seeds compared to a control plant not comprising in its genome said first, second and third heterologous polynucleotides.

In a third aspect, the present invention provides a method for producing a plant having an increased number of seeds, comprising: introducing into a plant cell a first heterologous polynucleotide operably linked to a promoter and encoding polypeptides having the enzyme activity of glycolate dehydrogenase, a second heterologous polynucleotide operably linked to a promoter and encoding a polypeptide having the activity of a CO₂ transporter (e.g., aquaporin), a third heterologous polynucleotide operably linked to a promoter and encoding a polypeptide having the enzyme activity of a tartronic semialdehyde reductase and a fourth heterologous polynucleotide operably linked to a promoter and encoding a polypeptide having the enzyme activity of a glyoxylate carboligase to produce a stably transformed plant cell expressing said first, second, third and fourth heterologous polynucleotides; and regenerating a stably transformed transgenic plant from said stably transformed plant cell, wherein the regenerated transgenic plant comprises in its genome the first heterologous polynucleotide encoding polypeptides having the enzyme activity of glycolate dehydrogenase, the second heterologous polynucleotide encoding a polypeptide having the activity of a CO₂ transporter, the third heterologous polynucleotide encoding a polypeptide having the enzyme activity of a tartronic semialdehyde reductase and the fourth heterologous polynucleotide encoding a polypeptide having the enzyme activity of a glyoxylate carboligase, thereby producing a stably transformed plant having an increased number of seeds compared to a control plant not comprising in its genome said first, second, third and fourth heterologous polynucleotides.

In some embodiments, the production of an increased number of seeds by said stably transformed plant as described herein occurs under non-stress conditions. In other embodiments, the production of an increased number of seeds by said stably transformed plant as described herein occurs under non-stress short day conditions. In particular embodiments, the production of an increased number of seeds by said stably transformed plant as described herein occurs under non-drought conditions.

A heterologous polynucleotide encoding polypeptides having the enzyme activity of glycolate dehydrogenase can be any heterologous polynucleotide that encodes a polypeptide having the enzyme activity of glycolate dehydrogenase. In some embodiments, the glycolate dehydrogenase is from E. coli and comprises three subunits, glcD, glcE, glcF, which when transformed in to a plant function as a glycolate dehydrogenase (e.g., NCBI Accession Nos: NC_000913.2; GI:49175990; EGW88309, GI:345356102 (glcD polypeptide); YP_026191.1, 49176295 (glcE polypeptide); EFF11610.1, GI:291469119 (glcF polypeptide), respectively)). The GDH subunits can be introduced into a plant separately or as a single construct. In a representative embodiment, the GDH are subunits operably linked to separate promoters and introduced into a plant in a single construct.

Thus, in representative embodiments, a heterologous polynucleotide encoding polypeptides having the enzyme activity of glycolate dehydrogenase useful with this invention can comprise, consist essentially of or consist of a nucleotide sequence of SEQ ID NO:1; SEQ ID NO:3 and/or SEQ ID NO:5 or a nucleotide sequence having substantial identity to said nucleotide sequences of SEQ ID NO:1; SEQ ID NO:3 and/or SEQ ID NO:5. In other embodiments, an amino acid sequence of a glycolate dehydrogenase can optionally comprise, consist essentially of or consist of the amino acid sequence of SEQ ID NO:2, SEQ ID NO:4 and/or SEQ ID NO:6, or an amino acid sequence having substantial identity to said amino acid sequence of SEQ ID NO:2, SEQ ID NO:4 and/or SEQ ID NO:6.

In further embodiments, said E. coli sequences can be codon-optimized for expression in plants. For example, the E. coli sequences can be codon-optimized according to an Arabidopsis codon table or a codon table for any other plant. Additionally, some nucleotides can be changed in the E. coli DNA sequences to preclude one or more restriction sites from the sequence. Accordingly, in particular embodiments, a heterologous polynucleotide encoding polypeptides having the enzyme activity of glycolate dehydrogenase useful with this invention can comprise, consist essentially of or consist of a nucleotide sequence of SEQ ID NO:7, SEQ ID NO:8 and/or SEQ ID NO:9 or a nucleotide sequence having substantial identity to said nucleotide sequences of SEQ ID NO:7, SEQ ID NO:8 and/or SEQ ID NO:9, which encode for a glycolate dehydrogenase that can optionally comprise, consist essentially of or consist of the amino acid sequence of SEQ ID NO:2, SEQ ID NO:4 and/or SEQ ID NO:6, or an amino acid sequence having substantial identity to said amino acid sequence of SEQ ID NO:2, SEQ ID NO:4 and/or SEQ ID NO:6.

A heterologous polynucleotide encoding a polypeptide having the enzyme activity of glyoxylate carboligase can be any heterologous polynucleotide that encodes a polypeptide having the enzyme activity of glyoxylate carboligase. In some embodiments, a heterologous polynucleotide encoding a polypeptide having the enzyme activity of glyoxylate carboligase can be that of NCBI Accession No: NP_415040. In representative embodiments of the invention, a heterologous polypeptide encoding a polypeptide having the enzyme activity of a glyoxylate carboligase can comprise, consist essentially of or consist of the nucleotide sequence of SEQ ID NO:10, or a nucleotide sequence having substantial identity to said nucleotide sequence of SEQ ID NO:10, which encodes for a glyoxylate carboligase that can optionally comprise, consist essentially of or consist of the amino acid sequence of the amino acid sequence of SEQ ID NO:11, or an amino acid sequence having substantial identity to said amino acid sequence of SEQ ID NO:10.

A heterologous polynucleotide encoding a polypeptide having the enzyme activity of tartronic semialdehyde reductase can be any heterologous polynucleotide that encodes a polypeptide having the enzyme activity of tartronic semialdehyde reductase. In some embodiments, a heterologous polynucleotide encoding a polypeptide having the enzyme activity of glyoxylate carboligase can that of NCBI Accession No: ABV04967.1 (GI:157065712). In representative embodiments, the heterologous polypeptide encoding a polypeptide having the enzyme activity of a tartronic semialdehyde reductase can comprise, consist essentially of or consist of the nucleotide sequence of SEQ ID NO:12, which encodes the amino acid sequence of SEQ ID NO:13.

Aquaporin is a high affinity CO₂ transporter with high similarity to the human CO₂ pore (AQP1) has been identified in tobacco (NtAQP1, e.g., aquaporin) and shown to facilitate CO₂ membrane transport in plants (Uehlein et al. Nature 425(6959): 734-7 (2003); Uehlein et al. Plant Cell 20(3):648-57 (2008); Flexas et al. Plant J. 48(3):427-39 (2006)). In some embodiments, a heterologous polynucleotide encoding a polypeptide having the activity of a CO₂/bicarbonate transporter can be used. In some embodiments, a heterologous polynucleotide encoding a polypeptide having the activity of a CO₂ transporter is from a plant (including, but not limited to, a saltwater algae), an extremophile archea and/or extremophile bacteria (e.g. from the marine microalgae Dunaliella spp.; and/or Hydrogenobacter thermophilis).

In representative embodiments, a heterologous polynucleotide encoding a CO₂ transporter (e.g., aquaporin) can comprise, consist essentially of or consist of a nucleotide sequence of SEQ ID NO:14, SEQ ID NO:16, SEQ ID NO:18 and/or SEQ ID NO:20, or a nucleotide sequence having substantial identity to said nucleotide sequences of SEQ ID NO:14, SEQ ID NO:16, SEQ ID NO:18 and/or SEQ ID NO:20. In other embodiments, an amino acid sequence of an a CO₂ transporter can optionally comprise, consist essentially of or consist of the amino acid sequence of SEQ ID NO:15, SEQ ID NO:17, SEQ ID NO:19 and/or SEQ ID NO:21, or an amino acid sequence having substantial identity to said nucleotide sequences of the amino acid sequence of SEQ ID NO:15, SEQ ID NO:17, SEQ ID NO:19 and/or SEQ ID NO:21.

In some embodiments, the heterologous polynucleotide encoding said a CO₂ transporter (e.g., aquaporin) is constitutively expressed, thereby overriding any endogenous developmental and/or tissue specific a CO₂ transporter expression in the plant, plant part and/or plant cell (See, e.g., Lian et al., Plant Cell Physiol 45: 481-489 (2004), Sade et al., New Phytol 181: 651-661 (2009), Sade et al., Plant Phys. 152:245-254 (2010)).

In further aspects of the invention, a method for producing a plant having increased assimilate (e.g., sucrose) partitioning directed into fruits and/or seeds of a plant and increased seed size is provided, the method comprising modifying the plant's endogenous cell wall invertase inhibitor gene to reduce or eliminate the cell wall invertase inhibitor activity of said modified gene, expressing in the plant a suppressor of an inhibitor of cell wall invertase (cwII) and/or expressing or overexpressing a cell wall invertase (cwI) in the plant. The export of sugars occurs from photosynthesizing mesophyll cells through the cell wall into the phloem/companion cell complex which carries sugars via mass flow to non-photosynthetic tissues. Phloem unloading occurs either via the cell wall (apoplasm) or via plasmodesmata (Koch, K. Curr Opin Plant Biol. 7(3):235-46 (2004); Ward et al. Intl. Rev. Cytol.—a Survey of Cell Biol. 178:41-71 (1998)). Export and import through the apoplasm are controlled by the activity of cell wall invertase (cwI), which hydrolyzes sucrose into glucose and fructose and is regulated by a specific inhibitor protein (cwII) (Ruan et al. Molecular Plant, 3(6):942-955 (2010); Greiner et al. Plant Physiol. 116(2):733-42 (1998)). Two general approaches have been used to modify sucrose flux: overexpression of cwI or repression of its inhibitor protein cwII (Wang et al. Nature Genetics. 40(11):1370-1374 (2008); Sonnewald et al. Plant J. 1(1):95-106 (1991); von Schaewen et al. Embo J. 9(10):3033-44 (1990); Zanor, M. I., et al. Plant Physiology 150(3):1204-1218 (2009); Jin et al. Plant Cell. 21(7):2072-89 (2009); Greiner et al. Nat Biotechnol. 17(7):708-11 (1999)).

As used herein, “modifying the plant's endogenous cell wall invertase inhibitor gene to reduce or eliminate the cell wall invertase inhibitor activity of said modified gene” includes not only the production of a cell wall invertase inhibitor polypeptide having reduced or no cell wall invertase inhibitor activity but also includes modification of the cell wall invertase inhibitor gene such that no cell wall invertase inhibitor polypeptide is produced.

In general, low cwI activity increases sucrose export from the source tissue, and high cwI activity increases sucrose unloading into fruits and seeds/grains. Quantitative trait loci analysis for fruit size in tomato (Lin5), and grain size in rice (GIF1) and maize (MN1) identified mutations in cell-wall invertases that led to reduction in its activity in pedicel/fruit tissues (Wang et al. Nature Genetics. 40(11):1370-1374 (2008); Fridman et al. Science 305(5691):1786-1789 (2004); Cheng et al. Plant Cell. 8(6):971-983 (1996)) as key regulators for phloem unloading and therefore determinants of seed and fruit size. Fruit-specific suppression of the cell wall invertase inhibitor (CwII) in tomato and rice led to increases in net seed/grain weight of 22% and 10%, respectively (Wang et al. Nature Genetics. 40(11):1370-1374 (2008); Jin et al. Plant Cell. 21(7):2072-89 (2009)).

Cell wall invertase inhibitors (cwII) are small peptides, with molecular masses (Mr) ranging from 15 to 23 kD, and may be localized to either the cell wall or vacuole (Krausgrill et al., Plant Journal 13(2): 275-280 (1998); Greiner et al. Plant Physiol. 116(2):733-42 (1998) Greiner et al. Australian Journal of Plant Physiology 27(9): 807-814 (2000). The functionality of these inhibitors has been determined largely by in vitro assays of their recombinant proteins (e.g., Greiner et al. Plant Physiol. 116(2):733-42 (1998); Bate et al., Plant Physiology 134 (1): 246-254 (2004). Cell wall and vacuolar invertases are highly stable proteins due to the presence of glycans, and as a result their activity may be highly dependent on posttranslational regulation by its inhibitory protein (Greiner et al., Australian Journal of Plant Physiology 27(9): 807-814 2000; Hothorn et al., Plant Cell 16 (12): 3437-3447(2004); Rausch and Greiner, Biochim Biophys Acta 1696(2):253-61 (2004)). Sequence comparisons can be done using known invertase inhibitors (Hothorn et al. Proc Natl Acad Sci USA. 107(40):17427-32 (2010)).

Methods for developing antisense silencing constructs or inhibitors generally are well known in the art. Thus, for example, for the purpose of silencing an inhibitor of cell wall invertase (cwII) of interest, the nucleotide sequence of the cwII of interest can be identified by sequence homology to known cwIIs using techniques that are standard in the art (See, e.g., Jin et al. Plant Cell 21:2072-2089 (2009)). Based on the nucleotide sequence of the cwII of interest, antisense nucleotide sequences can be prepared. Thus, for example, a cwII from Camelina sativa can be used to prepare RNAi for silencing an inhibitor of camelina cell wall invertase (e.g., SEQ ID NO:25 and/or SEQ ID NO:26). Once a cell wall invertase inhibitor has been identified homologous nucleotide sequences of cwII from a plant of interest can be readily identified using methods known in the art for identifying homologous nucleotide sequences.

In other embodiments, the activity of one or more cell wall invertase inhibitors can be repressed by knocking out the endogenous cwII genes using methods known in the art. Thus, as an alternative to silencing endogenous cwII through the introduction of a heterologous nucleotide sequence encoding a functional nucleic acid (e.g., RNAi, antisense, amiRNA), endogenous cwII of a plant can be modified to be non-functional (i.e., knocked-out) or to have reduced activity using art known methods using, for example, Zinc finger nuclease (ZFN) technology (see, e.g., Urnov et al. Genome editing with engineered zinc finger nucleases. Nature Reviews 11:636-646 (2010)); Transcription Activator-Like Effector Nuclease (TALEN) technology (see, e.g., Miller, J. C. et al. A TALE nuclease architecture for efficient genome editing. Nat. Biotechnol. 29, 143-148 (2011); and Christian, M. et al. Targeting DNA double-strand breaks with TAL effector nucleases. Genetics 186, 757-761 (2010)); the CRISPR/Cas system (SEE, E. G., Jiang, W., Bikard, D., Cox, D., Zhang, F. & Marraffini, L. A. RNA-guided editing of bacterial genomes using CRISPR-Cas systems. Nat. Biotechnol. 31, 233-239 (2013)); and engineered meganucleases technology (see, e.g., Antunes et al. Targeted DNA excision in Arabidopsis by a re-engineered homing endonuclease. BMC Biotechnology 12:86 (2012)). As would be understood by the skilled artisan, such methods can be readily applied to the polynucleotides/genes described herein, including, but not limited to, polynucleotides/genes encoding endogenous cell wall invertase and polynucleotides/genes encoding endogenous cell wall invertase inhibitor to alter the activity of the encoded peptide (i.e., overexpress endogenous cell wall invertase and reduce or eliminate the activity of endogenous cell wall invertase inhibitor).

Accordingly, some embodiments of the present invention provide methods for producing a plant having increased assimilate partitioning that is directed into fruit/seeds (or into any other plant part) and/or increased seed size by suppressing the plant's native cell wall invertase inhibitor using, for example, RNAi technology, or by modifying the native cell wall invertase inhibitor gene by, for example, genome editing or mutation, so that the activity of the native cell wall invertase inhibitor is reduced or eliminated.

In other embodiments, methods for increasing assimilate partitioning that is directed into fruit/seeds (and/or other plant part) of a plant and/or increasing seed size of a plant are provided via expression of a heterologous cell wall invertase and/or overexpression of a plant's native cell wall invertase, or any combination of overexpression of a plant's native cell wall invertase, expression of a heterologous cell wall invertase, and suppression of a cell wall invertase inhibitor, and/or modification of the native cell wall invertase inhibitor gene to reduce the cell wall invertase inhibitor activity of said gene. In representative embodiments, a native/endogenous cell wall invertase gene can be modified to overexpress in a plant. In further representative embodiments, a native/endogenous cell wall invertase inhibitor gene can be modified to reduce or eliminate the cell wall invertase inhibitor activity of the polypeptide encoded by said gene.

Thus, in some embodiments, the present invention provides a method for producing a plant having increased assimilate partitioning directed into fruits and/or seeds (and/or other plant part(s) (e.g., roots and/or tubers)) and/or increased seed and/or tuber or root size comprising: introducing into a plant cell a heterologous polynucleotide operably linked to a promoter and encoding a suppressor of a cell wall invertase inhibitor (cwII) to produce a stably transformed plant cell comprising in its genome said heterologous polynucleotide encoding a suppressor of a cell wall invertase inhibitor; and regenerating a stably transformed plant from said plant cell, thereby producing a stably transformed plant having increased assimilate partitioning into fruits and/or seeds and/or increased seed size as compared to a control (e.g., a plant not stably transformed with said heterologous polynucleotide operably linked to a promoter and encoding a suppressor of a cell wall invertase inhibitor).

In some embodiments, the suppressor of the inhibitor of cell wall invertase can be an RNAi. An exemplary RNAi suppressor of cell wall invertase inhibitor can be a sequence-specific inverted repeat (sense-intron-antisense). In representative embodiments, an RNAi useful with this invention for inhibition of cell wall invertase can comprise, consist essentially of or consist of the nucleotide sequence of SEQ ID NO:22, SEQ ID NO:23, and/or SEQ ID NO:24, or a nucleotide sequence having substantial identity to said sequences SEQ ID NO:22, SEQ ID NO:23, and/or SEQ ID NO:24, any fragment of SEQ ID NO:22, SEQ ID NO:23, and/or SEQ ID NO:24 capable of inhibiting cell wall invertase (e.g., a fragment comprising 10, 20, 30, 40, 50, 60, 70, 80, 90, 100, 150, 20, 250, 300, 350, 400, 450, 500, 550, 600, 650, 700, 750, 800, 850, 900, 950, 1000, 1100, 1200, 1300, 1400, 1500, 1600, 1700, 1800, 1900, 2000 nucleotides, and the like and any range therein of SEQ ID NO:22, SEQ ID NO:23, and/or SEQ ID NO:24).

In particular embodiments, a polynucleotide encoding a suppressor of a cell wall invertase inhibitor (e.g., cwII RNAi) can be operably linked to endogenous camelina promoters of the cell wall invertase inhibitors (e.g., SEQ ID NO:27, SEQ ID NO:28).

The present invention further provides methods for producing a plant having increased assimilate partitioning directed into fruits and/or seeds (and/or other plant part(s) (e.g., roots and tubers)) and/or increasing seed and/or tuber and/or root size, further comprising introducing a heterologous nucleotide sequence operably linked to a promoter and encoding a polypeptide having the enzyme activity of a cell wall invertase.

In representative embodiments, a heterologous polynucleotide encoding a polypeptide having the enzyme activity of a cell wall invertase can comprise, consist essentially of or consist of a nucleotide sequence of SEQ ID NO:45, SEQ ID NO:47, SEQ ID NO:49, SEQ ID NO:51 and/or SEQ ID NO:53, or a nucleotide sequence having substantial identity to said nucleotide sequences of SEQ ID NO:45, SEQ ID NO:47, SEQ ID NO:49, SEQ ID NO:51 and/or SEQ ID NO:53. In other embodiments, an amino acid sequence of a cell wall invertase can optionally comprise, consist essentially of or consist of the amino acid sequence of SEQ ID NO:46, SEQ ID NO:48; SEQ ID NO:50, SEQ ID NO:52, and/or SEQ ID NO:54, or an amino acid sequence having substantial identity to said nucleotide sequences of the amino acid sequence of SEQ ID NO:46, SEQ ID NO:48; SEQ ID NO:50, SEQ ID NO:52, and/or SEQ ID NO:54.

Thus, in some embodiments, the present invention further provides a method for producing a plant having increased assimilate partitioning directed into fruits and/or seeds (and/or other plant part(s) (e.g., root and/or tuber) as described herein) and/or increased seed and/or tuber and/or root size, the method comprising introducing into a plant cell a heterologous polynucleotide operably linked to a promoter and encoding a polypeptide having the enzyme activity of a cell wall invertase (cwI) to produce a stably transformed plant cell; and regenerating a stably transformed plant from said plant cell, wherein the regenerated transgenic plant comprises in its genome said heterologous polynucleotide operably linked to a promoter and encoding a polypeptide having the enzyme activity of a cell wall invertase, thereby producing a stable transgenic plant having increased assimilate partitioning directed into fruits and/or seeds (and/or other plant parts (e.g., roots and/or tubers)) and/or increased seed and/or tuber size as compared to a control (e.g., the same plant not stably transformed with said heterologous polynucleotide operably linked to a promoter and encoding a cell wall invertase). In particular embodiments, a cwI polynuceotide can be operably linked to one or more of the promoters of the cell wall invertase inhibitors (e.g., SEQ ID NO:30, SEQ ID NO:31) from camelina. In the alternative or in addition to introducing a heterologous polynucleotide encoding a cell wall invertase, an endogenous cell wall invertase gene of a plant can be modified to be overexpressed.

In further embodiments, a method for producing a plant having increased assimilate partitioning directed into fruits and/or seeds (and/or other plant part (e.g., root and/or tuber)) and/or increased seed size is provided, the method comprising introducing into a plant cell a first heterologous polynucleotide operably linked to a promoter and encoding a polypeptide having the enzyme activity of a cell wall invertase (cwI) and a second heterologous polynucleotide operably linked to a promoter and encoding a suppressor of an inhibitor of cell wall invertase to produce a stably transformed plant cell; and regenerating a stably transformed plant from said plant cell, wherein the regenerated transgenic plant comprises in its genome said first heterologous polynucleotide operably linked to a promoter and encoding a polypeptide having the enzyme activity of a cell wall invertase and said second heterologous polynucleotide operably linked to a promoter and encoding a suppressor of a cell wall invertase inhibitor, thereby producing a stably transformed plant having increased assimilate partitioning directed into fruits and/or seeds (and/or other plant parts (e.g., roots and/or tubers)) and/or increased seed and/or tuber size as compared to a control (e.g., the same plant not stably transformed with said first and second heterologous polynucleotides).

In still further embodiments, a cell wall invertase inhibitor can be suppressed directly through the use of genome editing techniques such as Zinc finger nucleases (ZFNs), Transcription Activator-Like Effector Nucleases (TALENs), the CRISPR/Cas system, and engineered meganucleases. As would be understood by the skilled artisan, other methods both known and later developed can be used for this purpose as well.

Accordingly, in some embodiments, a method for producing a plant having increased assimilate partitioning directed into fruits and/or seeds (and/or other plant part(s) (e.g., root and/or tuber size)) and/or increased seed, root and/or tuber size is provided, comprising: modifying an endogenous cell wall invertase inhibitor (cwII) gene of a plant cell to produce a plant cell comprising in its genome said modified endogenous cell wall invertase inhibitor gene, wherein said modified endogenous cell wall invertase inhibitor gene encodes a polypeptide having reduced or no cell wall invertase inhibitor activity; and regenerating a plant from said plant cell, thereby producing a plant having increased assimilate partitioning into fruits and/or seeds and/or increased seed size of said plant as compared to a control (e.g., the same plant but which does not comprise in its genome said modified cell wall invertase inhibitor gene).

In further embodiments, a method for producing a plant having increased assimilate partitioning directed into fruits and/or seeds (and/or other plant part(s) (e.g., root and/or tuber size)) and/or increased seed, root and/or tuber size is provided, the method comprising: modifying an endogenous cell wall invertase inhibitor (cwII) gene of a plant cell; and introducing into said plant cell a heterologous polynucleotide operably linked to a promoter and encoding a polypeptide having the enzyme activity of a cell wall invertase (cwI), to produce a plant cell comprising in its genome said heterologous polynucleotide operably linked to a promoter and encoding a polypeptide having the enzyme activity of a cell wall invertase and said modified endogenous cell wall invertase inhibitor gene, wherein said modified endogenous cell wall invertase inhibitor gene encodes a polypeptide having reduced or no cell wall invertase inhibitor activity, thereby producing a plant having increased assimilate partitioning directed into fruits and/or seeds (and/or other plant parts (e.g., roots and/or tubers)) and/or increased seed and/or tuber size as compared to a control (e.g., the same plant but which does not comprise in its genome said modified cell wall invertase inhibitor or said heterologous polypeptide).

Any method of modifying an endogenous nucleotide sequence or gene in a cell can be used to modify an endogenous cell wall invertase inhibitor gene in a plant cell to produce a plant cell having an endogenous cell wall invertase inhibitor gene encoding a polypeptide having reduced or no cell wall invertase inhibitor activity as described herein. In representative embodiments, the endogenous cell wall invertase inhibitor is modified using the CRISPR-Cas system. In some embodiments, the activity of the modified endogenous cell wall invertase inhibitor in a plant cell is reduced by at least about 10% to about 100%. Thus, in some embodiments, the activity of the modified endogenous cell wall invertase inhibitor in a plant cell is reduced by about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 100%, and the like, and any value or range therein.

Additional embodiments of the invention provide a method for producing a plant having an increased seed number and increased assimilate partitioning directed into fruits and/or seeds (and/or other plant part) and/or increased seed size, the method comprising: introducing into a plant cell a first heterologous polynucleotide operably linked to a promoter and encoding a suppressor of a cell wall invertase inhibitor and a second heterologous polypeptide operably linked to a promoter and encoding a CO₂ transporter (e.g., aquaporin) to produce a stably transformed plant cell; and regenerating a stably transformed plant from said plant cell, wherein the regenerated transgenic plant comprises in its genome said first heterologous polynucleotide operably linked to a promoter and encoding a suppressor of a cell wall invertase inhibitor and said second heterologous polynucleotide operably linked to a promoter and encoding a CO₂ transporter, thereby producing a plant having an increased seed number and increased assimilate partitioning directed into fruits and/or seeds (and/or other plant part) and/or increased seed size in said transgenic plant as compared to a control (e.g., a plant not stably transformed with said first and second heterologous polynucleotides).

In other embodiments, a method for producing a plant having an increased seed number and increased assimilate partitioning directed into fruits and/or seeds (and/or other plant part) and/or increased seed size in a plant is provided, the method comprising introducing into a plant cell a first heterologous polynucleotide operably linked to a promoter and encoding a polypeptide having the enzyme activity of a cell wall invertase and a second heterologous polypeptide operably linked to a promoter and encoding a CO₂ transporter (e.g., aquaporin) to produce a stably transformed plant cell; and regenerating a stably transformed plant from said plant cell, wherein the regenerated transgenic plant comprises in its genome the first heterologous polynucleotide operably linked to a promoter and encoding a polypeptide having the enzyme activity of a cell wall invertase and the second heterologous polypeptide operably linked to a promoter and encoding a CO₂ transporter, thereby producing a plant having increased seed number and increased assimilate partitioning directed into fruits and/or seeds (and/or other plant part) and/or increased seed size in said transformed plant as compared to a control (e.g., the same plant not stably transformed with said first and second heterologous polynucleotides).

In further embodiments, a method for producing a plant having an increased seed number and increased assimilate partitioning directed into fruits and/or seeds (and/or other plant part) and/or increased seed size in a plant is provided, the method comprising introducing into a plant cell a first heterologous polynucleotide operably linked to a promoter and encoding a polypeptide having the enzyme activity of a cell wall invertase (cwI), a second heterologous polynucleotide operably linked to a promoter and encoding a suppressor of an inhibitor of cell wall invertase (cwII) and a third heterologous polypeptide operably linked to a promoter and encoding a CO₂ transporter (e.g., aquaporin) to produce a stably transformed plant cell; and regenerating a stably transformed plant from said plant cell, wherein the regenerated transgenic plant comprises in its genome said first heterologous polynucleotide operably linked to a promoter and encoding a polypeptide having the enzyme activity of a cell wall invertase, said second heterologous polynucleotide operably linked to a promoter and encoding a suppressor of a cell wall invertase inhibitor and said third heterologous polypeptide operably linked to a promoter and encoding a CO₂ transporter, thereby producing a stably transformed plant having an increased seed number and increased assimilate partitioning directed into fruits and/or seeds (and/or other plant part) and/or increased seed size in said transgenic plant as compared to a control (e.g., the same plant not stably transformed with said first, second and third heterologous polynucleotides).

Additional embodiments of the invention provide a method for producing a plant having an increased seed number and increased assimilate partitioning directed into fruits and/or seeds (and/or other plant part) and/or increased seed size, the method comprising: introducing into a plant cell a first heterologous polynucleotide operably linked to a promoter and encoding a suppressor of a cell wall invertase inhibitor and a second heterologous polypeptide operably linked to a promoter and encoding polypeptides having the enzyme activity of glycolate dehydrogenase, to produce a stably transformed plant cell; and regenerating a stably transformed plant from said plant cell, wherein the regenerated transgenic plant comprises in its genome said first heterologous polynucleotide operably linked to a promoter and encoding a suppressor of a cell wall invertase inhibitor and said second heterologous polynucleotide operably linked to a promoter and encoding polypeptides having the enzyme activity of glycolate dehydrogenase, thereby producing a plant having an increased seed number and increased assimilate partitioning directed into fruits and/or seeds (and/or other plant part) and/or increased seed size in said transgenic plant as compared to a control (e.g., a plant not stably transformed with said first and second heterologous polynucleotides).

In other embodiments, a method for producing a plant having an increased seed number and increased assimilate partitioning directed into fruits and/or seeds (and/or other plant part) and/or increased seed size in a plant is provided, the method comprising introducing into a plant cell a first heterologous polynucleotide operably linked to a promoter and encoding a polypeptide having the enzyme activity of a cell wall invertase and a second heterologous polypeptide operably linked to a promoter and encoding polypeptides having the enzyme activity of glycolate dehydrogenase, to produce a stably transformed plant cell; and regenerating a stably transformed plant from said plant cell, wherein the regenerated transgenic plant comprises in its genome the first heterologous polynucleotide operably linked to a promoter and encoding a polypeptide having the enzyme activity of a cell wall invertase and the second heterologous polypeptide operably linked to a promoter and encoding polypeptides having the enzyme activity of glycolate dehydrogenase, thereby producing a plant having increased seed number and increased assimilate partitioning directed into fruits and/or seeds (and/or other plant part) and/or increased seed size in said transformed plant as compared to a control (e.g., the same plant not stably transformed with said first and second heterologous polynucleotides). In particular aspects, the polynucleotide encoding a polypeptide having the activity of a cell wall invertase is overexpressed in the plant. In an additional aspect, the method further comprises introducing a third heterologous polynucleotide operably linked to a promoter and encoding a polypeptide having the enzyme activity of tartronic semialdehyde reductase, and a fourth heterologous polynucleotide operably linked to a promoter and encoding a polypeptide having the enzyme activity of glyoxylate carboligase, to produce a stably transformed plant cell; and regenerating a stably transformed plant from said plant cell, wherein the regenerated transgenic plant comprises in its genome the first, second, third and fourth heterologous polynucleotides and has increased seed number, and increased assimilate partitioning directed into fruits and/or seeds (and/or other plant part) and/or increased seed size.

In further embodiments, a method for producing a plant having an increased seed number and increased assimilate partitioning directed into fruits and/or seeds (and/or other plant part) and/or increased seed size is provided, the method comprising introducing into a plant cell a first heterologous polynucleotide operably linked to a promoter and encoding a polypeptide having the enzyme activity of a cell wall invertase (cwI), a second heterologous polynucleotide operably linked to a promoter and encoding a suppressor of an inhibitor of cell wall invertase (cwII) and a third heterologous polypeptide operably linked to a promoter and encoding polypeptides having the enzyme activity of glycolate dehydrogenase to produce a stably transformed plant cell; and regenerating a stably transformed plant from said plant cell, wherein the regenerated transgenic plant comprises in its genome said first heterologous polynucleotide operably linked to a promoter and encoding a polypeptide having the enzyme activity of a cell wall invertase, said second heterologous polynucleotide operably linked to a promoter and encoding a suppressor of a cell wall invertase inhibitor and said third heterologous polypeptide operably linked to a promoter and encoding polypeptides having the enzyme activity of glycolate dehydrogenase, thereby producing a stably transformed plant having an increased seed number and increased assimilate partitioning directed into fruits and/or seeds (and/or other plant part) and/or increased seed size in said transgenic plant as compared to a control (e.g., the same plant not stably transformed with said first, second and third heterologous polynucleotides). In an additional aspect, the method further comprises introducing a fourth heterologous polynucleotide operably linked to a promoter and encoding a polypeptide having the enzyme activity of tartronic semialdehyde reductase, and a fifth heterologous polynucleotide operably linked to a promoter and encoding a polypeptide having the enzyme activity of glyoxylate carboligase, to produce a stably transformed plant cell; and regenerating a stably transformed plant from said plant cell, wherein the regenerated transgenic plant comprises in its genome the first, second, third, fourth and fifth heterologous polynucleotides and has increased seed number, and increased assimilate partitioning directed into fruits and/or seeds (and/or other plant part) and/or increased seed size.

In additional aspects, the present invention provides a method for producing a plant having an increased number of seeds and increased assimilate partitioning directed into fruits and/or seeds (and/or other plant part) and/or increased seed size in a plant, the method comprising introducing into a plant cell a first heterologous polynucleotide operably linked to a promoter and encoding polypeptides having the enzyme activity of glycolate dehydrogenase, a second heterologous polynucleotide operably linked to a promoter and encoding a polypeptide having the activity of a CO₂ transporter (e.g., aquaporin), and a third heterologous polynucleotide encoding a suppressor of an inhibitor of cell wall invertase to produce a stably transformed plant cell comprising in its genome the first heterologous polynucleotide operably linked to a promoter and encoding polypeptides having the enzyme activity of glycolate dehydrogenase, the second heterologous polynucleotide operably linked to a promoter and encoding a polypeptide having the activity of a CO₂ transporter (e.g., aquaporin) and the third heterologous polynucleotide operably linked to a promoter and encoding a suppressor of an inhibitor of cell wall invertase; and regenerating a stably transformed plant from said plant cell, thereby producing a stably transformed plant having an increased number of seeds and increased assimilate partitioning directed into fruits and/or seeds (and/or other plant part) and/or increased seed size in said transgenic plant as compared to a control (e.g., the same plant not stably transformed with said first, second and third heterologous polynucleotides).

In an additional embodiment, a method for producing a plant having an increased number of seeds and increased assimilate partitioning directed into fruits and/or seeds (and/or other plant part) and/or increased seed size in a plant is provided, the method comprising introducing into a plant cell a first heterologous polynucleotide operably linked to a promoter and encoding polypeptides having the enzyme activity of glycolate dehydrogenase, a second heterologous polynucleotide operably linked to a promoter and encoding a polypeptide having the activity of a CO₂ transporter (e.g., aquaporin), and a third heterologous polynucleotide operably linked to a promoter and encoding a polypeptide having the enzyme activity of a cell wall invertase to produce a stably transformed plant cell comprising in its genome the first heterologous polynucleotide encoding polypeptides operably linked to a promoter and having the enzyme activity of glycolate dehydrogenase, the second heterologous polynucleotide operably linked to a promoter and encoding a polypeptide having the activity of a CO₂ transporter and the third heterologous polynucleotide operably linked to a promoter and encoding a polypeptide having the enzyme activity of a cell wall invertase; and regenerating a stably transformed plant from said plant cell, thereby producing a stably transformed plant having an increased number of seeds and increased assimilate partitioning directed into fruits and/or seeds and/or increased seed size as compared to a control (e.g., the same plant not stably transformed with said first, second and third heterologous polynucleotides).

In another aspect, the present invention provides a method for producing a plant having an increased number of seeds and increased assimilate partitioning directed into fruits and/or seeds (and/or other plant part) and/or increased seed size in a plant, the method comprising introducing into a plant cell a first heterologous polynucleotide operably linked to a promoter and encoding a polypeptide having the enzyme activity of tartronic semialdehyde reductase, a second heterologous polynucleotide operably linked to a promoter and encoding a polypeptide having the enzyme activity of glyoxylate carboligase, a third heterologous polynucleotide operably linked to a promoter and encoding a polypeptide having the activity of a CO₂ transporter (e.g., aquaporin), and a fourth heterologous polynucleotide operably linked to a promoter and encoding a suppressor of an inhibitor of cell wall invertase to produce a stably transformed plant cell, wherein the regenerated transgenic plant comprises in its genome the first heterologous polynucleotide operably linked to a promoter and encoding a polypeptide having the enzyme activity of a tartronic semialdehyde reductase, the second heterologous polynucleotide operably linked to a promoter and encoding a polypeptide having the enzyme activity of a glyoxylate carboligase, the third heterologous polynucleotide operably linked to a promoter and encoding a polypeptide having the activity of a CO₂ transporter, and the fourth heterologous polynucleotide operably linked to a promoter and encoding a suppressor of an inhibitor of cell wall invertase, thereby producing a plant having an increased number of seeds and increased assimilate partitioning directed into fruits and/or seeds (and/or any other plant part) and/or increased seed size as compared to a control (e.g., the same plant not stably transformed with said first, second, third and fourth heterologous polynucleotides).

In a further aspect, the present invention provides a method for producing a plant having an increased number of seeds and increased assimilate partitioning directed into fruits and/or seeds (and/or other plant part) and/or increased seed size, the method comprising introducing into a plant cell a first heterologous polynucleotide operably linked to a promoter and encoding a polypeptide having the enzyme activity of tartronic semialdehyde reductase, a second heterologous polynucleotide operably linked to a promoter and encoding a polypeptide having the enzyme activity of glyoxylate carboligase, a third heterologous polynucleotide operably linked to a promoter and encoding a polypeptide having the activity of a CO₂ transporter (e.g., aquaporin), and a fourth heterologous polynucleotide operably linked to a promoter and encoding a polypeptide having the enzyme activity of a cell wall invertase to produce a stably transformed plant cell, wherein the regenerated transgenic plant comprises in its genome the first heterologous polynucleotide operably linked to a promoter and encoding a polypeptide having the enzyme activity of a tartronic semialdehyde reductase, the second heterologous polynucleotide operably linked to a promoter and encoding a polypeptide having the enzyme activity of a glyoxylate carboligase, the third heterologous polynucleotide operably linked to a promoter and encoding a polypeptide having the activity of a CO₂ transporter, and the fourth heterologous polynucleotide operably linked to a promoter and encoding a cell wall invertase, thereby producing a stably transformed plant having an increased number of seeds and increased assimilate partitioning directed into fruits and/or seeds and/or increased seed size in said transgenic plant as compared to a control (e.g., the same plant not stably transformed with said first, second, third and fourth heterologous polynucleotides).

In a still further embodiment, the present invention provides a method for producing a plant having an increased number of seeds and increased assimilate partitioning directed into fruits and/or seeds (and/or other plant part) and/or increased seed size, the method comprising introducing into a plant cell a first heterologous polynucleotide operably linked to a promoter and encoding polypeptides having the enzyme activity of glycolate dehydrogenase, a second heterologous polynucleotide operably linked to a promoter and encoding a polypeptide having the activity of a CO₂ transporter (e.g., aquaporin), a third heterologous polynucleotide operably linked to a promoter and encoding a polypeptide having the enzyme activity of a tartronic semialdehyde reductase, a fourth heterologous polynucleotide operably linked to a promoter and encoding a polypeptide having the enzyme activity of a glyoxylate carboligase, and a fifth heterologous polynucleotide operably linked to a promoter and encoding a suppressor of an inhibitor of cell wall invertase to produce a stably transformed plant cell; and regenerating a stably transformed plant from said plant cell, wherein the regenerated transgenic plant comprises in its genome the first heterologous polynucleotide operably linked to a promoter and encoding polypeptides having the enzyme activity of glycolate dehydrogenase, the second heterologous polynucleotide operably linked to a promoter and encoding a polypeptide having the activity of a CO₂ transporter, the third heterologous polynucleotide operably linked to a promoter and encoding a polypeptide having the enzyme activity of a tartronic semialdehyde reductase, the fourth heterologous polynucleotide operably linked to a promoter and encoding a polypeptide having the enzyme activity of a glyoxylate carboligase and the fifth heterologous polynucleotide operably linked to a promoter and encoding a suppressor of an inhibitor of cell wall invertase, thereby producing a plant having an increased number of seeds and increased assimilate partitioning directed into fruits and/or seeds (and/or any other plant part) and/or increased seed size in said transgenic plant as compared to a control (e.g., the same plant not stably transformed with said first, second, third, fourth and fifth heterologous polynucleotides). In some embodiments, the method further comprises introducing into said plant cell an additional heterologous polynucleotide, wherein the additional heterologous polynucleotide is operably linked to a promoter and encodes a cell wall invertase.

In an additional embodiment, the present invention provides a method for producing a plant having an increased number of seeds and increased assimilate partitioning directed into fruits and/or seeds (and/or other plant part) and/or increased seed size in a plant, the method comprising introducing into a plant cell a first heterologous polynucleotide operably linked to a promoter and encoding polypeptides having the enzyme activity of glycolate dehydrogenase, a second heterologous polynucleotide operably linked to a promoter and encoding a polypeptide having the activity of a CO₂ transporter (e.g., aquaporin), a third heterologous polynucleotide operably linked to a promoter and encoding a polypeptide having the enzyme activity of a tartronic semialdehyde reductase, a fourth heterologous polynucleotide operably linked to a promoter and encoding a polypeptide having the enzyme activity of a glyoxylate carboligase, and a fifth heterologous polynucleotide operably linked to a promoter and encoding a polypeptide having the enzyme activity of cell wall invertase to produce a stably transformed plant cell; and regenerating a stably transformed plant from said plant cell, wherein the regenerated transgenic plant comprises in its genome the first heterologous polynucleotide operably linked to a promoter and encoding polypeptides having the enzyme activity of glycolate dehydrogenase, the second heterologous polynucleotide operably linked to a promoter and encoding a polypeptide having the activity of a CO₂ transporter, the third heterologous polynucleotide operably linked to a promoter and encoding a polypeptide having the enzyme activity of a tartronic semialdehyde reductase, the fourth heterologous polynucleotide operably linked to a promoter and encoding a polypeptide having the enzyme activity of a glyoxylate carboligase and the fifth heterologous polynucleotide operably linked to a promoter and encoding a polypeptide having the enzyme activity of cell wall invertase, thereby producing a stably transformed plant having an increased number of seeds and increased assimilate partitioning directed into fruits and/or seeds (and/or any other plant part) and/or increased seed size in said transgenic plant as compared to a control (e.g., the same plant not stably transformed with said first, second, third, fourth and fifth heterologous polynucleotides).

Further embodiments provide a method for producing a plant having increased seed number and increased assimilate partitioning directed into fruits and/or seeds (and/or other plant part) and/or increased seed size, comprising: modifying an endogenous cell wall invertase inhibitor (cwII) gene of a plant cell; and introducing into said plant cell a heterologous polynucleotide operably linked to a promoter and encoding polypeptides having the enzyme activity of glycolate dehydrogenase, to produce a plant cell comprising in its genome said heterologous polynucleotide operably linked to a promoter and encoding polypeptides having the enzyme activity of glycolate dehydrogenase and said modified endogenous cell wall invertase inhibitor gene, wherein said modified endogenous cell wall invertase inhibitor gene encodes a polypeptide having reduced or no cell wall invertase inhibitor activity, thereby producing a plant having increased seed number and increased assimilate partitioning directed into fruits and/or seeds (and/or other plant part) and/or increased seed size in said plant as compared to a control (e.g., the same plant but which does not comprise in its genome said modified cell wall invertase inhibitor or said heterologous polynucleotide). In some embodiments, the method further comprises introducing into said plant cell one or more additional heterologous polynucleotides, wherein said additional heterologous polynucleotide are operably linked to one or more promoters and encode a CO₂ transporter and/or a polypeptide having the enzyme activity of a cell wall invertase (cwI). In representative embodiments, when present, the heterologous polynucleotides encoding a cell wall invertase (cwI) or a CO₂ transporter can each be overexpressed in said plant cell.

In other embodiments, a method for producing a plant having increased seed number and increased assimilate partitioning directed into fruits and/or seeds (and/or other plant part) and/or increased seed size is provided, the method comprising: modifying an endogenous cell wall invertase inhibitor (cwII) gene of a plant cell; and introducing into said plant cell a first heterologous polynucleotide operably linked to a promoter and encoding polypeptides having the enzyme activity of glycolate dehydrogenase, a second heterologous polynucleotide operably linked to a promoter and encoding a polypeptide having the enzyme activity of tartronic semialdehyde reductase, and a third heterologous polynucleotide operably linked to a promoter and encoding a polypeptide having the enzyme activity of glyoxylate carboligase, to produce a plant cell comprising in its genome said first heterologous polynucleotide operably linked to a promoter and encoding polypeptides having the enzyme activity of glycolate dehydrogenase, said second heterologous polynucleotide operably linked to a promoter and encoding a polypeptide having the enzyme activity of tartronic semialdehyde reductase, said third heterologous polynucleotide operably linked to a promoter and encoding a polypeptide having the enzyme activity of glyoxylate carboligase and said modified endogenous cell wall invertase inhibitor gene, wherein said modified endogenous cell wall invertase inhibitor gene encodes a polypeptide having reduced or no cell wall invertase inhibitor activity, thereby producing a plant having increased seed number and increased assimilate partitioning directed into fruits and/or seeds (and/or other plant part) and/or increased seed size in said plant as compared to a control (e.g., the same plant but which does not comprise in its genome a modified cell wall invertase inhibitor gene and said first, second and third heterologous polynucleotides). In some embodiments, the method further comprises introducing into said plant cell one or more additional heterologous polynucleotides, wherein said additional heterologous polynucleotide are operably linked to one or more promoters and encode a CO₂ transporter and/or a polypeptide having the enzyme activity of a cell wall invertase (cwI). In representative embodiments, when present, the heterologous polynucleotides encoding a cell wall invertase (cwI) or a CO₂ transporter can each be overexpressed in said plant cell.

A heterologous polynucleotide operably linked to a promoter and encoding a suppressor of a cell wall invertase inhibitor and/or a heterologous polynucleotide operably linked to a promoter and encoding a polypeptide having the enzyme activity of a cell wall invertase can be introduced (in any order) into a plant in any combination with one or more additional polynucleotides, including any of the heterologous polynucleotides encoding polypeptides having the enzyme activity of glycolate dehydrogenase, a polypeptide having the enzyme activity of a tartronic semialdehyde reductase and/or a polypeptide having the enzyme activity of a glyoxylate carboligase to increase the number of seeds and/or increase the assimilate partitioning directed into fruits and/or seeds and/or increase the seed size in a plant. Further, a method wherein an endogenous cell invertase inhibitor gene of a plant is modified can be combined (in any order, i.e., the modification of the endogenous cell wall invertase inhibitor gene can be done first, in between or after the introduction of one or more heterologous polynucleotides) with the introduction of one or more heterologous polynucleotides into said plant as described herein.

In each of the embodiments above, said polynucleotide operably linked to a promoter and encoding the polypeptide having the activity of a CO₂ transporter (e.g., aquaporin) and/or the heterologous polynucleotide operably linked to a promoter and encoding a polypeptide having the enzyme activity of a cell wall invertase can be overexpressed in the stably transformed plant.

In further embodiments, the heterologous polypeptides and heterologous polynucleotides useful with this invention as described herein (e.g., those encoding polypeptides having the activity of GDH, TSR, GCL, CO₂ transporter, cwI, cwII, or a suppressor of cwII) can be modified for use with this invention. For example, a native or wild type intergenic spacer sequence in a selected polynucleotide can be substituted with another known spacer or a synthetic spacer sequence. In some embodiments, a polynucleotide or gene can be modified to increase or decrease the activity of the encoded polypeptide.

Other modifications of polypeptides useful with this invention include amino acid substitutions (and the corresponding base pair changes in the respective polynucleotide encoding said polypeptide). Thus, in some embodiments, a polypeptide and/or polynucleotide sequence of the invention can be a conservatively modified variant. As used herein, “conservatively modified variant” refers to polypeptide and polynucleotide sequences containing individual substitutions, deletions or additions that alter, add or delete a single amino acid or nucleotide or a small percentage of amino acids or nucleotides in the sequence, where the alteration results in the substitution of an amino acid with a chemically similar amino acid. Conservative substitution tables providing functionally similar amino acids are well known in the art.

As used herein, a “conservatively modified variant” of a polypeptide is biologically active and therefore possesses the desired activity of the reference polypeptide. The variant can result from, for example, a genetic polymorphism or human manipulation. A biologically active variant of the reference polypeptide can have at least about, e.g., 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more sequence identity (e.g., about 30% to about 99% or more sequence identity and any range therein) to the amino acid sequence for the reference polypeptide as determined by sequence alignment programs and parameters described elsewhere herein. An active variant can differ from the reference polypeptide sequence by as few as 1-15 amino acid residues, as few as 1-10, such as 6-10, as few as 5, as few as 4, 3, 2, or even 1 amino acid residue.

Naturally occurring variants may exist within a population. Such variants can be identified by using well-known molecular biology techniques, such as the polymerase chain reaction (PCR), and hybridization as described below. Synthetically derived nucleotide sequences, for example, sequences generated by site-directed mutagenesis or PCR-mediated mutagenesis which still encode a polypeptide of the invention, are also included as variants. One or more nucleotide or amino acid substitutions, additions, or deletions can be introduced into a nucleotide or amino acid sequence disclosed herein, such that the substitutions, additions, or deletions are introduced into the encoded protein. The additions (insertions) or deletions (truncations) may be made at the N-terminal or C-terminal end of the native protein, or at one or more sites in the native protein. Similarly, a substitution of one or more nucleotides or amino acids may be made at one or more sites in the native polynucleotide or protein.

For example, conservative amino acid substitutions may be made at one or more predicted, preferably nonessential amino acid residues. A “nonessential” amino acid residue is a residue that can be altered from the wild-type sequence of a protein without altering the biological activity, whereas an “essential” amino acid is required for biological activity. A “conservative amino acid substitution” is one in which the amino acid residue is replaced with an amino acid residue with a similar side chain. Families of amino acid residues having similar side chains are known in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Such substitutions would not be made for conserved amino acid residues, or for amino acid residues residing within a conserved motif, where such residues are essential for protein activity.

In some embodiments, amino acid changes can be made to alter the catalytic activity of an enzyme. For example, amino acid substitutions can be made to a thermoactive enzyme that has little activity at room temperature (e.g., about 20° C. to about 50° C.) so as to increase activity at these temperatures. A comparison can be made between the thermoactive enzyme and a mesophilic homologue having activity at the desired temperatures. This can provide discrete differences in amino acids that can then be the focus of amino acid substitutions.

Thus, in some embodiments, amino acid sequence variants of a reference polypeptide can be prepared by mutating the nucleotide sequence encoding the polypeptide. The resulting mutants can be expressed recombinantly in plants, and screened for those that retain biological activity by assaying for the activity of the polypeptide (e.g., glycolate dehydrogenase, a CO₂ transporter, and the like) using standard assay techniques as described herein. Methods for mutagenesis and nucleotide sequence alterations are known in the art. See, e.g., Kunkel (1985) Proc. Natl. Acad. Sci. USA 82:488-492; Kunkel et al. (1987) Methods in Enzymol. 154:367-382; and Techniques in Molecular Biology (Walker & Gaastra eds., MacMillan Publishing Co. 1983) and the references cited therein; as well as U.S. Pat. No. 4,873,192. Clearly, the mutations made in the DNA encoding the variant must not disrupt the reading frame and preferably will not create complementary regions that could produce secondary mRNA structure. See, EP Patent Application Publication No. 75,444. Guidance as to appropriate amino acid substitutions that do not affect biological activity of the protein of interest may be found in the model of Dayhoff et al. (1978) Atlas of Protein Sequence and Structure (National Biomedical Research Foundation, Washington, D.C.).

In some embodiments, deletions, insertions and substitutions in the polypeptides useful with this invention are not expected to produce radical changes in the characteristics of the polypeptide (e.g., the temperature at which the polypeptide is active). However, when it is difficult to predict the exact effect of the substitution, deletion or insertion in advance of doing so, one of skill in the art will appreciate that the effect can be evaluated by routine screening assays for the particular polypeptide activities (e.g., GDH, TSR, GCL, CO₂ transporter and the like) as described herein. Further, when so desired, deletions, insertions and substitutions and other modification can be made to the polypeptides useful with this invention in order to modify their activity as described herein.

In some embodiments, the compositions of the invention can comprise active fragments of the polypeptide. As used herein, “fragment” means a portion of the reference polypeptide that retains the polypeptide activity of GDH, TSR, GCL, cell wall invertase and/or a CO₂ transporter. A fragment also means a portion of a nucleic acid molecule encoding the reference polypeptide. An active fragment of the polypeptide can be prepared, for example, by isolating a portion of a polypeptide-encoding nucleic acid molecule that expresses the encoded fragment of the polypeptide (e.g., by recombinant expression in vitro), and assessing the activity of the fragment. Nucleic acid molecules encoding such fragments can be at least about, e.g., 150, 200, 250, 300, 350, 400, 450, 500, 550, 600, 650, 700, 800, 900, 1,000, 1,100, 1,200, 1,300, 1,400, 1,500, 1,600, 1,700, 1,800, 1,900, or 2000 contiguous nucleotides, or up to the number of nucleotides present in a full-length polypeptide-encoding nucleic acid molecule. As such, polypeptide fragments can be at least about, e.g., 50, 60, 70, 80, 90, 100, 125, 150, 175, 200, 225, 250, 275, 300, 325, 350, 375, 400, 425, 450, 475, 500, 525, 550, 575, 600, 625, 650, 675, or 700 contiguous amino acid residues, or up to the total number of amino acid residues present in the full-length polypeptide.

Methods for assaying the activities of glycolate dehydrogenase, tartronic semialdehyde reductase, glyoxylate carboligase, cell wall invertase, CO₂ transporter, suppressor of cwII and cwII are known in the art. Thus, for example, glycolate dehydrogenase activity can be assayed by monitoring the rate of the enzyme-dependent conversion of glycolate to glyoxylate as described by Lord et al (1972) Biochimica et Biophysica Acta 267:227-237. The activities of glyoxylate carboligase and tartronic semialdehyde reductase can be assayed in a combined assay monitoring the enzyme-dependent oxidation of NADH to NAD using glyoxylate as a substrate, as described by Gotto et al. (1961) Biochem J 81:273-281. Relative cwII activity can be determined by assaying for cell wall invertase (cwI) activity as described by Hothorn et al (2010) (Proceedings of the National Academy of Sciences 107(40): 17427-17432) or Tomlinson et al (2004) (J Exp. Bot. 55(406): 2291-2303). Cell wall invertase inhibition assays followed the protocol of Weil et al. (Planta 193:438-445 (1994)). Invertase preparations and Nt-inh1 protein preparations were mixed and incubated for 60 min at 37° C. in the absence or presence of 20 mm Suc. After this preincubation 20 mm Suc was also added to the minus-Suc sample, and the Glc released during a subsequent 60-min incubation at 37° C. was determined enzymatically.

In some embodiments, the heterologous polynucleotide encoding polypeptides having the enzyme activity of glycolate dehydrogenase, the heterologous polynucleotide encoding polypeptides having the activity of a CO₂ transporter (e.g., aquaporin), the heterologous polynucleotide encoding polypeptides having the enzyme activity of tartronic semialdehyde reductase, the heterologous polynucleotide encoding polypeptides having the enzyme activity of glyoxylate carboligase as well as any other heterologous polynucleotide encoding a polypeptide or functional nucleic acid of interest (e.g., a heterologous polynucleotide encoding a polypeptide having the enzyme activity of a cell wall invertase and/or heterologous polynucleotide encoding a suppressor of an inhibitor of cell wall invertase (e.g., RNAi)) can be comprised within one or more expression cassettes, in any combination. As used herein, “expression cassette” means a recombinant nucleic acid molecule comprising at least one polynucleotide sequence of interest (e.g., a heterologous polynucleotide encoding GDH, TSR, GCL, CO₂ transporter, a cell wall invertase (cwI) and/or a suppressor of an inhibitor of cell wall invertase (cwII), and the like), wherein said recombinant nucleic acid molecule is operably associated with at least a control sequence (e.g., a promoter). Thus, some embodiments of the invention provide expression cassettes designed to express a heterologous polynucleotide encoding polypeptides having the enzyme activity of glycolate dehydrogenase, a heterologous polynucleotide encoding a polypeptide having the activity of a CO₂ transporter, a heterologous polynucleotide encoding a polypeptide having the enzyme activity of a tartronic semialdehyde reductase, a heterologous polynucleotide encoding a polypeptide having the enzyme activity of a glyoxylate carboligase, a heterologous polynucleotide encoding a polypeptide having the enzyme activity of a cell wall invertase and/or a heterologous polynucleotide encoding a suppressor of an inhibitor of cell wall invertase.

An expression cassette comprising a recombinant nucleic acid molecule may be chimeric, meaning that at least one of its components is heterologous with respect to at least one of its other components. An expression cassette may also be one that is naturally occurring but has been obtained in a recombinant form useful for heterologous expression.

In some embodiments, the heterologous polynucleotide encoding the polypeptide having the enzyme activity of glycolate dehydrogenase, the heterologous polynucleotide encoding a polypeptide having the activity of a CO₂ transporter (e.g., aquaporin), the heterologous polynucleotide encoding a polypeptide having the enzyme activity of tartronic semialdehyde reductase, the heterologous polynucleotide encoding a polypeptide having the enzyme activity of glyoxylate carboligase, the heterologous polynucleotide encoding a polypeptide having the enzyme activity of a cell wall invertase and/or the heterologous polynucleotide encoding a suppressor of an inhibitor of cell wall invertase can be comprised in a single expression cassette or they can each be comprised in different expression cassettes, in any combination. Thus, for example, the heterologous polynucleotides encoding the polypeptide having the enzyme activity of glycolate dehydrogenase (e.g., the three subunits GlcD, GlcE, GlcF) can be introduced in a single expression cassette while the polynucleotide encoding a polypeptide having the enzyme activity of tartronic semialdehyde reductase, the polynucleotide encoding a polypeptide having the enzyme activity of glyoxylate carboligase and the polynucleotide encoding a polypeptide having the activity of a CO₂ transporter can be introduced together in a second expression cassette, and the heterologous polynucleotide encoding a polypeptide having the activity of a cell wall invertase and the heterologous polynucleotide encoding a suppressor of a cell wall invertase inhibitor can be introduced together in a third expression cassette.

In further embodiments, the heterologous polynucleotide encoding the polypeptide having the enzyme activity of glycolate dehydrogenase, the heterologous polynucleotide encoding a polypeptide having the enzyme activity of tartronic semialdehyde reductase, and the heterologous polynucleotide encoding a polypeptide having the enzyme activity of glyoxylate carboligase can be introduced into a plant on a single expression cassette and the heterologous polynucleotide encoding a polypeptide having the activity of a CO₂ transporter (e.g., aquaporin), the heterologous polynucleotide encoding a polypeptide having the activity of a cell wall invertase and the heterologous polynucleotide encoding a suppressor of a cell wall invertase inhibitor can be introduced together in a further expression cassette.

In a still further embodiment, the heterologous polynucleotide encoding the polypeptide having the enzyme activity of glycolate dehydrogenase and the heterologous polynucleotide encoding a polypeptide having the activity of a CO₂ transporter (e.g., aquaporin) can be introduced into a plant on a single expression cassette and the heterologous polynucleotide encoding a polypeptide having the enzyme activity of tartronic semialdehyde reductase, the heterologous polynucleotide encoding a polypeptide having the enzyme activity of glyoxylate carboligase, the heterologous polynucleotide encoding a polypeptide having the activity of a cell wall invertase and the heterologous polynucleotide encoding a suppressor of a cell wall invertase inhibitor can be introduced together in a further expression cassette.

In some embodiments, the expression cassettes comprising the heterologous polynucleotides can comprise one or more regulatory elements in addition to a promoter as described herein (e.g., enhancers, introns, translation leader sequences, translation termination sequences, and polyadenylation signal sequences).

Thus, as disclosed herein, the various polynucleotides of this invention (e.g., encoding GDH, TSR, GCL, a CO₂ transporter, cwI and/or cwII) can be comprised in one or more expression cassettes in almost any configuration in taking into consideration factors well known to those in art to be important in the construction of expression cassettes (for example, regulatory controls, targeting, tissue specific expression, size and interactions between the polynucleotides (e.g., the suppressor)).

When the heterologous polynucleotides are comprised within more than one expression cassette, said heterologous polynucleotide encoding a polypeptide having the enzyme activity of a glycolate dehydrogenase, said heterologous polynucleotide encoding a polypeptide having the activity of a CO₂ transporter, said heterologous polynucleotide encoding a polypeptide having the enzyme activity of a tartronic semialdehyde reductase, said heterologous polynucleotide encoding a polypeptide having the enzyme activity of a glyoxylate carboligase, said heterologous polynucleotide encoding a suppressor of a cell wall invertase inhibitor and/or said heterologous polynucleotide encoding a polypeptide having the enzyme activity of a cell wall invertase can be introduced into plants singly or more than one at a time using co-transformation methods as known in the art.

In some embodiments, said heterologous polynucleotide encoding a polypeptide having the enzyme activity of a glycolate dehydrogenase, said heterologous polynucleotide encoding a polypeptide having the enzyme activity of a tartronic semialdehyde reductase, said heterologous polynucleotide encoding a polypeptide having the enzyme activity of a glyoxylate carboligase, said heterologous polynucleotide encoding a polypeptide having the activity of a CO₂ transporter (e.g., aquaporin), said heterologous polynucleotide encoding a polypeptide having the enzyme activity of a cell wall invertase, and/or said heterologous polynucleotide encoding a suppressor of a cell wall invertase inhibitor are introduced into the nucleus or nuclear genome. In representative embodiments, said heterologous polynucleotide encoding a polypeptide having the enzyme activity of a glycolate dehydrogenase, said heterologous polynucleotide encoding a polypeptide having the enzyme activity of a tartronic semialdehyde reductase, said heterologous polynucleotide encoding a polypeptide having the enzyme activity of a glyoxylate carboligase, and/or said heterologous polynucleotide encoding a polypeptide having the activity of a CO₂ transporter are then localized to the chloroplast. In some embodiments, said heterologous polynucleotide encoding a polypeptide having the activity of a CO₂ transporter can be localized to both the chloroplast and the plasma membrane. In further embodiments, said heterologous polynucleotide encoding a polypeptide having the enzyme activity of a cell wall invertase can be localized to the cell wall. In some representative embodiments, the heterologous polynucleotide encoding a suppressor of a cell wall invertase inhibitor can be introduced into the nucleus or nuclear genome or the cytosol where the suppressor acts to degrade the cell wall invertase inhibitor transcript.

In addition to transformation technology, traditional breeding methods as known in the art (e.g., crossing) can be used to assist in introducing into a single plant each of the heterologous polynucleotides encoding polypeptides having the enzyme activity of glycolate dehydrogenase, and/or tartronic semialdehyde reductase and glyoxylate carboligase, the heterologous polynucleotide encoding a polypeptide having the activity of a CO₂ transporter and/or any other polynucleotides of interest as described herein (e.g., polynucleotides encoding cell wall invertase and a suppressor of an inhibitor of cell wall invertase) to produce a plant, plant part, and/or plant cell comprising and expressing each of said heterologous polynucleotides as described herein.

In some embodiments, the heterologous polynucleotide encoding polypeptides having the enzyme activity of glycolate dehydrogenase, the heterologous polynucleotide encoding a polypeptide having the activity of a CO₂ transporter, the heterologous polynucleotide encoding a polypeptide having the enzyme activity of a tartronic semialdehyde reductase, the heterologous polynucleotide encoding a polypeptide having the enzyme activity of a glyoxylate carboligase, the heterologous polynucleotide encoding a polypeptide having the enzyme activity of a cell wall invertase and/or the heterologous polynucleotide encoding a suppressor of a cell wall invertase inhibitor can be operably linked to a single promoter or to separate and/or different promoters in any combination. Thus, for example, each of the polynucleotides, including each of the three subunits of the glycolate dehydrogenase can be introduced into a plant cell under the control of (operably linked to) separate promoters. In other embodiments, the heterologous polynucleotide encoding a polypeptide having the enzyme activity of a tartronic semialdehyde reductase and the heterologous polynucleotide encoding a polypeptide having the enzyme activity of a glyoxylate carboligase can be operably linked to a single promoter while the heterologous polynucleotide encoding polypeptides having the enzyme activity of glycolate dehydrogenase, the heterologous polynucleotide encoding a polypeptide having the activity of a CO₂ transporter, the heterologous polynucleotide encoding a polypeptide having the enzyme activity of a cell wall invertase and/or the heterologous polynucleotide encoding a suppressor of a cell wall invertase inhibitor can be each operably linked to separate promoters.

In other embodiments, the heterologous polynucleotide encoding polypeptides having the enzyme activity of glycolate dehydrogenase, the heterologous polynucleotide encoding a polypeptide having the enzyme activity of a tartronic semialdehyde reductase and the heterologous polynucleotide encoding a polypeptide having the enzyme activity of a glyoxylate carboligase can be operably linked to a single promoter while the heterologous polynucleotide encoding a polypeptide having the activity of a CO₂ transporter, the heterologous polynucleotide encoding a polypeptide having the enzyme activity of a cell wall invertase and/or the heterologous polynucleotide encoding a suppressor of a cell wall invertase inhibitor can be each operably linked to separate promoters. In some embodiments, the heterologous polynucleotide encoding a polypeptide having the enzyme activity of a cell wall invertase and the heterologous polynucleotide encoding a suppressor of a cell wall invertase inhibitor can be operably linked to a single promoter. In further embodiments, the heterologous polynucleotide encoding polypeptides having the enzyme activity of glycolate dehydrogenase, the heterologous polynucleotide encoding a polypeptide having the enzyme activity of a tartronic semialdehyde reductase and the heterologous polynucleotide encoding a polypeptide having the enzyme activity of a glyoxylate carboligase and the heterologous polynucleotide encoding a polypeptide having the activity of a CO₂ transporter can be operably linked to a single promoter. In still further embodiments, the heterologous polynucleotide encoding a polypeptide having the activity of a CO₂ transporter and the heterologous polynucleotide encoding a polypeptide having the enzyme activity of a cell wall invertase can be operably linked to a single promoter and/or can be overexpressed in said plant. Thus, any combination of promoters with heterologous nucleotides of the invention useful for producing plants having increased seed number, increased assimilate partitioning directed into fruits and/or seeds (and/or other plant part) and/or increased seed size can be utilized.

Any promoter useful for initiation of transcription in a cell of a plant can be used in the expression cassettes of the present invention. A “promoter,” as used herein, is a nucleotide sequence that controls or regulates the transcription of a nucleotide sequence (i.e., a coding sequence) that is operably associated with the promoter. The coding sequence may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. The promoter region may comprise other elements that act as regulators of gene expression. These include a TATA box consensus sequence, and often a CAAT box consensus sequence (Breathnach and Chambon, (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227).

Promoters can include, for example, constitutive, inducible, temporally regulated, developmentally regulated, chemically regulated, tissue-preferred and/or tissue-specific promoters for use in the preparation of recombinant nucleic acid molecules, i.e., “chimeric genes” or “chimeric polynucleotides.” A promoter can be identified in and isolated from the organism to be transformed and then inserted into the nucleic acid construct to be used in transformation of the organism.

The choice of promoter will vary depending on the temporal and spatial requirements for expression, and also depending on the host cell to be transformed. Thus, for example, expression of a heterologous polynucleotide encoding polypeptides having the enzyme activity of glycolate dehydrogenase and a heterologous polynucleotide encoding a polypeptide having the activity of a CO₂ transporter can be in any plant, plant part, (e.g., in leaves, in stalks or stems, in ears, in inflorescences (e.g. spikes, panicles, cobs, etc.), in roots, seeds and/or seedlings, and the like), or plant cells (including algae cells). For example, in the case of a multicellular organism such as a plant where expression in a specific tissue or organ is desired, a tissue-specific or tissue preferred promoter can be used (e.g., a root specific/preferred promoter). In contrast, where expression in response to a stimulus is desired a promoter inducible by stimuli or chemicals can be used. Where continuous expression at a relatively constant level is desired throughout the cells or tissues of an organism a constitutive promoter can be chosen.

Thus, promoters useful with the invention include, but are not limited to, those that drive expression of a nucleotide sequence constitutively, those that drive expression when induced, and those that drive expression in a tissue- or developmentally-specific manner. These various types of promoters are known in the art. Promoters can be identified in and isolated from the plant to be transformed and then inserted into the expression cassette to be used in transformation of the plant.

Non-limiting examples of a promoter include the promoter of the RubisCo small subunit gene 1 (PrbcS1), the promoter of the actin gene (Pactin), the promoter of the nitrate reductase gene (Pnr) and the promoter of duplicated carbonic anhydrase gene 1 (Pdca1) (See, Walker et al. Plant Cell Rep. 23:727-735 (2005); Li et al. Gene 403:132-142 (2007); Li et al. Mol Biol. Rep. 37:1143-1154 (2010)). PrbcS1 and Pactin are constitutive promoters and Pnr and Pdca1 are inducible promoters. Pnr is induced by nitrate and repressed by ammonium (Li et al. Gene 403:132-142 (2007)) and Pdca1 is induced by salt (Li et al. Mol Biol. Rep. 37:1143-1154 (2010)).

Examples of constitutive promoters useful for plants include, but are not limited to, cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter. The constitutive promoter derived from ubiquitin accumulates in many cell types. Ubiquitin promoters have been cloned from several plant species for use in transgenic plants, for example, sunflower (Binet et al., 1991. Plant Science 79: 87-94), maize (Christensen et al., 1989. Plant Molec. Biol. 12: 619-632), and arabidopsis (Norris et al. 1993. Plant Molec. Biol. 21:895-906). The maize ubiquitin promoter (UbiP) has been developed in transgenic monocot systems and its sequence and vectors constructed for monocot transformation are disclosed in the patent publication EP 0 342 926. The ubiquitin promoter is suitable for the expression of the nucleotide sequences of the invention in transgenic plants, especially monocotyledons. Further, the promoter expression cassettes described by McElroy et al. (Mol. Gen. Genet. 231: 150-160 (1991)) can be easily modified for the expression of the nucleotide sequences of the invention and are particularly suitable for use in monocotyledonous hosts.

In some embodiments, tissue specific/tissue preferred promoters can be used for expression of a heterologous polynucleotide in a plant cell. Tissue specific or preferred expression patterns include, but are not limited to, green tissue specific or preferred, root specific or preferred, stem specific or preferred, and flower specific or preferred. Promoters suitable for expression in green tissue include many that regulate genes involved in photosynthesis and many of these have been cloned from both monocotyledons and dicotyledons. In one embodiment, a promoter useful with the invention is the maize PEPC promoter from the phosphoenol carboxylase gene (Hudspeth & Grula, Plant Molec. Biol. 12:579-589 (1989)). Non-limiting examples of tissue-specific promoters include those associated with genes encoding the seed storage proteins (such as β-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP Patent No. 255378). Tissue-specific or tissue-preferential promoters useful for the expression of the nucleotide sequences of the invention in plants, particularly maize, include but are not limited to those that direct expression in root, pith, leaf or pollen. Such promoters are disclosed, for example, in WO 93/07278, herein incorporated by reference in its entirety. Other non-limiting examples of tissue specific or tissue preferred promoters useful with the invention the cotton rubisco promoter disclosed in U.S. Pat. No. 6,040,504; the rice sucrose synthase promoter disclosed in U.S. Pat. No. 5,604,121; the root specific promoter described by de Framond (FEBS 290:103-106 (1991); EP 0 452 269 to Ciba-Geigy); the stem specific promoter described in U.S. Pat. No. 5,625,136 (to Ciba-Geigy) and which drives expression of the maize trpA gene; and the cestrum yellow leaf curling virus promoter disclosed in WO 01/73087.

Additional examples of plant tissue-specific/tissue preferred promoters include, but are not limited to, the root hair-specific cis-elements (RHEs) (Kim et al. The Plant Cell 18:2958-2970 (2006)), the root-specific promoters RCc3 (Jeong et al. Plant Physiol. 153:185-197 (2010)) and RB7 (U.S. Pat. No. 5,459,252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology, 37(8):1108-1115), corn light harvesting complex promoter (Bansal et al. (1992) Proc. Natl. Acad. Sci. USA 89:3654-3658), corn heat shock protein promoter (O'Dell et al. (1985) EMBO J 5:451-458; and Rochester et al. (1986) EMBO J 5:451-458), pea small subunit RuBP carboxylase promoter (Cashmore, “Nuclear genes encoding the small subunit of ribulose-1,5-bisphosphate carboxylase” pp. 29-39 In: Genetic Engineering of Plants (Hollaender ed., Plenum Press 1983; and Poulsen et al. (1986) Mol. Gen. Genet. 205:193-200), Ti plasmid mannopine synthase promoter (Langridge et al. (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612).

Particularly useful for seed-specific expression is the pea vicilin promoter (Czako et al. (1992) Mol. Gen. Genet. 235:33-40; as well as the seed-specific promoters disclosed in U.S. Pat. No. 5,625,136. Useful promoters for expression in mature leaves are those that are switched at the onset of senescence, such as the SAG promoter from Arabidopsis (Gan et al. (1995) Science 270:1986-1988).

In addition, promoters functional in chloroplasts can be used. Non-limiting examples of such promoters include the bacteriophage T3 gene 9 5′ UTR and other promoters disclosed in U.S. Pat. No. 7,579,516. Other promoters useful with the invention include but are not limited to the S-E9 small subunit RuBP carboxylase promoter and the Kunitz trypsin inhibitor gene promoter (Kti3).

In some embodiments of the invention, inducible promoters can be used. Thus, for example, chemical-regulated promoters can be used to modulate the expression of a gene in an organism through the application of an exogenous chemical regulator. Regulation of the expression of nucleotide sequences of the invention via promoters that are chemically regulated enables the polypeptides of the invention to be synthesized only when, for example, a crop of plants are treated with the inducing chemicals. Depending upon the objective, the promoter may be a chemical-inducible promoter, where application of a chemical induces gene expression, or a chemical-repressible promoter, where application of the chemical represses gene expression.

Chemical inducible promoters useful with plants are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid (e.g., the PR1a system), steroid-responsive promoters (see, e.g., the glucocorticoid-inducible promoter in Schena et al. (1991) Proc. Natl. Acad. Sci. USA 88, 10421-10425 and McNellis et al. (1998) Plant J. 14, 247-257) and tetracycline-inducible and tetracycline-repressible promoters (see, e.g., Gatz et al. (1991) Mol. Gen. Genet. 227, 229-237, and U.S. Pat. Nos. 5,814,618 and 5,789,156, Lac repressor system promoters, copper-inducible system promoters, salicylate-inducible system promoters (e.g., the PR1a system), glucocorticoid-inducible promoters (Aoyama et al. (1997) Plant J. 11:605-612), and ecdysone-inducible system promoters.

Other non-limiting examples of inducible promoters include ABA- and turgor-inducible promoters, the auxin-binding protein gene promoter (Schwob et al. (1993) Plant J. 4:423-432), the UDP glucose flavonoid glycosyl-transferase promoter (Ralston et al. (1988) Genetics 119:185-197), the MPI proteinase inhibitor promoter (Cordero et al. (1994) Plant J. 6:141-150), and the glyceraldehyde-3-phosphate dehydrogenase promoter (Kohler et al. (1995) Plant Mol. Biol. 29:1293-1298; Martinez et al. (1989) J. Mol. Biol. 208:551-565; and Quigley et al. (1989) J. Mol. Evol. 29:412-421). Also included are the benzene sulphonamide-inducible (U.S. Pat. No. 5,364,780) and alcohol-inducible (Intl Patent Application Publication Nos. WO 97/06269 and WO 97/06268) systems and glutathione S-transferase promoters. Likewise, one can use any of the inducible promoters described in Gatz (1996) Current Opinion Biotechnol. 7:168-172 and Gatz (1997) Annu. Rev. Plant Physiol. Plant Mol. Biol. 48:89-108. Other chemically inducible promoters useful for directing the expression of the nucleotide sequences of this invention in plants are disclosed in U.S. Pat. No. 5,614,395 herein incorporated by reference in its entirety. Chemical induction of gene expression is also detailed in the published application EP 0 332 104 (to Ciba-Geigy) and U.S. Pat. No. 5,614,395. In some embodiments, a promoter for chemical induction can be the tobacco PR-1a promoter.

In some particular embodiments, promoters useful with algae include, but are not limited to, the promoter of the RubisCo small subunit gene 1 (PrbcS1), the promoter of the actin gene (Pactin), the Arabidopsis Actin2 promoter (Yong-Qiang An et al. 1996 The Plant Journal 10(1):107-121; Yong-Qiang An et al. 2010 Plant Mol Bio Rep 28:481-490) and the tobacco EntCUP4 promoter (Malik et al. 2002 Theor Appl Genet 105:505-514), the promoter of the nitrate reductase gene (Pnr) and the promoter of duplicated carbonic anhydrase gene 1 (Pdca1) (See, Walker et al. Plant Cell Rep. 23:727-735 (2005); Li et al. Gene 403:132-142 (2007); Li et al. Mol Biol. Rep. 37:1143-1154 (2010)), the promoter of the σ⁷⁰-type plastid rRNA gene (Prrn), the promoter of the psbA gene (encoding the photosystem-II reaction center protein D1) (PpsbA), the promoter of the psbD gene (encoding the photosystem-II reaction center protein D2) (PpsbD), the promoter of the psaA gene (encoding an apoprotein of photosystem I) (PpsaA), the promoter of the ATPase alpha subunit gene (PatpA), and promoter of the RuBisCo large subunit gene (PrbcL), and any combination thereof (See, e.g., De Cosa et al. Nat. Biotechnol. 19:71-74 (2001); Daniell et al. BMC Biotechnol. 9:33 (2009); Muto et al. BMC Biotechnol. 9:26 (2009); Surzycki et al. Biologicals 37:133-138 (2009)).

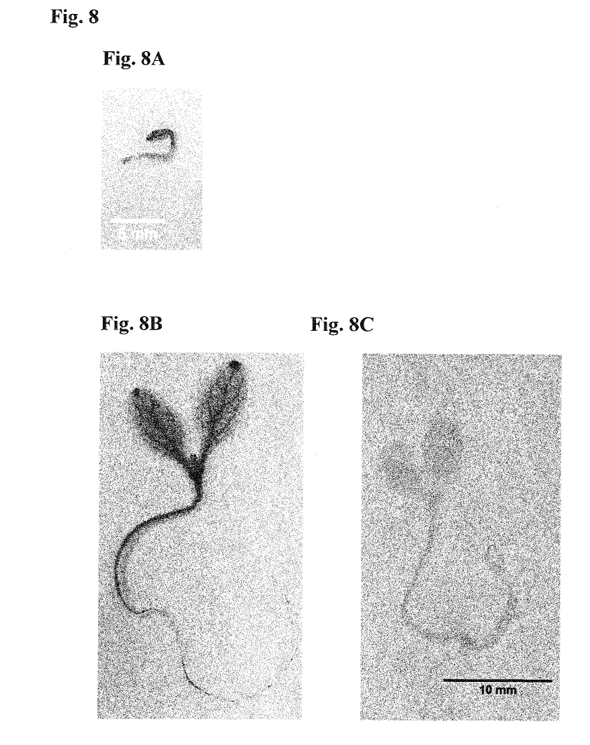

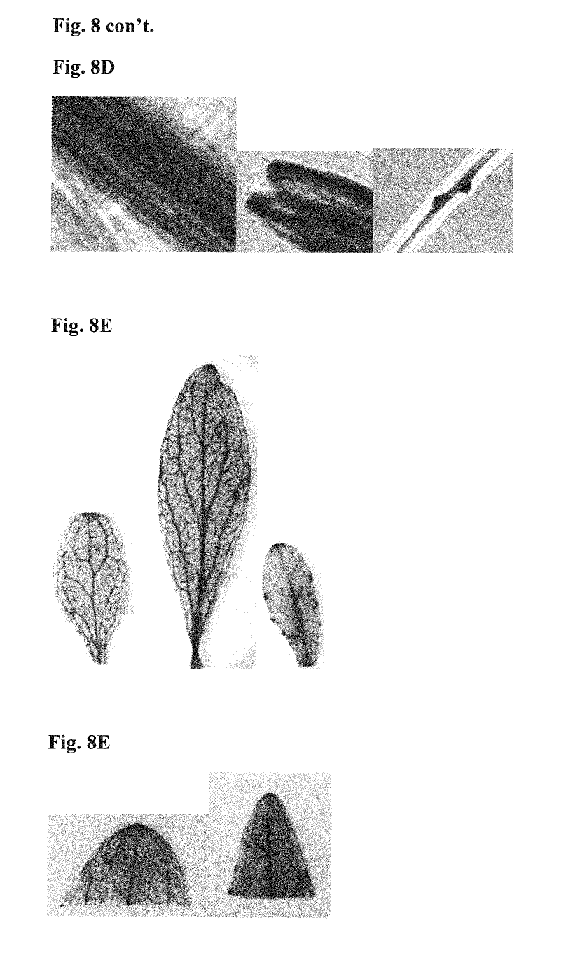

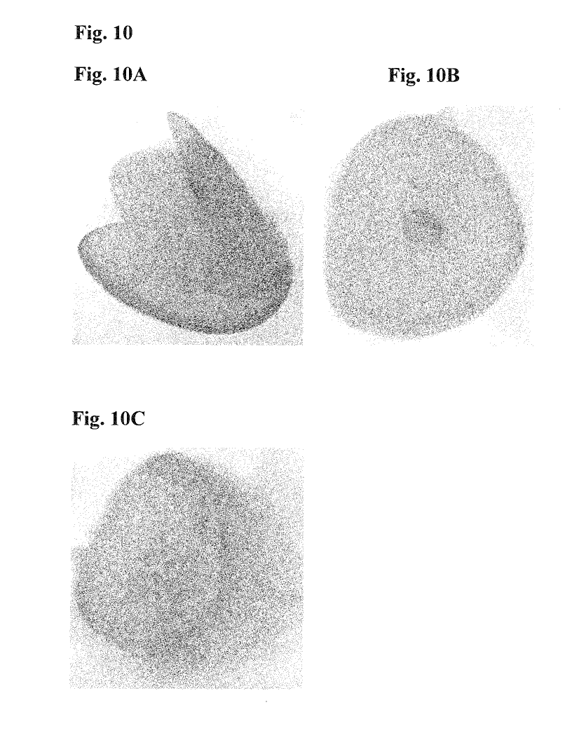

In some embodiments, a promoter useful with the invention can be one or more endogenous promoters of Camelina sativa cell wall invertase inhibitor, Pcwii1 and/or Pcwii2. Pcwii1 (SEQ ID NO:27) can provide tissue specific/tissue preferred expression in the vasculature of various plant tissues (See, FIG. 8). Pcwii2 (SEQ ID NO:28) can provide tissue specific/tissue preferred expression in the root tip and stele (See, FIG. 10). Thus, these two promoters can be useful for providing tissue specific/tissue preferred expression in plants generally. In some embodiments, the present invention provides a heterologous polynucleotide comprising, consisting essentially of, or consisting of a nucleotide sequence of SEQ ID NO:27. In other embodiments, the present invention provides a heterologous polynucleotide comprising, consisting essentially of, or consisting of a nucleotide sequence of SEQ ID NO:28.

In other embodiments, the present invention provides a heterologous polynucleotide comprising, consisting essentially of or consisting of a nucleotide sequence of (a) the nucleotide sequence of SEQ ID NO:27; (b) a nucleotide sequence that is at least about 80% identical to the nucleotide sequence of (a); (c) a nucleotide sequence that differs from the nucleotide sequence of (a) or (b) due to the degeneracy of the genetic code; or (d) any combination of (a), (b) or (c). In still other embodiments, the present invention provides a heterologous polynucleotide comprising, consisting essentially of, or consisting of a nucleotide sequence of (a) the nucleotide sequence of SEQ ID NO:28; (b) a nucleotide sequence that is at least about 80% identical (e.g., at least about 81%, 82%, 83%, 84%, 85%, 86%, 87%, 88%, 89%, 90%, 91%, 92%, 93%, 94%, 95%, 96%, 97%, 98%, 99%, or 100%) to the nucleotide sequence of (a); (c) a nucleotide sequence that differs from the nucleotide sequence of (a) or (b) due to the degeneracy of the genetic code; or (d) any combination of (a), (b) or (c). In some embodiments, the heterologous polynucleotide can be operably linked to a polynucleotide of interest. In additional embodiments, said polynucleotide of interest can be any polynucleotide for which tissue specific/tissue preferred expression (e.g., when expression in the vasculature of plant tissues and/or in the embryo root tip and/or stele is desired). In still other embodiments, the heterologous polynucleotide can be operably linked to one or more polynucleotide sequences encoding additional regulatory elements (e.g., introns, translation enhancers, terminators, Kozak sequences, and the like). In further embodiments, the heterologous nucleotide sequences can be comprised in an expression cassette.

Thus, in additional embodiments, the present invention provides an expression cassette comprising, consisting essentially of, or consisting of: (a) the nucleotide sequence of SEQ ID NO:27; (b) a nucleotide sequence that is at least about 80% identical to the nucleotide sequence of (a); (c) a nucleotide sequence that differs from the nucleotide sequence of (a) or (b) due to the degeneracy of the genetic code; or (d) any combination of (a), (b) or (c); and/or a nucleotide sequence of (a) the nucleotide sequence of SEQ ID NO:28; (b) a nucleotide sequence that is at least about 80% identical to the nucleotide sequence of (a); (c) a nucleotide sequence that differs from the nucleotide sequence of (a) or (b) due to the degeneracy of the genetic code; or (d) any combination of (a), (b) or (c), wherein the heterologous polynucleotide can be operably linked to a polynucleotide of interest and/or to one or more polynucleotide sequences encoding additional regulatory elements as described herein.

In further embodiments, the present invention provides a method of producing a plant having tissue preferential expression of a polynucleotide of interest, comprising introducing into a plant cell a heterologous polynucleotide comprising a nucleotide sequence of: (a) the nucleotide sequence of SEQ ID NO:27 and/or SEQ ID NO:28; (b) a nucleotide sequence that is at least about 80% identical to the nucleotide sequence of (a); (c) a nucleotide sequence that differs from the nucleotide sequence of (a) or (b) due to the degeneracy of the genetic code; or (d) any combination of (a), (b) or (c), wherein the heterologous polynucleotide is operably linked to a polynucleotide of interest; and regenerating the stably transformed plant cell into a stably transformed plant, thereby producing a plant having preferential expression of the polynucleotide of interest in the vasculature of said plant, and/or in the embryo root tip and/or stele of said plant.

In further embodiments, the present invention provides a method of producing a plant having tissue preferential expression of a polynucleotide of interest, comprising introducing into a plant cell an expression cassette comprising a heterologous polynucleotide comprising a nucleotide sequence of: (a) the nucleotide sequence of SEQ ID NO:27 and/or SEQ ID NO:28; (b) a nucleotide sequence that is at least about 80% identical to the nucleotide sequence of (a); (c) a nucleotide sequence that differs from the nucleotide sequence of (a) or (b) due to the degeneracy of the genetic code; or (d) any combination of (a), (b) or (c), wherein the heterologous polynucleotide is operably linked to a polynucleotide of interest, to produce a stably transformed plant cell; and regenerating the stably transformed plant cell into a stably transformed plant, thereby producing a plant having preferential expression of the polynucleotide of interest in the vasculature of said plant, and/or in the embryo root tip and/or stele of said plant.

In some embodiments of the invention, the heterologous polynucleotides of the invention (e.g., polynucleotides encoding polypeptides having the enzyme activity of GDH and/or the activity of a CO₂ transporter, and/or polynucleotides encoding suppressors of inhibitors of cell wall invertase, and the like) can be transformed into the nucleus or into, for example, the chloroplast, using standard techniques known in the art of plant transformation.

Thus, in some embodiments, a heterologous polynucleotide encoding polypeptides having the enzyme activity of glycolate dehydrogenase, a heterologous polynucleotide encoding a polypeptide having the enzyme activity of tartronic semialdehyde reductase, a heterologous polynucleotide encoding a polypeptide having the enzyme activity of glyoxylate carboligase, a heterologous polynucleotide encoding a polypeptide having the activity of a CO₂ transporter, a heterologous polynucleotide encoding a polypeptide having the enzyme activity of a cell wall invertase, and/or a heterologous polynucleotide encoding a suppressor of cwII (e.g., a functional RNA such as RNAi) can be transformed into and expressed in the nucleus and the polypeptides and/or suppressor produced remain in the cytosol. In other embodiments, a heterologous polynucleotide encoding polypeptides having the enzyme activity of glycolate dehydrogenase, a heterologous polynucleotide encoding a polypeptide having the enzyme activity of tartronic semialdehyde reductase, a heterologous polynucleotide encoding a polypeptide having the enzyme activity of glyoxylate carboligase, a heterologous polynucleotide encoding a polypeptide having the activity of a CO₂ transporter, a heterologous polynucleotide encoding a polypeptide having the enzyme activity of a cell wall invertase can be transformed into and expressed in the nucleus and the polypeptides can be targeted to another organelle.

Thus, in particular embodiments, the polypeptide having the enzyme activity of glycolate dehydrogenase is a fusion polypeptide comprising an amino acid sequence that targets said polypeptide to the chloroplast. In further embodiments, the polypeptide having the activity of a CO₂ transporter is a fusion polypeptide comprising an amino acid sequence that targets said polypeptide to the chloroplast and/or the membrane. In still further embodiments, the polypeptide having the enzyme activity of tartronic semialdehyde reductase and the polypeptide having the enzyme activity of glyoxylate carboligase can also be fusion polypeptides comprising an amino acid sequence that targets said polypeptides to the chloroplast.

In representative embodiments, a heterologous polynucleotide encoding the polypeptide having the enzyme activity of glycolate dehydrogenase, a heterologous polynucleotide encoding the polypeptide having the enzyme activity of tartronic semialdehyde reductase, a polypeptide having the enzyme activity of glyoxylate carboligase, and/or a polypeptide having the activity of a CO₂ transporter can be transformed into and expressed in the chloroplast.

A nucleotide sequence encoding a signal peptide may be operably linked at the 5′- or 3′-terminus of a heterologous nucleotide sequence or nucleic acid molecule. Signal peptides (and the targeting nucleotide sequences encoding them) are well known in the art and can be found in public databases such as the “Signal Peptide Website: An Information Platform for Signal Sequences and Signal Peptides.” (www.signalpeptide.de); the “Signal Peptide Database” (proline.bic.nus.edu.sg/spdb/index.html) (Choo et al., BMC Bioinformatics 6:249 (2005)(available on www.biomedcentral.com/1471-2105/6/249/abstract); ChloroP (www.cbs.dtu.dk/services/ChloroP/; predicts the presence of chloroplast transit peptides (cTP) in protein sequences and the location of potential cTP cleavage sites); LipoP (www.cbs.dtu.dk/services/LipoP/; predicts lipoproteins and signal peptides in Gram negative bacteria); MITOPROT (ihg2 helmholtz-muenchen.de/ihg/mitoprot.html; predicts mitochondrial targeting sequences); PlasMit (gecco.org.chemie.uni-frankfurt.de/plasmit/index.html; predicts mitochondrial transit peptides in Plasmodium falciparum); Predotar (urgi.versailles.inra.fr/predotar/predotar.html; predicts mitochondrial and plastid targeting sequences); PTS1 (mendel.imp.ac.at/mendeljsp/sat/pts1/PTS1predictor.jsp; predicts peroxisomal targeting signal 1 containing proteins); SignalP (www.cbs.dtu.dk/services/SignalP/; predicts the presence and location of signal peptide cleavage sites in amino acid sequences from different organisms: Gram-positive prokaryotes, Gram-negative prokaryotes, and eukaryotes). The SignalP method incorporates a prediction of cleavage sites and a signal peptide/non-signal peptide prediction based on a combination of several artificial neural networks and hidden Markov models; and TargetP (www.cbs.dtu.dk/services/TargetP/); predicts the subcellular location of eukaryotic proteins—the location assignment is based on the predicted presence of any of the N-terminal presequences: chloroplast transit peptide (cTP), mitochondrial targeting peptide (mTP) or secretory pathway signal peptide (SP)). (See also, von Heijne, G., Eur J Biochem 133 (1) 17-21 (1983); Martoglio et al. Trends Cell Biol 8 (10):410-5 (1998); Hegde et al. Trends Biochem Sci 31(10):563-71 (2006); Dultz et al. J. Biol Chem 283(15):9966-76 (2008); Emanuelsson et al. Nature Protocols 2(4) 953-971(2007); Zuegge et al. 280(1-2):19-26 (2001); Neuberger et al. J Mol Biol. 328(3):567-79 (2003); and Neuberger et al. J Mol Biol. 328(3):581-92 (2003)).

Exemplary signal peptides include, but are not limited to those provided in Table 1.

TABLE 1
Amino acid sequences of representative signal peptides.

| Source | Sequence | Target |
|---|---|---|
| Rubisco small | MASSVLSSAAVATRSNVAQANMVAPFTGLKSAASF | chloroplast |
| subunit (tobacco) | PVSRKQNLDITSIASNGGRVQC (SEQ ID NO: 29) | |
| Saccharomyces | MLSLRQSTRFFKPATRTLCSSRYLL (SEQ ID NO: 30) | mitochondria |
| cerevisiae cox4 | | |
| Arabidopsis | MYLTASSSASSSIIRAASSRSSSLFSFRSVLSPSVSSTSP | mitochondria |
| aconitase | SSLLARRSFGTISPAFRRWSHSFHSKPSPFRFTSQIRA | |
| | (SEQ ID NO: 31) | |
| Yeast aconitase | MLSARSAIKRPIVRGLATV (SEQ ID NO: 32) | mitochondria |
| Arabidopsis | MRILPKSGGGALCLLFVFALCSVAHS | cell |
| proline-rich | (SEQ ID NO: 33) | wall/secretory |
| protein 2 | | pathway |
| (AT2G21140) | | |
| Arabidopsis | MLRTVSCLASRSSSSLFFRFFRQFPRSYMSLTSSTAAL | mitochondria |
| presequence | RVPSRNLRRISSPSVAGRRLLLRRGLRIPSAAVRSVN | and |
| proteasel | GQFSRLSVRA (SEQ ID NO: 34) | chloroplast |
| (AT3G19170) | | |
| Chlamydomonas | MALVARPVLSARVAASRPRVAARKAVRVSAKYGEN | chloroplast |
| reinhardtii-(Stroma- | (SEQ ID NO: 35) | |
| targeting cTPs: | MQALSSRVNIAAKPQRAQRLVVRAEEVKA | |
| photosystem I (PSI) | (SEQ ID NO: 36) | |
| subunits P28, P30, | MQTLASRPSLRASARVAPRRAPRVAVVTKAALDPQ | |
| P35 and P37, | (SEQ ID NO: 37) | |
| respectively) | MQALATRPSAIRPTKAARRSSVVVRADGFIG | |
| | (SEQ ID NO: 38) | |
| C. reinhardtii- | MAFALASRKALQVTCKATGKKTAAKAAAPKSSGVE | chloroplast |
| chlorophyll a/b | FYGPNRAKWLGPYSEN (SEQ ID NO: 39) | |
| protein (cabII-1) | | |
| C. reinhardti- | MAAVIAKSSVSAAVARPARSSVRPMAALKPAVKAA | chloroplast |
| Rubisco small | PVAAPAQANQMMVWT (SEQ ID NO: 40) | |
| subunit | | |
| C. reinhardtii- | MAAMLASKQGAFMGRSSFAPAPKGVASRGSLQVVA | chloroplast |
| ATPase-γ | GLKEV (SEQ ID NO: 41) | |
| Rubisco small | MASSMLSSATMVASPAQATMVAPFNGLKSSAAFPA | chloroplast |
| subunit Arabidopsis | TRKANNDITSITSNGGRVNC (SEQ ID NO: 42) | |
| Biotin carboxyl | MASSSFSVTSPAAAASVYAVTQTS SIEPIQNRSRRVS | chloroplast |
| carrier protein | FRLSAKPKLRFLSKPSRSSYPVVKA (SEQ ID NO: 43) | |
| Arabidopsis | | |
| Arabidopsis thaliana | CVVQ (SEQ ID NO: 44) | membrane |
| abscisic acid | | |
| receptor PYL10 | | |

Thus, in representative embodiments of the invention, the polypeptide having the enzyme activity of glycolate dehydrogenase, the polypeptide having the enzyme activity of tartronic semialdehyde reductase, the polypeptide having the enzyme activity of glyoxylate carboligase, the polypeptide having the activity of a CO₂ transporter, and/the the polypeptide having the activity of a cell wall invertase to be expressed in a plant, plant cell, plant part can be fusion polypeptide comprising an amino acid sequence that targets said polypeptide to the chloroplast (e.g., a chloroplast signal peptide). In some embodiments, said chloroplast signal peptide can be encoded by an amino acid sequence that includes, but is not limited to, the amino acid sequence of SEQ ID NO:29, SEQ ID NO:35, SEQ ID NO:36, SEQ ID NO:37, SEQ ID NO:38, SEQ ID NO:39, SEQ ID NO:40, or SEQ ID NO:41.

In other embodiments of the invention, a heterologous polynucleotide encoding a CO₂ transporter (e.g., aquaporin) to be expressed in a plant, plant part or plant cell can be operably linked to a mitochondrial targeting sequence encoding a mitochondrial signal peptide, optionally wherein said mitochondrial signal peptide is encoded by an amino acid sequence that includes, but is not limited to, the amino acid sequence of SEQ ID NO:30, SEQ ID NO:31, or SEQ ID NO:32.

In further embodiments, a heterologous polynucleotide encoding a polypeptide having the enzyme activity of a cell wall invertase to be expressed in a plant, plant part or plant cell can be operably linked to a cell wall targeting sequence encoding a cell wall signal peptide, optionally wherein said cell wall signal peptide is encoded by an amino acid sequence that includes, but is not limited to, the amino acid sequence of SEQ ID NO:33.

In some embodiments, a polypeptide having the enzyme activity of glycolate dehydrogenase, a polypeptide having the enzyme activity of tartronic semialdehyde reductase, a polypeptide having the enzyme activity of glyoxylate carboligase, a heterologous polynucleotide encoding a CO₂ transporter to be expressed in a plant, plant part or plant cell can be operably linked to a membrane targeting sequence encoding a membrane signal peptide, optionally wherein said membrane signal peptide is encoded by an amino acid sequence that includes, but is not limited to, the amino acid sequence of SEQ ID NO:44. In some embodiments, wherein when the heterologous polynucleotide encoding a CO₂ transporter is targeted to a membrane, the CO₂ transporter can be either linked directly to the membrane or to the membrane via a linkage to a membrane associated protein. In representative embodiments, a membrane associated protein includes but is not limited to the plasma membrane NADH oxidase (RbohA) (for respiratory burst oxidase homolog A) (Keller et al. The Plant Cell Online 10: 255-266 (1998)), annexinl (ANN1) from Arabidopsis thaliana (Laohavisit et al. Plant Cell Online 24: 1522-1533 (2012)), and/or the nitrate transporter CHL1 (AtNRT1.1) (Tsay et al. “The Role of Plasma Membrane Nitrogen Transporters in Nitrogen Acquisition and Utilization,” In, The Plant Plasma Membrane 19:223-236 Springer Berlin/Heidelberg (2011)).

Targeting to a membrane is similar to targeting to an organelle. Thus, specific sequences on a protein (targeting sequences or motifs) can be recognized by a transporter, which then imports the protein into an organelle or in the case of membrane association, the transporter can guide the protein to and associate it with a membrane. Thus, for example, a specific cysteine residue on a C-terminal motif of a protein can be modified post-translation where an enzyme (prenyltransferases) then attaches a hydrophobic molecule (geranylgeranyl or farnesyl) (See, e.g., Running et al. Proc Natl Acad Sci USA 101: 7815-7820 (2004); Maurer-Stroh et al. Genome Biology 4:212 (2003)). This hydrophobic addition guides and associates the protein to a membrane (in case of the cytosol, the membrane would be the plasma membrane or the cytosolic side of the nuclear membrane (Polychronidou et al. Molecular Biology of the Cell 21: 3409-3420 (2010)). More specifically, in representative embodiments, a protein prenyltransferase can catalyze the covalent attachment of a 15-carbon farnesyl or 20-carbon geranylgeranyl isoprenoid to C-terminal cysteines of selected proteins carrying a CaaX motif where C=cysteine; a=aliphatic amino acid; x=any amino acid. For plants, this motif most often is CVVQ (SEQ ID NO:44). The addition of prenyl groups facilitates membrane association and protein-protein interactions of the prenylated proteins.

In still other embodiments of the invention, a signal peptide can direct a polypeptide of the invention to more than one organelle (e.g., dual targeting). Thus, in some embodiments, a signal peptide that can target a polypeptide of the invention to more than one organelle is encoded by an amino acid sequence that includes, but is not limited to, the amino acid sequence of SEQ ID NO:34.

In addition to promoters operably linked to a heterologous polynucleotide of the invention, an expression cassette also can include other regulatory sequences. As used herein, “regulatory sequences” means nucleotide sequences located upstream (5′ non-coding sequences), within or downstream (3′ non-coding sequences) of a coding sequence, and which influence the transcription, RNA processing or stability, or translation of the associated coding sequence. Regulatory sequences include, but are not limited to, enhancers, introns, translation leader sequences, translation termination sequences, and polyadenylation signal sequences, as described herein.

Thus, in some embodiments of the present invention, the expression cassettes can include at least one intron. An intron useful with this invention can be an intron identified in and isolated from a plant to be transformed and then inserted into the expression cassette to be used in transformation of the plant. As would be understood by those of skill in the art, the introns as used herein comprise the sequences required for self excision and are incorporated into the nucleic acid constructs in frame. An intron can be used either as a spacer to separate multiple protein-coding sequences in one nucleic acid construct, or an intron can be used inside one protein-coding sequence to stabilize the mRNA. If they are used within a protein-coding sequence, they are inserted “in-frame” with the excision sites included.

Non-limiting examples of introns useful with the present invention can be introns from the RuBisCO small subunit (rbcS) gene, the RuBisCO large subunit (rbcL) gene, the actin gene, the nitrate reductase gene (nr), the duplicated carbonic anhydrase gene 1 (Tdca1), the psbA gene, the atpA gene, or any combination thereof.

In some embodiments of the invention, an expression cassette can comprise an enhancer sequence. Enhancer sequences can be derived from, for example, any intron from any highly expressed gene. In particular embodiments, an enhancer sequence usable with this invention includes, but is not limited to, the nucleotide sequence of ggagg (e.g., ribosome binding site).

An expression cassette also can optionally include a transcriptional and/or translational termination region (i.e., termination region) that is functional in plants, yeast or bacteria. A variety of transcriptional terminators are available for use in expression cassettes and are responsible for the termination of transcription beyond the heterologous polynucleotide of interest and correct mRNA polyadenylation. The termination region may be native to the transcriptional initiation region, may be native to the operably linked nucleotide sequence of interest, may be native to the host cell, or may be derived from another source (i.e., foreign or heterologous to the promoter, the nucleotide sequence of interest, the host cell, or any combination thereof). Non-limiting examples of transcriptional terminators useful for plants can be a CAMV 35S terminator, a tm1 terminator, a nopaline synthase terminator and/or a pea rbcs E9 terminator, a RubisCo small subunit gene 1 (TrbcS1) terminator, an actin gene (Tactin) terminator, a nitrate reductase gene (Tnr) terminator, and/or aa duplicated carbonic anhydrase gene 1 (Tdca1) terminator.

Further non-limiting examples of terminators useful with this invention for expression of the heterologous polynucleotides of the invention or other heterologous polynucleotides of interest in algae include a terminator of the psbA gene (TpsbA), a terminator of the psaA gene (encoding an apoprotein of photosystem I) (TpsaA), a terminator of the psbD gene (TpsbD), a RuBisCo large subunit terminator (TrbcL), a terminator of the σ⁷⁰-type plastid rRNA gene (Trrn), and/or a terminator of the ATPase alpha subunit gene (TatpA).

An expression cassette of the invention also can include a nucleotide sequence for a selectable marker, which can be used to select a transformed plant, plant part and/or plant cell. As used herein, “selectable marker” means a nucleotide sequence that when expressed imparts a distinct phenotype to a plant, plant part and/or plant cell expressing the marker and thus allows such a transformed plant, plant part, and/or plant cell to be distinguished from that which does not have the marker. Such a nucleotide sequence may encode either a selectable or screenable marker, depending on whether the marker confers a trait that can be selected for by chemical means, such as by using a selective agent (e.g., an antibiotic, herbicide, or the like), or whether the marker is simply a trait that one can identify through observation or testing, such as by screening (e.g., the R-locus trait). Of course, many examples of suitable selectable markers are known in the art and can be used in the expression cassettes described herein.

Examples of selectable markers include, but are not limited to, a nucleotide sequence encoding aadA (i.e., spectinomycin and streptomycin resistance), a nucleotide sequence encoding neo (i.e., kanamycin resistance), a nucleotide sequence encoding aphA6 (i.e., kanamycin resistance), a nucleotide sequence encoding nptII (i.e., kanamycin resistance), a nucleotide sequence encoding bar (i.e., phosphinothricin resistance), a nucleotide sequence encoding cat (i.e., chloramphenicol resistance), a nucleotide sequence encoding badh (i.e., betaine aldehyde resistance), a nucleotide sequence encoding egfp, (i.e., enhanced green fluorescence protein), a nucleotide sequence encoding gfp (i.e., green fluorescent protein), a nucleotide sequence encoding luc (i.e., luciferase), a nucleotide sequence encoding ble (bleomycin resistance), a nucleotide sequence encoding ereA (erythromycin resistance), and any combination thereof.

Further examples of selectable markers useful with the invention include, but are not limited to, a nucleotide sequence encoding an altered 5-enolpyruvylshikimate-3-phosphate (EPSP) synthase, which confers resistance to glyphosate (Hinchee et al. (1988) Biotech. 6:915-922); a nucleotide sequence encoding a nitrilase such as bxn from Klebsiella ozaenae that confers resistance to bromoxynil (Stalker et al. (1988) Science 242:419-423); a nucleotide sequence encoding an altered acetolactate synthase (ALS) that confers resistance to imidazolinone, sulfonylurea or other ALS-inhibiting chemicals (EP Patent Application No. 154204); a nucleotide sequence encoding a methotrexate-resistant dihydrofolate reductase (DHFR) (Thillet et al. (1988) J Biol. Chem. 263:12500-12508); a nucleotide sequence encoding a dalapon dehalogenase that confers resistance to dalapon; a nucleotide sequence encoding a mannose-6-phosphate isomerase (also referred to as phosphomannose isomerase (PMI)) that confers an ability to metabolize mannose (U.S. Pat. Nos. 5,767,378 and 5,994,629); a nucleotide sequence encoding an altered anthranilate synthase that confers resistance to 5-methyl tryptophan; and/or a nucleotide sequence encoding hph that confers resistance to hygromycin.

Additional selectable markers include, but are not limited to, a nucleotide sequence encoding β-glucuronidase or uidA (GUS) that encodes an enzyme for which various chromogenic substrates are known; an R-locus nucleotide sequence that encodes a product that regulates the production of anthocyanin pigments (red color) in plant tissues (Dellaporta et al., “Molecular cloning of the maize R-nj allele by transposon-tagging with Ac” 263-282 In: Chromosome Structure and Function: Impact of New Concepts, 18th Stadler Genetics Symposium (Gustafson & Appels eds., Plenum Press 1988)); a nucleotide sequence encoding β-lactamase, an enzyme for which various chromogenic substrates are known (e.g., PADAC, a chromogenic cephalosporin) (Sutcliffe (1978) Proc. Natl. Acad. Sci. USA 75:3737-3741); a nucleotide sequence encoding xylE that encodes a catechol dioxygenase (Zukowsky et al. (1983) Proc. Natl. Acad. Sci. USA 80:1101-1105); a nucleotide sequence encoding tyrosinase, an enzyme capable of oxidizing tyrosine to DOPA and dopaquinone, which in turn condenses to form melanin (Katz et al. (1983) J Gen. Microbiol. 129:2703-2714); a nucleotide sequence encoding β-galactosidase, an enzyme for which there are chromogenic substrates; a nucleotide sequence encoding luciferase (lux) that allows for bioluminescence detection (Ow et al. (1986) Science 234:856-859); a nucleotide sequence encoding Bla that confers ampicillin resistance; or a nucleotide sequence encoding aequorin which may be employed in calcium-sensitive bioluminescence detection (Prasher et al. (1985) Biochem. Biophys. Res. Comm. 126:1259-1268), and/or any combination thereof. One of skill in the art is capable of choosing a suitable selectable marker for use in an expression cassette of this invention.

An expression cassette comprising a heterologous polynucleotide of the invention (e.g., polynucleotide(s) encoding polypeptides encoding glycolate dehydrogenase, a CO₂ transporter and/or a polynucleotide encoding a suppressor of cwII), also can optionally include polynucleotides that encode other desired traits. Such desired traits can be polynucleotides which confer high light tolerance, increased drought tolerance, increased flooding tolerance, increased tolerance to soil contaminants, increased yield, modified fatty acid composition of the lipids, increased oil production in seed, increased and modified starch production in seeds, increased and modified protein production in seeds, modified tolerance to herbicides and pesticides, production of terpenes, increased seed number, and/or other desirable traits for agriculture or biotechnology.

Such polynucleotides can be stacked with any combination of nucleotide sequences to create plants, plant parts and/or plant cells having the desired phenotype. Stacked combinations can be created by any method including, but not limited to, any conventional methodology (e.g., cross breeding for plants), or by genetic transformation. If stacked by genetic transformation, nucleotide sequences encoding additional desired traits can be combined at any time and in any order. For example, a transgenic plant comprising one or more desired traits can be used as the target to introduce further traits by subsequent transformation. The additional nucleotide sequences can be introduced simultaneously in a co-transformation protocol with a nucleotide sequence, nucleic acid molecule, nucleic acid construct, and/or other composition of the invention, provided by any combination of expression cassettes. For example, if two nucleotide sequences will be introduced, they can be incorporated in separate cassettes (trans) or can be incorporated on the same cassette (cis). Expression of the nucleotide sequences can be driven by a single promoter or by separate promoters, which can be the same or different, or a combination thereof. It is further recognized that nucleotide sequences can be stacked at a desired genomic location using a site-specific recombination system. See, e.g., Int'l Patent Application Publication Nos. WO 99/25821; WO 99/25854; WO 99/25840; WO 99/25855 and WO 99/25853.

By “operably linked” or “operably associated,” it is meant that the indicated elements are functionally related to each other, and are also generally physically related. Thus, the term “operably linked” or “operably associated” as used herein, refers to nucleotide sequences on a single nucleic acid molecule that are functionally associated. Therefore, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, sequences can be present between a promoter and a nucleotide sequence, and the promoter can still be considered “operably linked” to the nucleotide sequence.

Any plant (or groupings of plants, for example, into a genus or higher order classification) can be employed in practicing this invention including an angiosperm, a gymnosperm, a monocot, a dicot, a C3, C4, CAM plant, a microalgae, and/or a macroalgae.

The term “plant part,” as used herein, includes but is not limited to reproductive tissues (e.g., petals, sepals, stamens, pistils, receptacles, anthers, pollen, flowers, fruits, flower bud, ovules, seeds, embryos, nuts, kernels, ears, cobs and husks); vegetative tissues (e.g., petioles, stems, roots, root hairs, root tips, pith, coleoptiles, stalks, shoots, branches, bark, apical meristem, axillary bud, cotyledon, hypocotyls, and leaves); vascular tissues (e.g., phloem and xylem); specialized cells such as epidermal cells, parenchyma cells, chollenchyma cells, schlerenchyma cells, stomates, guard cells, cuticle, mesophyll cells; callus tissue; and cuttings. The term “plant part” also includes plant cells, including plant cells that are intact in plants and/or parts of plants, plant protoplasts, plant tissues, plant organs, plant cell tissue cultures, plant calli, plant clumps, and the like. As used herein, “shoot” refers to the above ground parts including the leaves and stems. As used herein, the term “tissue culture” encompasses cultures of tissue, cells, protoplasts and callus.

As used herein, “plant cell” refers to a structural and physiological unit of the plant, which typically comprise a cell wall but also includes protoplasts. A plant cell of the present invention can be in the form of an isolated single cell or can be a cultured cell or can be a part of a higher-organized unit such as, for example, a plant tissue (including callus) or a plant organ. In some embodiments, a plant cell can be an algal cell.

In some embodiments of this invention, a plant, plant part or plant cell can be from a genus including, but not limited to, the genus of Camelina, Sorghum, Brassica, Allium, Armoracia, Poa, Agrostis, Lolium, Festuca, Calamogrostis, Deschampsia, Spinacia, Beta, Pisum, Chenopodium, Glycine, Helianthus, Pastinaca, Daucus, Petroselium, Populus, Prunus, Castanea, Eucalyptus, Acer, Quercus, Salix, Juglans, Picea, Pinus, Abies, Lemna, Wolffia, Spirodela, Oryza or Gossypium.

In other embodiments, a plant, plant part or plant cell can be from a species including, but not limited to, the species of Camelina alyssum (Mill.) Thell., Camelina microcarpa Andrz. ex DC., Camelina rumelica Velen., Camelina sativa (L.) Crantz, Sorghum bicolor (e.g., Sorghum bicolor L. Moench), Glycine max, Gossypium hirsutum, Brassica oleracea, Brassica rapa, Brassica napus, Raphanus sativus, Armoracia rusticana, Allium sative, Allium cepa, Populus grandidentata, Populus tremula, Populus tremuloides, Prunus serotina, Prunus pensylvanica, Castanea dentate, Populus balsamifer, Populus deltoids, Acer Saccharum, Acer nigrum, Acer negundo, Acer rubrum, Acer saccharinum, Acer pseudoplatanus or Oryza sativa. In additional embodiments, the plant, plant part or plant cell can be, but is not limited to, a plant of, or a plant part, or plant cell from wheat, barley, oats, turfgrass (bluegrass, bentgrass, ryegrass, fescue), feather reed grass, tufted hair grass, spinach, chard, quinoa, lettuce, sunflower (Helianthus annuus), peas (Pisum sativum), parsnips (Pastinaca sativa), carrots (Daucus carota), parsley (Petroselinum crispum), duckweed, pine, spruce, fir, eucalyptus, oak, walnut, or willow. In particular embodiments, the plant, plant part and/or plant cell can be from Camelina sativa.

Additional non-limiting examples of plants can include vegetable crops, including artichokes, kohlrabi, arugula, leeks, asparagus, lettuce (e.g., head, leaf, romaine), bok choy, malanga, melons (e.g., muskmelon, watermelon, crenshaw, honeydew, cantaloupe), cole crops (e.g., brussels sprouts, cabbage, cauliflower, broccoli, collards, kale, chinese cabbage, bok choy) cardoni, carrots, napa, okra, onions, celery, parsley, chick peas, parsnips, chicory, peppers, potatoes, cucurbits (e.g., marrow, cucumber, zucchini, squash, pumpkin), radishes, dry bulb onions, rutabaga, eggplant (also called brinjal), salsify, escarole, shallots, endive, garlic, spinach, green onions, squash, greens, beet (sugar beet and fodder beet), sweet potatoes, swiss chard, horseradish, tomatoes, turnips, and spices; a fruit and/or vine crop such as apples, apricots, cassava, cherries, nectarines, peaches, pears, plums, prunes, cherry, quince, fig, almonds, macadamia, chestnuts, filberts, cashews, pecans, pistachios, walnuts, citrus, blueberries, boysenberries, cranberries, currants, loganberries, raspberries, strawberries, blackberries, grapes, avocados, bananas, kiwi, persimmons, pomegranate, avocado, pineapple, tropical fruits, pomes, melon, guava, papaya, and lychee; a field crop plant such as clover, alfalfa, evening primrose, meadow foam, corn/maize (field, sweet, popcorn), hops, jojoba, peanuts, rice, safflower, small grains (barley, oats, rye, wheat, etc.), sorghum, tobacco, kapok, a leguminous plant (beans, lentils, peas, soybeans), an oil plant (rape, mustard, canola, poppy, olive, sunflower, coconut, castor oil plant, cocoa bean, groundnut), Arabidopsis, a fibre plant (cotton, flax, hemp, jute), lauraceae (cinnamon, camphor), or a plant such as coffee, sugar cane, tea, and natural rubber plants.

Additional non-limiting examples of plants useful with this invention include broad-leaved trees and evergreen trees (e.g., conifers), turfgrasses (e.g., for ornamental, recreational or forage purposes (e.g., zoysia grass, bent grass, fescue grass, bluegrass, St. Augustine grass, Bermuda grass, buffalo grass, rye grass, and orchard grass), and biomass grasses (e.g., switchgrass and Miscanthus); ornamental plants (e.g., azalea, hydrangea, hibiscus, rose, tulip, daffodil, petunia, carnation, poinsettia and chrysanthemum, cactus, succulent).

In further embodiments, a plant and/or plant cell can be an algae or algae cell from a class including, but not limited to, the class of Bacillariophyceae (diatoms), Haptophyceae, Phaeophyceae (brown algae), Rhodophyceae (red algae) or Glaucophyceae (red algae). In still other embodiments, a plant and/or plant cell can be an algae or algae cell from a genus including, but not limited to, the genus of Achnanthidium, Actinella, Nitzschia, Nupela, Geissleria, Gomphonema, Planothidium, Halamphora, Psammothidium, Navicula, Eunotia, Stauroneis, Chlamydomonas, Dunaliella, Nannochloris, Nannochloropsis, Scenedesmus, Chlorella, Cyclotella, Amphora, Thalassiosira, Phaeodactylum, Chrysochromulina, Prymnesium, Thalassiosira, Phaeodactylum, Glaucocystis, Cyanophora, Galdieria, or Porphyridium. Additional nonlimiting examples of genera and species of diatoms useful with this invention are provided by the US Geological Survey/Institute of Arctic and Alpine Research at westerndiatoms.colorado.edu/species.

Any nucleotide sequence to be transformed into a plant, plant part and/or plant cell can be modified for codon usage bias using species specific codon usage tables. The codon usage tables are generated based on a sequence analysis of the most highly expressed genes for the species of interest. When the nucleotide sequences are to be expressed in the nucleus, the codon usage tables are generated based on a sequence analysis of highly expressed nuclear genes for the species of interest. The modifications for the nucleotide sequences for selection are determined by comparing the species specific codon usage table with the codons present in the native polynucleotide sequences. In those embodiments in which each of codons in native polynucleotide sequence for selection are sufficiently used, then no modifications are needed (e.g., a frequency of more than 30% for a codon used for a specific amino acid in that species would indicate no need for modification). In other embodiments, wherein up to 3 nucleotides have to be modified in the polynucleotide sequence, site-directed mutagenesis can be used according to methods known in the art (Zheng et al. Nucleic Acids Res. 32:e115 (2004); Dammai, Meth. Mol. Biol 634:111-126 (2010); Davis and Vierstra. Plant Mol. Biol. 36(4): 521-528 (1998)). In still other embodiments, wherein more than three nucleotide changes are necessary, a synthetic nucleotide sequence can be generated using the same codon usage as the highly expressed genes that were used to develop the codon usage table.

The term “transformation” as used herein refers to the introduction of a heterologous polynucleotide into a cell. Transformation of a plant, plant part, plant cell, yeast cell and/or bacterial cell may be stable or transient.

“Transient transformation” in the context of a polynucleotide means that a polynucleotide is introduced into the cell and does not integrate into the genome of the cell.

By “stably introducing” or “stably introduced” in the context of a polynucleotide introduced into a cell it is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein also includes the nuclear and the plastid genome, and therefore includes integration of the nucleic acid into, for example, the chloroplast genome. Stable transformation as used herein can also refer to a transgene that is maintained extrachromasomally, for example, as a minichromosome. The phrase “a stably transformed plant, plant part, and/or plant cell expressing said one or more polynucleotide sequences” and similar phrases used herein, means that the stably transformed plant, plant part, and/or plant cell comprises the one or more polynucleotide sequences and that said one or more polynucleotide sequences are functional in said stably transformed plant, plant part, and/or plant cell.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a Northern blot hybridization assay of RNA of the cell with nucleic acid sequences which specifically hybridize with a nucleotide sequence of a transgene introduced into a plant or other organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols that are well known in the art.

A heterologous polynucleotide encoding polypeptides having the enzyme activity of glycolate dehydrogenase, a heterologous polynucleotide encoding a polypeptide having the enzyme activity of a CO₂ transporter (e.g., aquaporin), a heterologous polynucleotide encoding a polypeptide having the enzyme activity of a tartronic semialdehyde reductase, a heterologous polynucleotide encoding a polypeptide having the enzyme activity of an glyoxylate carboligase, a heterologous polynucleotide encoding a polypeptide having the enzyme activity of a cell wall invertase and/or a heterologous polynucleotide encoding a suppressor of cwII as described herein; and/or functional fragments thereof (e.g., a functional fragment of the nucleotide sequences of SEQ ID NOs:1, 3, 5, 7, 8, 9, 10, 12, 14, 16, 18, 20, 22, 23, 24, 25, 26, 27, 28, 45, 47, 49, 51, 53 and/or any combination thereof, or a functional fragment of the amino acid sequences of SEQ ID NOs:2, 4, 6, 11, 13, 15, 17, 19, 21, 46, 48, 50, 51, 52, 54 and/or any combination thereof) can be introduced into a cell of a plant by any method known to those of skill in the art. In some embodiments of the invention, transformation of a cell comprises nuclear transformation. In other embodiments, transformation of a cell comprises plastid transformation (e.g., chloroplast transformation).

Procedures for transforming plants are well known and routine in the art and are described throughout the literature. Non-limiting examples of transformation methods include transformation via bacterial-mediated nucleic acid delivery (e.g., via Agrobacteria), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)). General guides to the transformation of yeast include Guthrie and Fink (1991)′ (Guide to yeast genetics and molecular biology. In Methods in Enzymology, (Academic Press, San Diego) 194:1-932) and guides to methods related to the transformation of bacteria include Aune and Aachmann (Appl. Microbiol Biotechnol 85:1301-1313 (2010)).

A polynucleotide therefore can be introduced into a plant, plant part, plant cell in any number of ways that are well known in the art. The methods of the invention do not depend on a particular method for introducing one or more nucleotide sequences into a plant, only that they gain access to the interior the cell. Where more than polynucleotide is to be introduced, they can be assembled as part of a single nucleic acid construct, or as separate nucleic acid constructs, and can be located on a single nucleic acid construct or separate nucleic acid constructs. Accordingly, the polynucleotide can be introduced into the cell of interest in a single transformation event, or in separate transformation events, or, alternatively, a polynucleotide can be incorporated into a plant as part of a breeding protocol.

In some embodiments, when a plant part or plant cell is stably transformed, it can then be used to regenerate a stably transformed plant comprising one or more heterologous polynucleotides encoding a polypeptide having the enzyme activity of a glycolate dehydrogenase and/or a heterologous polynucleotide encoding a polypeptide having the activity of a CO₂ transporter (e.g., aquaporin) and/or a heterologous polynucleotide encoding a suppressor of a cell wall invertase inhibitor (cwII) as described herein, and/or other polynucleotides of interest as described herein (e.g, TSR, GCL), and/or any combination thereof in its genome. Means for regeneration can vary from plant species to plant species, but generally a suspension of transformed protoplasts or a petri plate containing transformed explants is first provided. Callus tissue is formed and shoots may be induced from callus and subsequently root. Alternatively, somatic embryo formation can be induced in the callus tissue. These somatic embryos germinate as natural embryos to form plants. The culture media will generally contain various amino acids and plant hormones, such as auxin and cytokinins. It is also advantageous to add glutamic acid and proline to the medium, especially for such species as corn and alfalfa. Efficient regeneration will depend on the medium, on the genotype, and on the history of the culture. If these three variables are controlled, then regeneration is usually reproducible and repeatable.

The regenerated plants are transferred to standard soil conditions and cultivated in a conventional manner. The plants are grown and harvested using conventional procedures.

The particular conditions for transformation, selection and regeneration of a plant can be optimized by those of skill in the art. Factors that affect the efficiency of transformation include the species of plant, the target tissue or cell, composition of the culture media, selectable marker genes, kinds of vectors, and light/dark conditions. Therefore, these and other factors may be varied to determine an optimal transformation protocol for any particular plant species. It is recognized that not every species will react in the same manner to the transformation conditions and may require a slightly different modification of the protocols disclosed herein. However, by altering each of the variables, an optimum protocol can be derived for any plant species.

Further, the genetic properties engineered into the transgenic seeds and plants, plant parts, and/or plant cells of the present invention described herein can be passed on by sexual reproduction or vegetative growth and therefore can be maintained and propagated in progeny plants. Generally, maintenance and propagation make use of known agricultural methods developed to fit specific purposes such as harvesting, sowing or tilling.

Accordingly, in some aspects of the invention, a stably transformed plant, plant part and/or plant cell is provided, which comprises in its genome a first heterologous polynucleotide encoding polypeptides having the enzyme activity of glycolate dehydrogenase and a second heterologous polynucleotide encoding a polypeptide having the activity of a CO₂ transporter (e.g., aquaporin), wherein said stably transformed plant or plant regenerated from said stably transformed plant part or plant cell has an increased number of seeds produced per stably transformed plant as compared to a control (e.g., the same plant but which does not comprise in its genome said first and second heterologous polynucleotides). In some embodiments, stably transformed plant, plant part and/or plant cell further comprises in its genome a heterologous polynucleotide encoding a polypeptide having the enzyme activity of a cell wall invertase. In further embodiments, the heterologous polynucleotide encoding a polypeptide having the enzyme activity of a cell wall invertase is overexpressed in said plant, plant part and/or plant cell.

In other embodiments, a stably transformed plant, plant part and/or plant cell is provided, which comprises in its genome, a first heterologous polynucleotide encoding a polypeptide having the activity of a CO₂ transporter (e.g., aquaporin), a second heterologous polynucleotide encoding a polypeptide having the enzyme activity of glyoxylate carboligase and a third heterologous polynucleotide encoding a polypeptide having the enzyme activity of tartronic semialdehyde reductase, wherein said stably transformed plant or plant regenerated from said stably transformed plant part or plant cell has an increased number of seeds produced per stably transformed plant as compared to a control. In some embodiments, stably transformed plant, plant part and/or plant cell further comprises in its genome a heterologous polynucleotide encoding a polypeptide having the enzyme activity of a cell wall invertase. In further embodiments, the heterologous polynucleotide encoding a polypeptide having the enzyme activity of a cell wall invertase is overexpressed in said plant, plant part and/or plant cell.

In some embodiments, a stably transformed plant, plant part and/or plant cell is provided, which comprises in its genome, a first heterologous polynucleotide encoding polypeptides having the enzyme activity of glycolate dehydrogenase, a second heterologous polynucleotide encoding a polypeptide having the activity of a CO₂ transporter (e.g., aquaporin), a third heterologous polynucleotide encoding a polypeptide having the enzyme activity of glyoxylate carboligase and a fourth heterologous polynucleotide encoding a polypeptide having the enzyme activity of tartronic semialdehyde reductase, wherein said stably transformed plant or plant regenerated from said stably transformed plant part or plant cell has an increased number of seeds produced per stably transformed plant as compared to a control. In some embodiments, stably transformed plant, plant part and/or plant cell further comprises in its genome a heterologous polynucleotide encoding a polypeptide having the enzyme activity of a cell wall invertase. In further embodiments, the heterologous polynucleotide encoding a polypeptide having the enzyme activity of a cell wall invertase is overexpressed in said plant, plant part and/or plant cell.

In further embodiments, the present invention provides a stably transformed plant, plant part and/or plant cell is provided, which comprises in its genome a first heterologous polynucleotide encoding polypeptides having the enzyme activity of glycolate dehydrogenase, a second heterologous polynucleotide encoding a polypeptide having the activity of a CO₂ transporter (e.g., aquaporin) and a third heterologous polynucleotide encoding a suppressor of a cell wall invertase inhibitor, wherein said stably transformed plant or plant regenerated from said stably transformed plant part or plant cell has an increased number of seeds produced per stably transformed plant and/or increased assimilate partitioning directed into fruits and seeds and/or increased seed size as compared to a control. In some embodiments, stably transformed plant, plant part and/or plant cell further comprises in its genome a heterologous polynucleotide encoding a polypeptide having the enzyme activity of a cell wall invertase. In further embodiments, the heterologous polynucleotide encoding a polypeptide having the enzyme activity of a cell wall invertase is overexpressed in said plant, plant part and/or plant cell.

In other embodiments, a stably transformed plant, plant part and/or plant cell is provided, which comprises in its genome, a first heterologous polynucleotide encoding a polypeptide having the activity of a CO₂ transporter (e.g., aquaporin), a second heterologous polynucleotide encoding a polypeptide having the enzyme activity of glyoxylate carboligase, a third heterologous polynucleotide encoding a polypeptide having the enzyme activity of tartronic semialdehyde reductase and a fourth heterologous polynucleotide encoding a suppressor of a cell wall invertase inhibitor, wherein said stably transformed plant or plant regenerated from said stably transformed plant part or plant cell has an increased number of seeds produced per stably transformed plant and/or increased assimilate partitioning directed into fruits and seeds and/or increased seed size as compared to a control.

In some embodiments, a stably transformed plant, plant part and/or plant cell is provided, which comprises in its genome, a first heterologous polynucleotide encoding polypeptides having the enzyme activity of glycolate dehydrogenase, a second heterologous polynucleotide encoding a polypeptide having the activity of a CO₂ transporter (e.g., aquaporin), a third heterologous polynucleotide encoding a polypeptide having the enzyme activity of glyoxylate carboligase, a fourth heterologous polynucleotide encoding a polypeptide having the enzyme activity of tartronic semialdehyde reductase and a fifth heterologous polynucleotide encoding a suppressor of a cell wall invertase inhibitor, wherein said stably transformed plant or plant regenerated from said stably transformed plant part or plant cell has an increased number of seeds produced per stably transformed plant and/or increased assimilate partitioning directed into fruits and seeds and/or increased seed size as compared to a control (e.g., the same plant but which does not comprise in its genome said first, second, third, fourth, and fifth heterologous polynucleotides). In some embodiments, stably transformed plant, plant part and/or plant cell further comprises in its genome a heterologous polynucleotide encoding a polypeptide having the enzyme activity of a cell wall invertase. In further embodiments, the heterologous polynucleotide encoding a polypeptide having the enzyme activity of a cell wall invertase is overexpressed in said plant, plant part and/or plant cell.

In some embodiments, a plant, plant part and/or plant cell is provided, which comprises in its genome a modified endogenous cell wall invertase inhibitor gene encoding a polypeptide having reduced or no cell wall invertase inhibitor activity, wherein said plant or plant regenerated from said plant part or plant cell has increased assimilate partitioning directed into fruits and seeds and/or increased seed size as compared to a control (a plant that does not comprise said modified cell wall invertase inhibitor gene).

In other embodiments, a plant, plant part and/or plant cell is provided, which comprises in its genome a modified endogenous cell wall invertase inhibitor gene encoding a polypeptide having reduced or no cell wall invertase inhibitor activity and a heterologous polynucleotide encoding polypeptides having the enzyme activity of glycolate dehydrogenase, wherein said plant or plant regenerated from said plant part or plant cell has increased seed number, and increased assimilate partitioning directed into fruits and seeds and/or increased seed size as compared to a control (a plant that does not comprise said modified cell wall invertase inhibitor gene and said heterologous polynucleotide encoding polypeptides having the enzyme activity of glycolate dehydrogenase).

In still other embodiments, a plant, plant part and/or plant cell is provided, which comprises in its genome a modified endogenous cell wall invertase inhibitor gene encoding a polypeptide having reduced or no cell wall invertase inhibitor activity, a first heterologous polynucleotide encoding polypeptides having the enzyme activity of glycolate dehydrogenase, and a second heterologous polynucleotide encoding a polypeptide having the enzyme activity of tartronic semialdehyde reductase, a third heterologous polynucleotide encoding a polypeptide having the enzyme activity of glyoxylate carboligase, wherein said plant or plant regenerated from said plant part or plant cell has increased seed number, and increased assimilate partitioning directed into fruits and seeds and/or increased seed size as compared to a control (a plant that does not comprise said modified cell wall invertase inhibitor gene and said first, second and third heterologous polynucleotides).

Additionally provided herein are seeds produced from a plant of the invention, wherein said seeds comprise in their genomes one or more of the heterologous polynucleotides of the invention (e.g., a polynucleotide encoding a polypeptide having the enzyme activity of glycolate dehydrogenase, polynucleotide encoding a polypeptide having the enzyme activity of a tartronic semialdehyde reductase, a polynucleotide encoding a polypeptide having the enzyme activity of a glyoxylate carboligase, a polynucleotide encoding a polypeptide having the activity of a cell wall invertase, a polynucleotide encoding a polypeptide having the activity of a suppressor of a cell wall invertase inhibitor, and/or a polynucleotide encoding a polypeptide having the activity of a CO₂ transporter) and/or a modified cell wall invertase gene as described herein.

Additionally, crops comprising a plurality of the transgenic plants of the invention are provided. Nonlimiting examples of types of crops comprising a plurality of transgenic plants of the invention include an agricultural field, a golf course, a residential lawn, a road side, an athletic field, and/or a recreational field.

The present invention further provides a product or products produced from the stably transformed plant, plant cell or plant part of the invention. In particular embodiments, the present invention further provides a product produced from the seed of the stably transformed plant.

In some aspects of the invention, a product can be a product harvested from the transgenic plants, plant parts, plant cells, and/or progeny thereof, or crops of the invention, as well as a processed product produced from said harvested product. A harvested product can be a whole plant or any plant part, as described herein, wherein said harvested product comprises a heterologous polynucleotide of the invention. Non-limiting examples of a harvested product include a seed, a fruit, a flower or part thereof (e.g., an anther, a stigma, and the like), a leaf, a stem, and the like. In some embodiments, a processed product includes, but is not limited to, a flour, meal, oil, starch, cereal, and the like produced from a harvested seed of the invention. In some embodiments, the product produced from the stably transformed plants, plant parts and/or plant cells can include, but is not limited to, biofuel, food, drink, animal feed, fiber, commodity chemicals, cosmetics, and/or pharmaceuticals.

As used herein, the terms “nucleic acid,” “nucleic acid molecule,” “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “oligonucleotide” and “polynucleotide” are also used interchangeably herein to refer to a heteropolymer of nucleotides. Nucleic acid sequences provided herein are presented herein in the 5′ to 3′ direction, from left to right and are represented using the standard code for representing the nucleotide characters as set forth in the U.S. sequence rules, 37 CFR §§1.821-1.825 and the World Intellectual Property Organization (WIPO) Standard ST.25.

As used herein, the term “gene” refers to a nucleic acid molecule capable of being used to produce mRNA, antisense RNA, miRNA, and the like. Genes may or may not be capable of being used to produce a functional protein. Genes can include both coding and non-coding regions (e.g., introns, regulatory elements, promoters, enhancers, termination sequences and 5′ and 3′ untranslated regions). A gene may be “isolated” by which is meant a nucleic acid molecule that is substantially or essentially free from components normally found in association with the nucleic acid molecule in its natural state. Such components include other cellular material, culture medium from recombinant production, and/or various chemicals used in chemically synthesizing the nucleic acid molecule.

As used herein, the terms “fragment” when used in reference to a polynucleotide will be understood to mean a nucleic acid molecule or polynucleotide of reduced length relative to a reference nucleic acid molecule or polynucleotide and comprising, consisting essentially of and/or consisting of a nucleotide sequence of contiguous nucleotides identical or almost identical (e.g., 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% identical) to the reference nucleic acid or nucleotide sequence. Thus, for example, a functional fragment of a suppressor of a cell wall invertase inhibitor is a fragment that retains at least 50% or more of the ability to suppress a cell wall invertase inhibitor. A nucleic acid fragment according to the invention may be, where appropriate, included in a larger polynucleotide of which it is a constituent.

As used herein, a “functional” polypeptide or “functional fragment” is one that substantially retains at least one biological activity normally associated with that polypeptide. In particular embodiments, the “functional” polypeptide or “functional fragment” substantially retains all of the activities possessed by the unmodified peptide. By “substantially retains” biological activity, it is meant that the polypeptide retains at least about, e.g., 50%, 55%, 60%, 65%, 70%, 75%, 80%, 85%, 90%, 91%, 92%, 93%, 94%, 95%, 96%, 97%, 98%, 99%, or more, of the biological activity of the native polypeptide (and can even have a higher level of activity than the native polypeptide). A “non-functional” polypeptide is one that exhibits little or essentially no detectable biological activity normally associated with the polypeptide (e.g., at most, only an insignificant amount, e.g., less than about 10% or even 5%). Thus, for example, a functional fragment of a cwI, GDH, TSR, GLC or a CO₂ transporter polypeptide is a polypeptide that retains at least 50% or more cwI, GDH, TSR, GLC or CO₂ transporter activity or functionality.

Thus, for example, a functional fragment of glycolate dehydrogenase, which converts glycolate to glyoxylate, is a fragment that can convert glycolate to glyoxylate at a rate of 50% or more when compared to the activity of the native polypeptide. In other embodiments, a functional fragment of glyoxylate carboligase, which converts glyoxylate into tartronic-semialdehyde, is a fragment that can convert glyoxylate to tartronic-semialdehyde at a rate of 50% or more when compared to the activity of the native polypeptide. In still further embodiments, a functional fragment of tartronic semialdehyde reductase, which reduces tartronic-semialdehyde into glycerate, is a fragment that can reduce tartronic-semialdehyde into glycerate at a rate of 50% or more when compared to the activity of the native polypeptide. In a further embodiment, a functional fragment of a cell wall invertase is a fragment that can direct assimilate partitioning into fruits and/or seeds at a rate of 50% or more when compared to the activity of the native polypeptide. In other embodiments, a functional fragment of a CO₂ transporter, which improves the rate of photosynthesis, is a fragment that can improve the rate of photosynthesis by at least a rate of 50% as compared to the native polypeptide.

An “isolated” nucleic acid molecule or nucleotide sequence or nucleic acid construct or double stranded RNA molecule of the present invention is generally free of nucleotide sequences that flank the nucleic acid of interest in the genomic DNA of the organism from which the nucleic acid was derived (such as coding sequences present at the 5′ or 3′ ends). However, the nucleic acid molecule of this invention can include some additional bases or moieties that do not deleteriously or materially affect the basic structural and/or functional characteristics of the nucleic acid molecule.

Thus, an “isolated nucleic acid molecule” or “isolated nucleotide sequence” is a nucleic acid molecule or nucleotide sequence that is not immediately contiguous with nucleotide sequences with which it is immediately contiguous (one on the 5′ end and one on the 3′ end) in the naturally occurring genome of the organism from which it is derived. Accordingly, in one embodiment, an isolated nucleic acid includes some or all of the 5′ non-coding (e.g., promoter) sequences that are immediately contiguous to a coding sequence. The term therefore includes, for example, a recombinant or heterologous nucleic acid that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant nucleic acid that is part of a hybrid nucleic acid molecule encoding an additional polypeptide or peptide sequence.

The term “isolated” can further refer to a nucleic acid molecule, nucleotide sequence, polypeptide, peptide or fragment that is substantially free of cellular material, viral material, and/or culture medium (e.g., when produced by recombinant DNA techniques), or chemical precursors or other chemicals (e.g., when chemically synthesized). Moreover, an “isolated fragment” is a fragment of a nucleic acid molecule, nucleotide sequence or polypeptide that is not naturally occurring as a fragment and would not be found as such in the natural state. “Isolated” does not mean that the preparation is technically pure (homogeneous), but it is sufficiently pure to provide the polypeptide or nucleic acid in a form in which it can be used for the intended purpose. In representative embodiments of the invention, an “isolated” nucleic acid molecule, nucleotide sequence, and/or polypeptide is at least about, e.g., 5%, 10%, 15%, 20%, 25%, 30%, 40%, 50%, 60%, 70%, 75%, 80%, 85%, 90%, 95%, 97%, 98%, 99% pure (w/w) or more. In other embodiments, an “isolated” nucleic acid, nucleotide sequence, and/or polypeptide indicates that at least about, e.g., a 5-fold, 10-fold, 25-fold, 100-fold, 1000-fold, 10,000-fold, 100,000-fold or more enrichment of the nucleic acid (w/w) is achieved as compared with the starting material.

As used herein, “complementary” polynucleotides are those that are capable of hybridizing via base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. For example, the sequence “A-G-T” binds to the complementary sequence “T-C-A.” It is understood that two polynucleotides may hybridize to each other even if they are not completely or fully complementary to each other, provided that each has at least one region that is substantially complementary to the other.

The terms “complementary” or “complementarity,” as used herein, refer to the natural binding of polynucleotides under permissive salt and temperature conditions by base-pairing. Complementarity between two single-stranded molecules may be “partial,” in which only some of the nucleotides bind, or it may be complete when total complementarity exists between the single stranded molecules either along the full length of the molecules or along a portion or region of the single stranded molecules. The degree of complementarity between nucleic acid strands has significant effects on the efficiency and strength of hybridization between nucleic acid strands.

As used herein, the terms “substantially complementary” or “partially complementary” mean that two nucleic acid sequences are complementary at least at about 50%, 60%, 70%, 80% or 90% of their nucleotides. In some embodiments, the two nucleic acid sequences can be complementary at least at about 85%, 90%, 95%, 96%, 97%, 98%, 99% or more of their nucleotides. The terms “substantially complementary” and “partially complementary” can also mean that two nucleic acid sequences can hybridize under high stringency conditions and such conditions are well known in the art.

As used herein, “heterologous” refers to a nucleic acid molecule or nucleotide sequence that either originates from another species or is from the same species or organism but is modified from either its original form or the form primarily expressed in the cell. Thus, a nucleotide sequence derived from an organism or species different from that of the cell into which the nucleotide sequence is introduced, is heterologous with respect to that cell and the cell's descendants. In addition, a heterologous polynucleotide includes a nucleotide sequence derived from and inserted into the same natural, original cell type, but which is present in a non-natural state, e.g. present in a different copy number, and/or under the control of different regulatory sequences than that found in the native state of the nucleic acid molecule.

As used herein, the terms “transformed” and “transgenic” refer to any plant, plant part, and/or plant cell that contains all or part of at least one recombinant (e.g., heterologous) polynucleotide. In some embodiments, all or part of the recombinant polynucleotide is stably integrated into a chromosome or stable extra-chromosomal element, so that it is passed on to successive generations. For the purposes of the invention, the term “recombinant polynucleotide” refers to a polynucleotide that has been altered, rearranged, or modified by genetic engineering. Examples include any cloned polynucleotide, or polynucleotides, that are linked or joined to heterologous sequences. The term “recombinant” does not refer to alterations of polynucleotides that result from naturally occurring events, such as spontaneous mutations, or from non-spontaneous mutagenesis followed by selective breeding.

The term “transgene” as used herein, refers to any nucleotide sequence used in the transformation of an organism. Thus, a transgene can be a coding sequence, a non-coding sequence, a cDNA, a gene or fragment or portion thereof, a genomic sequence, a regulatory element and the like. A “transgenic” organism, such as a transgenic plant, transgenic yeast, or transgenic bacterium, is an organism into which a transgene has been delivered or introduced and the transgene can be expressed in the transgenic organism to produce a product, the presence of which can impart an effect and/or a phenotype in the organism.

Different nucleotide sequences or polypeptide sequences having homology are referred to herein as “homologues.” The term homologue includes homologous sequences from the same and other species and orthologous sequences from the same and other species. “Homology” refers to the level of similarity between two or more nucleotide sequences and/or amino acid sequences in terms of percent of positional identity (i.e., sequence similarity or identity). Homology also refers to the concept of similar functional properties among different nucleic acids, amino acids, and/or proteins.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “substantially identical” means that two nucleotide sequences have at least 70%, 75%, 80%, 85%, 90% or 95% sequence identity. In some embodiments, the two nucleotide sequences can have at least 80%, 85%, 90%, 91%, 92%, 93%, 94%, 95%, 96%, 97%, 98%, 99% or 100% sequence identity. Thus, for example, a homolog of a polynucleotide of the invention can have at least about, e.g., 80%, 85%, 90%, 91%, 92%, 93%, 94%, 95%, 96%, 97%, 98%, 99% or 100% sequence identity to, for example, a heterologous polynucleotide encoding polypeptides having the enzyme activity of glycolate dehydrogenase, a heterologous polynucleotide encoding a polypeptide having the activity of a CO₂ transporter, and/or a heterologous polynucleotide encoding a suppressor of cwII.

Two nucleotide sequences can also be considered to be substantially complementary when the two sequences hybridize to each other under stringent conditions. In some representative embodiments, two nucleotide sequences considered to be substantially complementary hybridize to each other under highly stringent conditions.

A nonlimiting example of “stringent” hybridization conditions include conditions represented by a wash stringency of 50% formamide with 5×Denhardt's solution, 0.5% SDS and 1×SSPE at 42° C. “Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). In some representative embodiments, two nucleotide sequences considered to be substantially identical hybridize to each other under highly stringent conditions. Generally, highly stringent hybridization and wash conditions are selected to be about 5° C. lower than the thermal melting point (Tₘ) for the specific sequence at a defined ionic strength and pH.

An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). In some embodiments, “percent identity” can refer to the percentage of identical amino acids in an amino acid sequence.

Optimal alignment of sequences for aligning a comparison window is well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., Burlington, Mass.). The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

The percent of sequence identity can be determined using the “Best Fit” or “Gap” program of the Sequence Analysis Software Package™ (Version 10; Genetics Computer Group, Inc., Madison, Wis.). “Gap” utilizes the algorithm of Needleman and Wunsch (Needleman and Wunsch, J Mol. Biol. 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, Adv. Appl. Math., 2:482-489, 1981, Smith et al., Nucleic Acids Res. 11:2205-2220, 1983).

Useful methods for determining sequence identity are also disclosed in Guide to Huge Computers (Martin J. Bishop, ed., Academic Press, San Diego (1994)), and Carillo et al. (Applied Math 48:1073(1988)). More particularly, preferred computer programs for determining sequence identity include but are not limited to the Basic Local Alignment Search Tool (BLAST) programs which are publicly available from National Center Biotechnology Information (e.g., NCBI) at the National Library of Medicine, National Institute of Health, Bethesda, Md. 20894; see BLAST Manual, Altschul et al., e.g., NCBI, NLM, NIH; (Altschul et al., J Mol. Biol. 215:403-410 (1990)); version 2.0 or higher of BLAST programs allows the introduction of gaps (deletions and insertions) into alignments; for peptide sequence BLASTX can be used to determine sequence identity; and for polynucleotide sequence BLASTN can be used to determine sequence identity.

Accordingly, the present invention further provides polynucleotides having substantial sequence identity (e.g., 70%, 75%, 80%, 81%, 82%, 83%, 84%, 85%, 86%, 87%, 88%, 89%, 90%, 91%, 92%, 93%, 94%, 95%, 96%, 97%, 98%, 99% and/or 100% identity) to a polynucleotide of the present invention (e.g., a heterologous polynucleotide encoding polypeptides having the enzyme activity of glycolate dehydrogenase; a heterologous polynucleotide encoding a polypeptide having the activity a CO₂ transporter; a heterologous polynucleotide encoding a polypeptide having the enzyme activity of tartronic semialdehyde reductase, a heterologous polynucleotide encoding a polypeptide having the enzyme activity of glyoxylate carboligase, a heterologous polynucleotide encoding a polypeptide having the enzyme activity of a cell wall invertase, and/or a heterologous polynucleotide encoding a suppressor of an inhibitor of cell wall invertase).

The following examples are not intended to be a detailed catalog of all the different ways in which the present invention may be implemented or of all the features that may be added to the present invention. Persons skilled in the art will appreciate that numerous variations and additions to the various embodiments may be made without departing from the present invention. Hence, the following descriptions are intended to illustrate some particular embodiments of the invention, and not to exhaustively specify all permutations, combinations and variations thereof.

### EXAMPLES

The oilseed crop Camelina sativa (L.) Crantz has been naturalized to almost all of the United States (United States Depai talent of Agriculture USDA, N.R.C.S. Plant Database. 2011). It is grown in rotation either as an annual summer crop or biannual winter crop. It is adapted to a wide range of temperate climates on marginal land, is drought and salt tolerant, and requires very little water or fertilizer. Its seeds have a high oil content (≧40%) that can be extracted by energy efficient cold pressing. The remaining omega-3 fatty acid-rich meal has been approved by the FDA for inclusion in livestock feed. A further advantage is that camelina does not compete for land with food crops and produces feed for livestock as well as productivity (and jobs) on unarmed land. Camelina further has a short life cycle and can produce up to four generations per year in greenhouses.

Camelina sativa is genetically engineered to express glycolate dehydrogenase and aquaporin (e.g., a CO₂ transporter) and optionally tartronic semialdehyde reductase, glyoxylate carboligase, cell wall invertase and/or a suppressor of a cell wall invertase inhibitor.

### Example 1

### Transformation of Camelina

Camelina sativa variety (Ukraine) is used and Agrobacterium-mediated transformation is used for transformation. Camelina can be transformed by “floral dip” or vacuum application (Lu and Kang. Plant Cell Reports 27(2):273-278 (2008); Liu et al. In Vitro Cell Devel Biol-Animal. 44:S40-S41 (2008)) or any other method effective for the generation of stable camelina transformants. The Gateway vector with CaMV 35S promoter (Earley et al. Plant Journal. 45(4):616-629 (2006)) can be used for construction of the transgene cassettes. Gateway vectors or other vectors can be used for expression in seed, seed coat, or seed pod with the respective tissue specific promoter and/or targeting sequences.

To facilitate selection of seedlings after transformation of camelina, a selectable marker gene will be used together with a transgene. Thus, for each expression cassette, hygromycin B, bialaphos/ppt, eGFP or mCherry selection (Lu and Kang. Plant Cell Reports 27(2):273-278 (2008)) can be used to facilitate selection of crossed seeds or seedlings between two clusters of genes. Double selection can be performed, followed by polymerase chain reaction (PCR) assays for each transgene to ensure the presence of the transgenes. Transgene expression can be monitored by Western and/or quantitative reverse transcriptase (qRT)-PCR, and validated by Northern blot analysis. Thus, four selectable markers will be used in selection from multiple crosses.

### Generating Homozygous Transgenic Lines

After “floral dip” transformation, about 1% of the seeds will be transgenic, and can be identified by selection. As discussed above, four different selectable marker genes will be evaluated: HPT, BAR, eGFP and mCherry. After the selfing of the T1 plants, the seeds produced are the T2 generation. T2 plants should segregate to have ¼ homozygous for the transgene, ½ heterozygous for the transgene, and ¼ without transgene. Selection will be carried out on the T3 generation to identify homozygotes. The seeds of the lines from the T3 generation will be multiplied.

### Other Transgenic Plants

In some case, plants can be evaluated as heterozygotes. For plants from crosses, we will identify plants with desirable combinations of transgenes by double, triple or quadruple selection.

### Protocol for Transforming Camelina

Luria Broth (LB) medium for growing Agrobacterium

### Infiltration Medium:

½×MS salts
5% (w/v)Sucrose
0.044 uM BAP
0.05% Silwet L-77

### Procedure:

(1) Two days prior to transformation, a pre-culture of Agrobacterium carrying the appropriate binary vector is prepared by inoculating the Agrobacterium onto 3 ml LB medium including suitable antibiotics and incubating the culture at 28° C.
(2) One day prior to transformation a larger volume of (150 ml-300 ml) LB medium is inoculated with at least 1 ml of the preculture and incubated at 28° C. for about 16-24 hrs.
(3) Water plants prior to transformation.
(4) On the day of transformation of the plant, Agrobacterium cells are pelleted by centrifugation at 6000 rpm for 10 min at room temperature (e.g., about 19° C. to about 24° C.).
(5) The pellet is resuspended in 300-600 ml of infiltration medium (note: the infiltration medium is about double the volume used in the agro culture (about 150-300 ml)).
(6) The suspension solution is transferred to an open container that can hold the volume of infiltration medium prepared (300-600 ml) in which plants can be dipped and which fits into a desiccator.
(7) Place the container from (6) into a desiccator, invert a plant and dip the inflorescence shoots into the infiltration medium.
(8) Connect the desiccator to a vacuum pump and evacuate for 5 min at 16-85 kPa.
(9) Release the vacuum slowly.
(10) After releasing vacuum, remove the plants and orient them into an upright position or on their sides in a plastic nursery flat, and place a cover over them for the next 24 hours to maintain humid conditions.
(11) The next day, the cover is removed, the plants rinsed with water and returned to their normal growing conditions (e.g., of about 22° C./18° C. (day/night) with daily watering under about 250-400 μE white light).
(12) A week later the plants were transformed again, repeating steps 1-11.
(13) The plants were watered on alternate days beginning after transformation for about 2-3 weeks and then twice a week for about another 2 weeks after which they were watered about once a week for about another 2-3 weeks for drying.

### Example 2

### Analysis of Transformed C. sativa Plants

(1) Verification of expression in the various plant organelles
RT-PCR and pRT-PCR Methods.

RNA is isolated using the RNeasy kit (Qiagen), with an additional DNase I treatment to remove contaminating genomic DNA. Reverse transcription (RT) was carried out to generate cDNA using Omniscript reverse transcriptase enzyme (Qiagen). Quantitative RT-PCR was carried out using Full Velocity SYBR-Green® QPCR Master Mix (Stratagene) on a MX3000P thermocycler (Stratagene). Gene specific primers for select genes were designed with the help of AtRTPrimer, a database for generating specific RT-PCR primer pairs (Han and Kim, BMC Bioinformatics 7:179 (2006)). Relative gene expression data were generated using the 2^{-ΔΔCt} method (Livak and Schmittgen, Methods 25:402-408 (2001)) using the wild-type zero time point as the reference. PCR conditions were 1 cycle of 95° C. for 10 min, 95° C. for 15 s, and 60° C. for 30 s to see the dissociation curve, 40 cycles of 95° C. for 1 minute for DNA denaturation, and 55° C. for 30 s for DNA annealing and extension.

### Example 3

### Expression of GDH, TSR and GCL in Camelina sativa

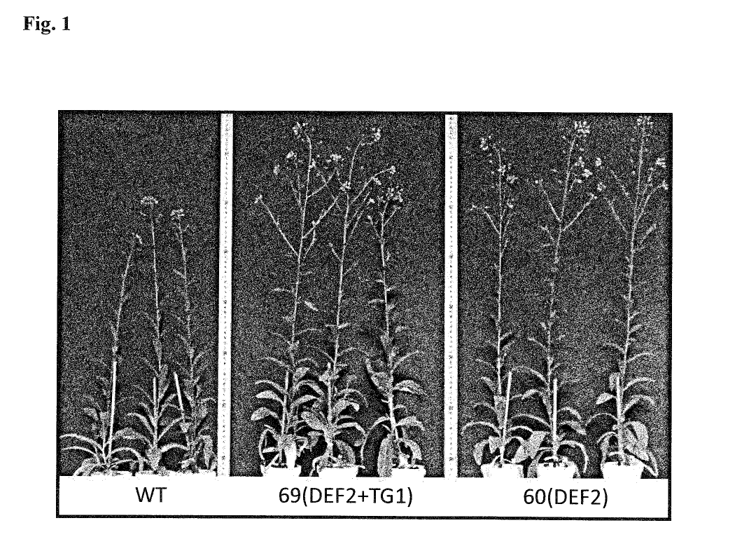

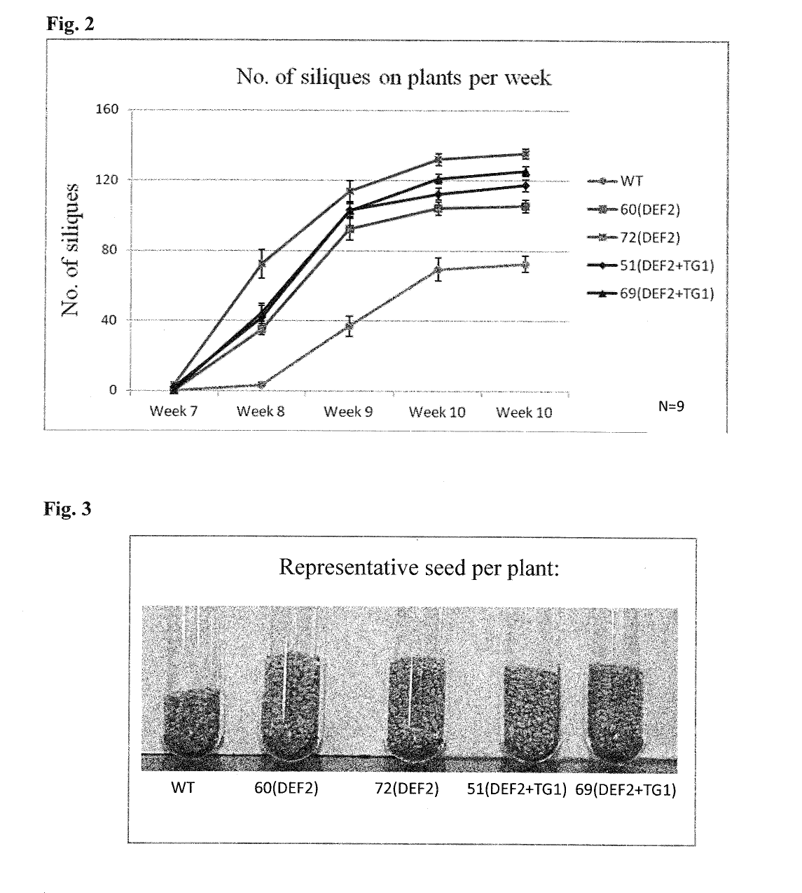

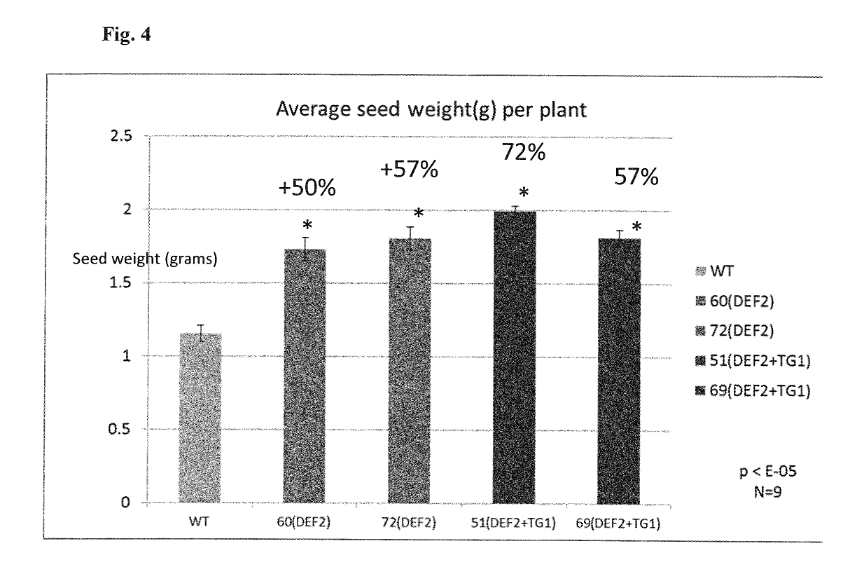

The nucleotide sequences encoding the three subunits of glycolate dehydrogenase from Escherichia coli (glcD, glcE and glcF) were transformed into camelina as described herein and transgenic plants obtained. In addition, plants were engineered to express polynucleotides encoding tartronic semialdehyde reductase (TSR) and glyoxylate carboligase (GCL) (TSR+GCL=TG1). T4 plants were grown side by side with WT plants in short day (9 hour day/15 hour night) conditions and analyzed for various phenotypes. At five weeks of age, at least eight independent insertion lines expressing glycolate dehydrogenase subunits show increase in height over WT plants of the same age. Additionally, at five week of age, at least nine independent insertion lines expressing glycolate dehydrogenase, tartronic semialdehyde reductase and glyoxylate carboligase show increase in height over WT plants of the same age. The transgenic lines were determined to have an earlier floral induction (FIG. 1), increased number of silique (seed capsule) formation per week (FIG. 2), increased seed yield (FIG. 3 and FIG. 4). Transgenic plants expressing glycolate dehydrogenase show increase in seed yield by about 50% over WT. Transgenic plants expressing glycolate dehydrogenase, tartronic semialdehyde reductase and glycolate carboligase show increase in yields of up to 72% over WT.

### Example 4

### Expression of Aquaporin in Camelina sativa

Plants were transformed with aquaporin from tobacco (NtAQP1) (Uehlein et al. Nature. 425(6959):734-7 (2003); Uehlein et al. Plant Cell 20(3):648-57 (2008); Flexas et al. Plant J. 48(3):427-39 (2006)). This NtAQP1 is localized to the inner chloroplast envelope membrane as well as to mesophyll cell plasma membranes (Uehlein et al. Plant Cell 20(3):648-57 (2008)). The construct used for transformation comprised a 35S CaMV promoter operably linked to the polynucleotide encoding tobacco aquaporin (e.g., SEQ ID NO:23; GENBANK Accession No: AJ001416.1), which is further operably linked to a polynucleotide encoding green fluorescent protein (GFP).

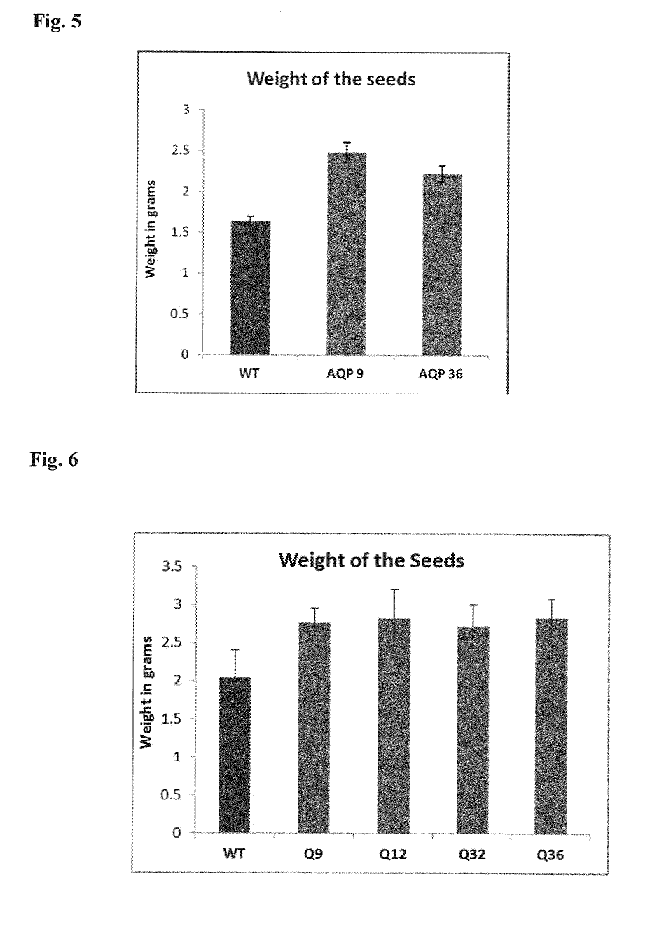

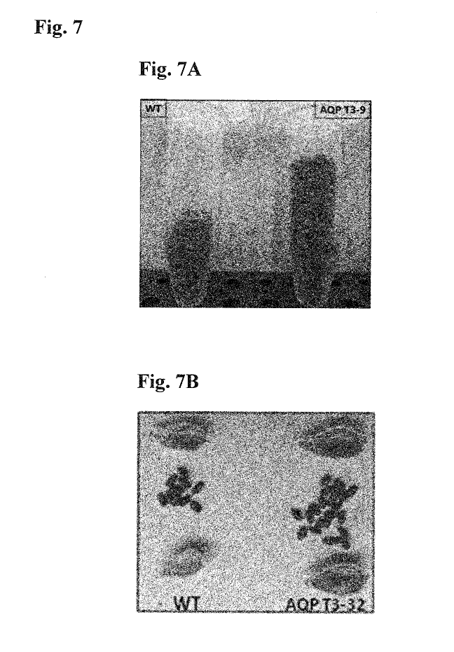

Analysis of camelina transformants expressing tobacco aquaporin (e.g., SEQ ID NO:20) showed that the transformation resulted in plants with increased rates of photosynthesis and an increased number of seeds per silique (FIGS. 5, 6, and 7)

### Example 5

### Expression of GDH, TSR, GLC and Aquaporin in Camelina sativa

Camelina plants were transformed with the three subunits of glycolate dehydrogenase. In one approach, the three subunits were introduced into the plant nuclear genome as one polypeptide with ubiquitin cleavage sites between them (Walker et al. 2007 Plant Biotechnology Journal 5:413-421). In this case, the polynucleotides were driven by a 35S promoter. In another approach, the GlcD, GlcE and GlcF polynucleotides were driven by separate constitutive promoters (e.g., tobacco EntCUP4 (Malik et al. 2002 Theor Appl Genet 105:505-514), CamV 35S (Odell et al. (1985) Nature 313:810-812) and/or the Arabidopsis Acting promoter (Yong-Qiang An et al. 1996 The Plant Journal 10(1):107-121; Yong-Qiang An et al. 2010 Plant Mol Bio Rep 28:481-490)). In the latter case, the three polynucleotides were cloned in the multiple cloning site of the same binary vector, namely pCAMBIA2300-mCherry (DEF2 plants). Camelina plants were transformed with tartronic semialdehyde reductase (TSR) and glyoxylate carboligase (GCL). The tartronic semialdehyde reductase (TSR) and the glyoxylate carboligase (GCL) polynucleotides each were expressed under the control of separate 35S promoters (Odell et al. (1985) Nature 313:810-812), but cloned into the T-DNA of the same binary vector, namely pEG100 (TG1 plants). Each of the five genes, namely GlcD, GlcE, GlcF, GCL and TSR were targeted to expression in the chloroplasts by fusing a chloroplast transit peptide to the 5′ end of the coding sequence. The chloroplast transit peptide of Arabidopsis rubisco small subnunit (RBCS) (Lee et al. 2008, Plant Cell 20: 1603-1622) was used for chloroplast targeting of GlcD, GlcF and TSR. The chloroplast transit peptide of biotin carboxyl carrier protein (BCCP) (Lee et al. 2008, Plant Cell 20: 1603-1622) was used for chloroplast targeting of GlcE and GCL. In this particular example, Camelina plants were cotransformed with the T-DNA containing all three subunits of glycolate dehydrogenase construct (DEF2) and the T-DNA containing tartronic semialdehyde reductase and the glyoxylate carboxyligase polynucleotides (TG1). These plants expressed all the five polynucleotides (glcD, glcE and glcF from DEF2+SR and GCL from TG1). The data from these constructs has been presented in FIGS. 1-4.

Plants were grown under short-day (9 h light/15 h dark) conditions. Light was set at 430 μmol m⁻² s⁻¹ and the temperature was set at 22° C. at both day and night time. Relative humidity was 48-50%. The growth phenotypes of plants were monitored and photographed weekly. The plants in FIG. 1 are seven-week-old, and show representative increases in growth of transgenic plants (expressing bypass genes) over WT. Once the plants started making siliques, the numbers of siliques formed per plant were counted per week. FIG. 2 shows the average number of siliques (number of plants=9) formed by transgenic plants and WT plants. The transgenic plants have a faster rate of silique development than WT. FIG. 3 shows representative seed yield from transgenic vs. WT plants. The total seed harvested from one plant of each transgenic line were photographed. The average seed yield (number of plants=9) from transgenics and WT plants are depicted in FIG. 4. Transgenic plants have a 50-72% higher seed yield than WT.

### Example 6

### Promoters of Cell Wall Invertase Inhibitor and Tissue Preferred Expression

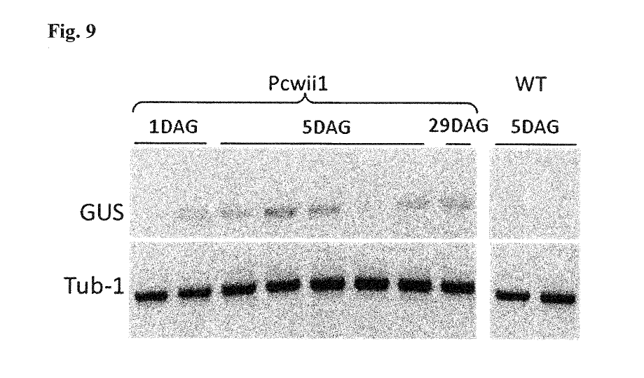

Camelina plants were transformed with a construct in which the GUS reporter gene was operably linked to the camelina cwII1 or the camelina cwII2 promoter. Transgenic plants resulting from the transformation were analyzed for GUS expression patterns. Gene specific primers for the GUS gene were designed using PerlPrimer open-source software. RNA extraction was performed using TRIzol® (Life Technologies) according to manufacturer's instructions. The RNA to cDNA EcoDry™ Premix kit (Clontech) was used according to manufacturer's instructions was used for cDNA synthesis. PCR conditions were 1 cycle of 95° C. for 10 min, and 28 cycles of 95° C. for 15 s, 60° C. for primer annealing, and 68° C. for 30 s for DNA extension, and one cycle of 68° C. for 7 min for final DNA extension. Plants transformed with the cwII1 promoter showed GUS transcript at different plant ages (FIG. 9).

Tissues from plants spanning developmental stages were stained for GUS expression in a protocol modified from Link et al (2004). Briefly, tissues from T2 plants were harvested directly into phosphate-buffered staining solution containing 1 mg/mL X-Gluc and incubated for 24 or 48 hours. Samples were washed in 70% EtOH until all chlorophyll was removed and analyzed for localized blue coloration. When under the control of the cwII1 promoter, GUS was strongly visible throughout the vasculature of plants and tissues at a range of developmental stages and maturation (FIG. 9). When under the control of the cwII2 promoter, GUS was visible at the root tip and in the stele of developing seed embryos (FIG. 10).

### Example 7

### Increasing Assimilate Partitioning into Seeds

Camelina has also been engineered to increase the export of the assimilated carbon from the leaves to the fruits and seeds via introduction into the plant of a suppressor of cwII, RNAi. The export of sugars occurs from photosynthesizing mesophyll cells through the cell wall into the phloem/companion cell complex, which carries sugars via mass flow to non-photosynthetic tissues. Phloem unloading occurs either via the cell wall (apoplasm) or via plasmodesmata (Koch, K., Curr Opin Plant Biol. 7(3):235-46 (2004); Ward et al. International Review of Cytology—a Survey of Cell Biology Vol 178:41-71 (1998)). Export and import through the apoplasm are controlled by the activity of cell wall invertase (cwI), which hydrolyzes sucrose into glucose and fructose and is regulated by a specific inhibitor protein (cwII) (Ward et al. International Review of Cytology—a Survey of Cell Biology Vol 178:41-71 (1998); Ruan et al. Molecular Plant. 3(6):942-955 (2010)). In general, low cell wall invertase activity increases sucrose export from the source tissue, and high cell wall invertase activity increases sucrose unloading into fruits and seeds/grains. Quantitative trait loci analysis for fruit size in tomato (Lin5), and grain size in rice (GIF1) and maize (MN1) identified mutations in cell-wall invertases that led to reduction in its activity in pedicel/fruit tissues (Wang et al. Nature Genetics 40(11):1370-1374 (2008); Fridman et al. Science. 305(5691):1786-1789 (2004); Cheng et al. Plant Cell. 8(6):971-983 (1996)) as key regulators for phloem unloading and therefore determinants of seed and fruit size. Fruit-specific suppression of the cell wall invertase inhibitor (cwII) in tomato and cell wall invertase (cwI) over-expression in rice led to increases in net seed/grain weight of 22% and 10%, respectively (Wang et al. Nature Genetics 40(11):1370-1374 (2008); Jin et al. Plant Cell. 21(7):2072-89 (2009). Two general approaches have been used to modify sucrose flux: overexpression of cwI or repression of its inhibitor protein cwII (Wang et al. Nature Genetics 40(11):1370-1374 (2008); Sonnewald et al. Plant J. 1(1):95-106 (1991); von Schaewen et al. Embo J 9(10):3033-44 (1990); Zanor et al. Plant Physiology 150(3):1204-1218 (2009); Jin et al. Plant Cell. 21(7):2072-89 (2009); Greiner et al. Nat Biotechnol. 17(7):708-11 (1999)).

In the present invention, suppression of CwII in camelina via RNAi technology is used to direct assimilate partitioning into fruit/seeds and/or increased seed size. The nucleotide sequence encoding camelina CWII1, including promoter and coding sequence, and the nucleotide sequence encoding camelina CWII2 including promoter and coding sequence, are shown below (SEQ ID NO:25, and SEQ ID NO:26, respectively).

These promoter sequences (SEQ ID NO:27 (cwII1); SEQ ID NO:28 (cwII2)) can be used in fusion constructs with RNAi to cwII to inhibit cwII. Thus, for example, a fusion construct between the nucleotide sequences of SEQ ID NO:27 and SEQ ID NO:22 and/or between the nucleotide sequences of SEQ ID NO:28 and SEQ ID NO:23 can be constructed and used to inhibit cwII. Additionally, an RNAi construct of this invention for inhibition of cwII can include a fusion between the nucleotide sequences of SEQ ID NO:27 and SEQ ID NO:24 and/or between the nucleotide sequences of SEQ ID NO:28 and SEQ ID NO:24.

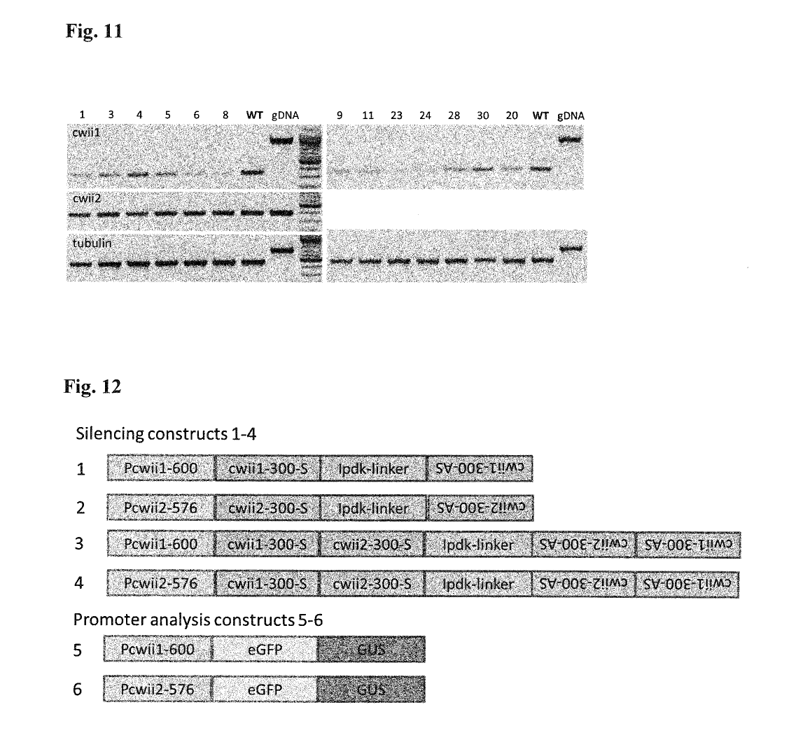

Plants were transformed with a fusion construct between an RNAi hairpin for the silencing of cwII1 expression and the cwII1 promoter (e.g., SEQ ID NO:27 (cwII1) and SEQ ID NO:22 (cwII1 RNAi)). Analysis of the transformed plants showed that the transformation produced reduced transcript abundance of cwII1, as compared to wild type, without affecting transcript abundance of cwII2 (FIG. 11). Expression of the tubulin gene (Tub-1) is provided as a control.

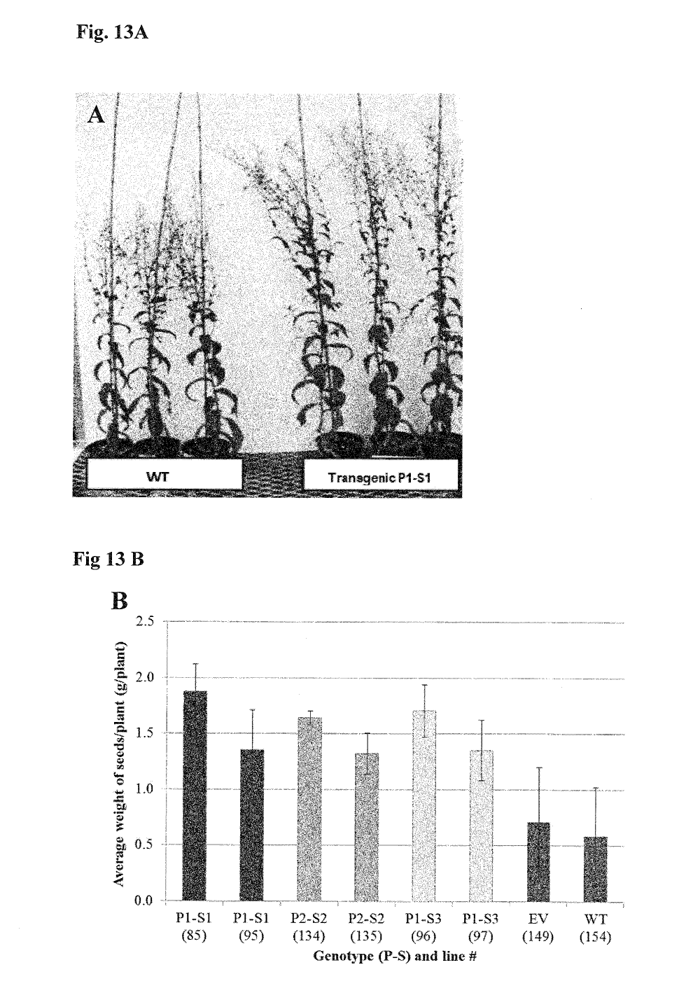

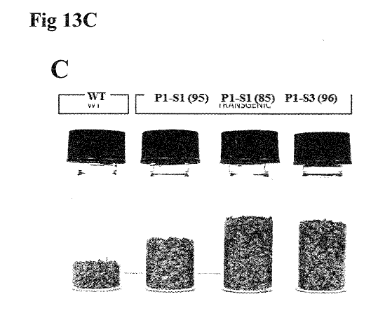

Cell wall invertase inhibitor: We have shown that Camelina sativa has two cell wall invertase inhibitors (CsCWII-1 and CsCwII-2) that differ in their local expression pattern. We generated transgenic Camelina plants with reduced transcript and protein levels of either or both CwII genes using an artificial miRNA technology. The endogenous promoters for these genes, pCWII-1 (P1) and pCWII-2 (P2), were used to drive expression of isoform-specific artificial miRNA constructs CwII-1 (S1), CwII-2 (S2), or both (S3), either against their respective CWII transcripts (P1-S1; P2-S2) or against both constructs (P1-S3) (FIG. 12). All three constructs repressing CwIIs were effective in reducing the respective mRNAs and led to increased vegetative biomass production (FIG. 13A) and higher seed yields (FIGS. 13B and 13C). This increase in seed yield per plant was due to more seeds per plant with the same size, weight and oil content and composition. Reduction of 20-40% of the CWII protein levels increased seed yield by 150-240%.

### Example 8

### Expression of cwII, GDH, TSR, GLC in Camelina sativa

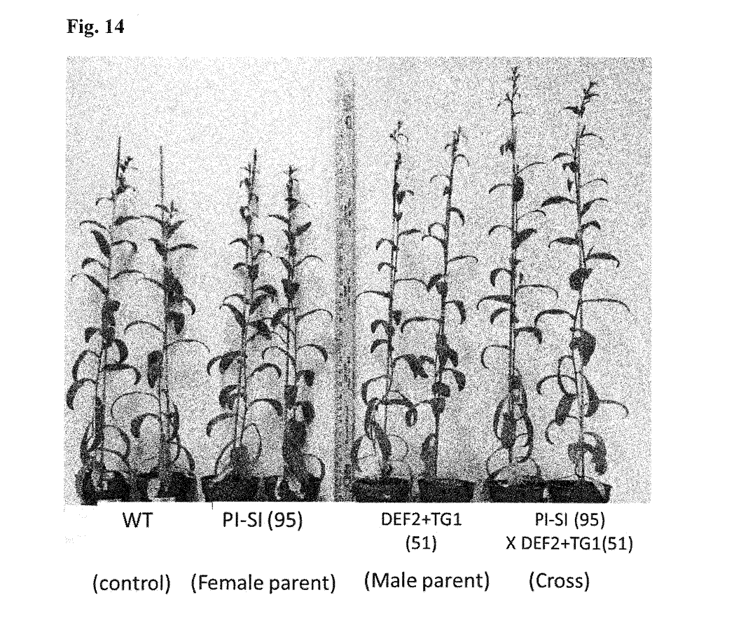

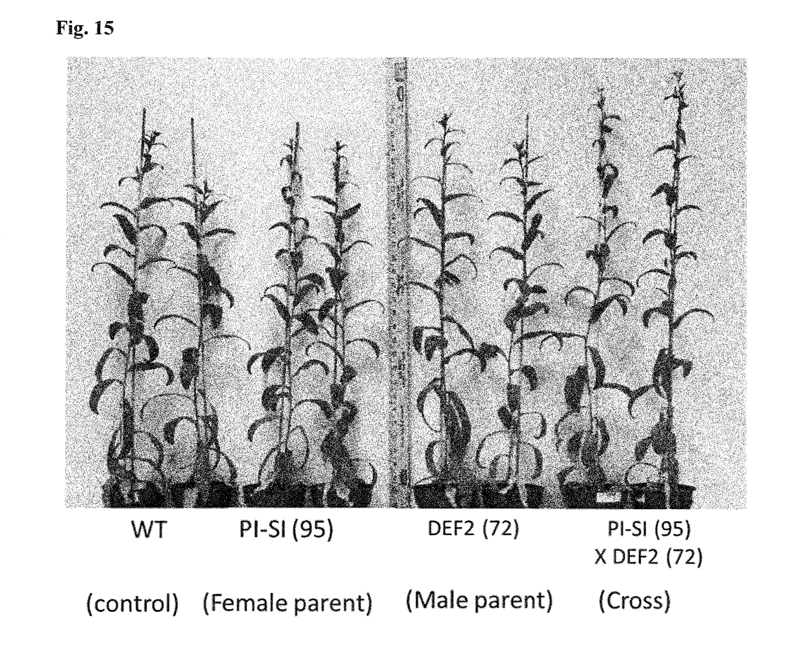

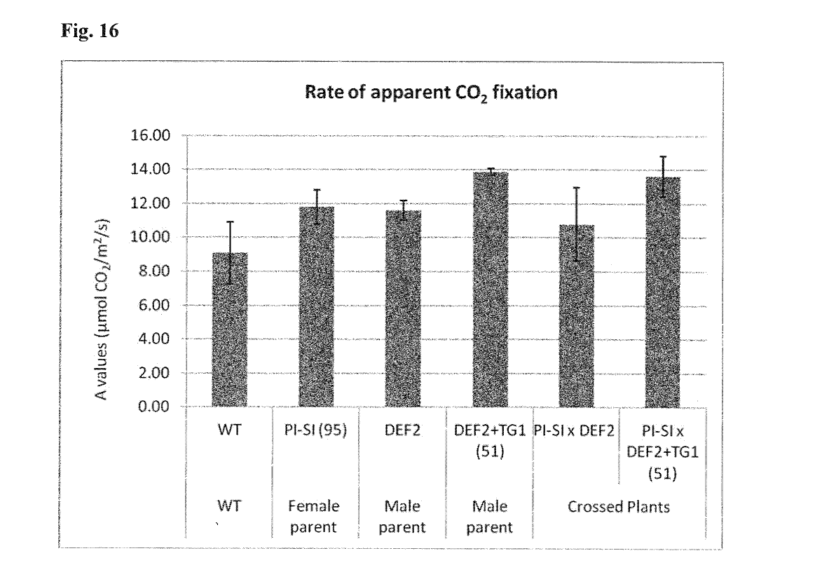

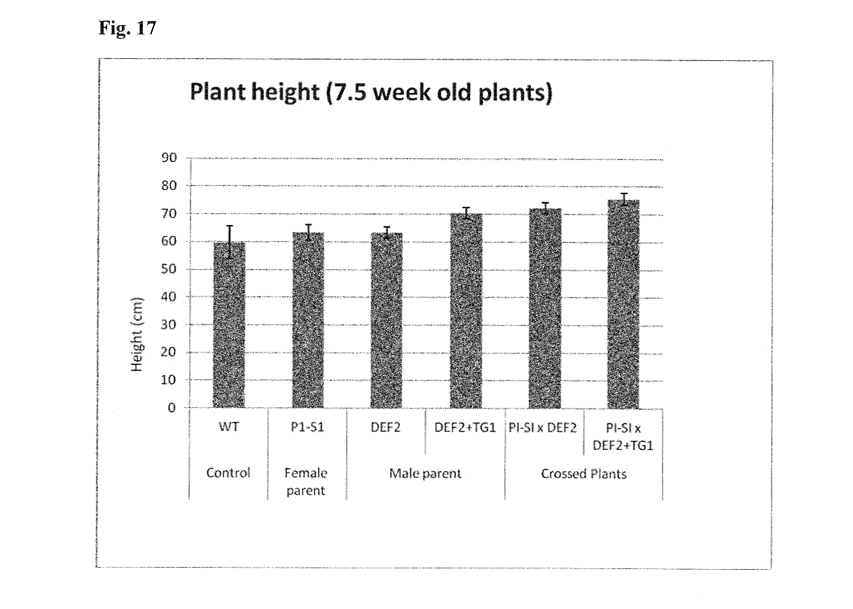

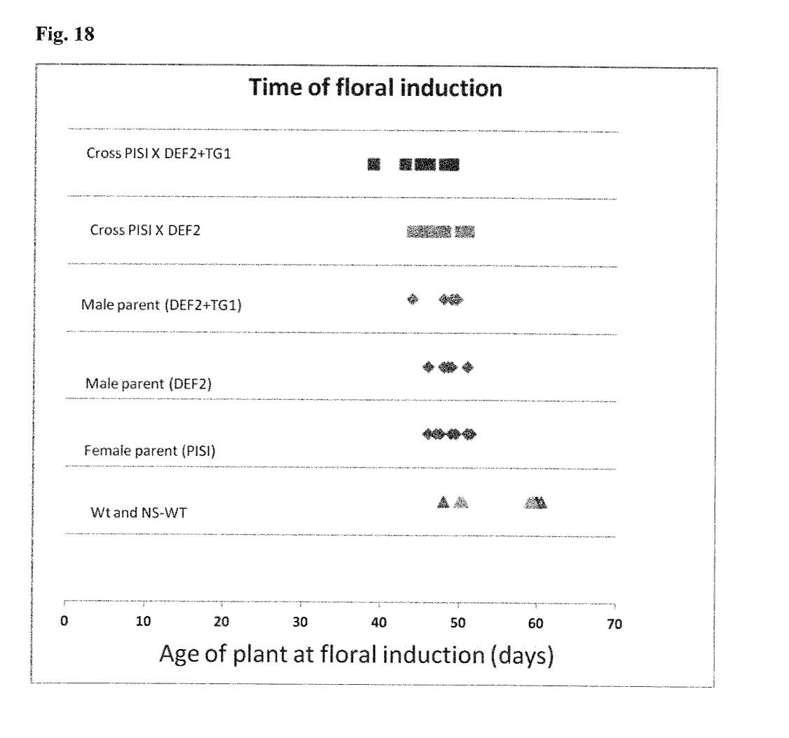

The same methods as used in Examples 1-3 and Example 7 were used to produce plants having a suppressor of cwII and GDH or cwII and GDH, TSR and GCL. Plants were grown under 12 h light/12 h dark) conditions. Light was set at about 50-100 μmol m⁻² s⁻¹ and 20° C. at both day and night time. Relative humidity was 48-50%. The growth phenotypes of plants were monitored daily and photographed weekly. The plants in FIGS. 14 and 15 are seven-weeks-old, and show representative increases in growth of crosses derived from transgenic plants (expressing bypass genes) over WT. For FIG. 16, the rate of photosynthesis was measured using LICOR6400-XT. Four to five week old plants were used for photosynthetic rate determination. Two leaves were selected from each plant and the apparent rate of photosynthesis was measured at 400 ppm CO₂ (ambient CO₂) Rates of photosynthesis (apparent CO₂fixed μmol/m²/s) were compared between leaves of same age from 3-4 independent plants. For FIG. 17, selected plants were photographed from each genotype, and plant heights were measured using the ImageJ software (NIH). For FIG. 18, the flowering times of all plants were monitored daily, and plotted on a graph to visualize the earlier flowering phenotype of the crosses of transgenic plants.

The results above show that introduction of photorespiratory bypass genes (FIG. 1-4) in Camelina plants increases their seed yield productivity by increasing the number of capsules formed per plant. In short-day growth conditions, transgenic plants expressing both full bypass (all five bypass genes) or half bypass (GDH only), show earlier floral induction and an accelerated rate of silique development compared with WT plants. Plants with the silenced cell wall invertase inhibitor show greater plant biomass and higher number of seeds compared to WT plants under long-day conditions. The combination of benefits of both photorespiratory bypass expression and cell wall invertase inhibition can be observed in the crossed plants. CWII-expressing plants crossed with plants expressing GDH alone or GDH, GCL and TSR generate crossed plants which have increased photosynthetic carbon fixation, even earlier floral development, and increased rate of silique formation compared with WT plants and either parents. While in short day growth conditions, the growth benefits of GDH-expressing plants match those in plants expressing GDH, GCL and TSR, in long-day conditions, the latter exceeded the former in growth advantages over WT. In corollary to that, crosses obtained with full bypass and cell wall invertase had growth advantages compared with crosses obtained from half bypass and cell wall invertase.

| | | | | Number of plants |
|---|---|---|---|---|
| | | | | per analysis, p-value |
| | Transgenic | | Increase over | (Student's t-test, |
| Growth feature | genotype | Generation | WT or parents | two tailed) |
| Yield under | DEF2 (72) | T4 | 56.7% over WT | N = 9; |
| short day | | | | p-value = 6.59593E−06 |
| conditions | | | | |
| Yield under | DEF2 + TG1 (51) | T3 | 72.65% over | N = 9 |
| short day | | | WT | p-value = |
| conditions | | | | 2.565E−09 |
| Yield under | aCWII (95) | T3 | 132% over WT | N = 7 (WT N = 6) |
| long day | | | | p = 2.5E−09 |
| conditions | | | | |
| Number of | P1S1 X DEF2 | F1 | 4.2 x WT | 4 < N < 24 |
| siliques | | | 1.25 x aCWII | |
| | | | 0.95 x DEF2 | |
| Number of | P1S1 X | F1 | 7.1 x WT | 4 < N < 24 |
| siliques | DEF2 + TG1 | | 2.1 x aCWII | |
| | | | 2.5 x DEF2 | |

### Example 9

### Modification of the cwII Via the CRISPR-Cas System

An alternative approach to suppressing the cell wall invertase inhibitor is to use genome editing. In the present example, the activity of the cwII is reduced in Camelina using the CRISPR-Cas system.

Camelina (WT or transgenic for GDH or GDH/TSR/GC plants) are transformed (by any method already described for other transgenes) with a nucleotide sequence encoding a CRISPR-associated protein 9 (Cas9) gene under the control of a strong constitutive promoter and at least one single guide RNA (sgRNA) molecule, which comprises a portion of the cwII target gene and which is under a similarly strong constitutive promoter. The Cas9 and the sgRNA can either be on the same construct or in separate construct, to be transformed into plants simultaneously or consecutively (for example, transgenic plants recovered from one transformation can be transformed with the other construct).

Cas9 interacts with a guide RNA molecule to create double-stranded breaks in genomic DNA at the site of homology to the guide RNA (e.g., cwII). Repair is done by the cell through non-homologous end joining and causes indels that shift the reading frame of a coding sequence thus resulting in an inactive target protein. The Cas9 transgene is expressed with a nuclear targeting peptide.

The sgRNA is designed to be 19-22 nt long with full sequence homology to a region of interest within the target gene (e.g., cwII). The target sequence must be followed by a protospacer adjacent motif (PAM) and thus is selected with this in mind. The sgRNA can be used individually or multiplexed to achieve multiple edits within a single gene or multiple genes. The sequence is then queried against any available genomic database to screen for homology that could result in off-target mutations. The optimal sequences are perfect matches to their targets and have no homology in other sites in the genome.

### Example 10

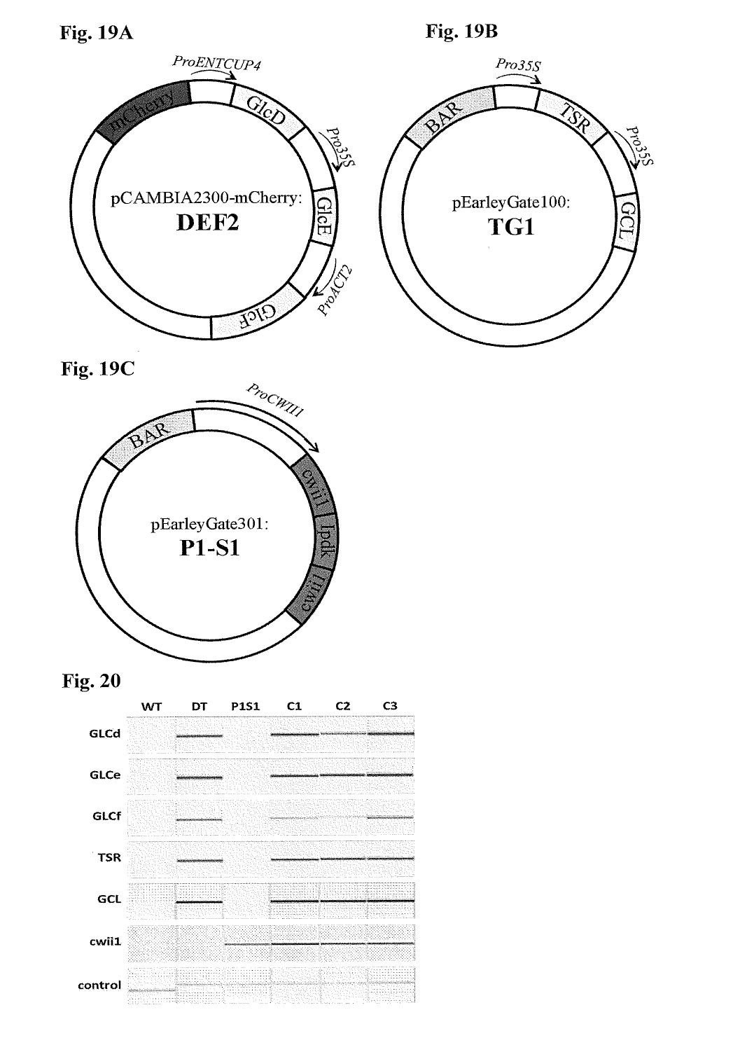

Camelina plants were transformed with the DEF1 and TG1 constructs (FIG. 19A, FIG. 19B) to produce plants expressing the full bypass and with the P1-S1 construct (FIG. 19C) to produce plants expressing the RNAi directed to cell wall invertase inhibitor (cwII) (P1S1). Crosses were made to produce offspring comprising DEF1, TG1 and P1-S1 expressing the full bypass and the RNAi directed to cwII (C1, C2 and C3). DNA analysis (FIG. 20) of the wild type plants as compared to plants expressing the full bypass alone (DT), plants expressing the RNAi directed to cwII (P1S1), and plants expressing both the full bypass and P1S1 (C1, C2, C3) show the presence of the transgenes.

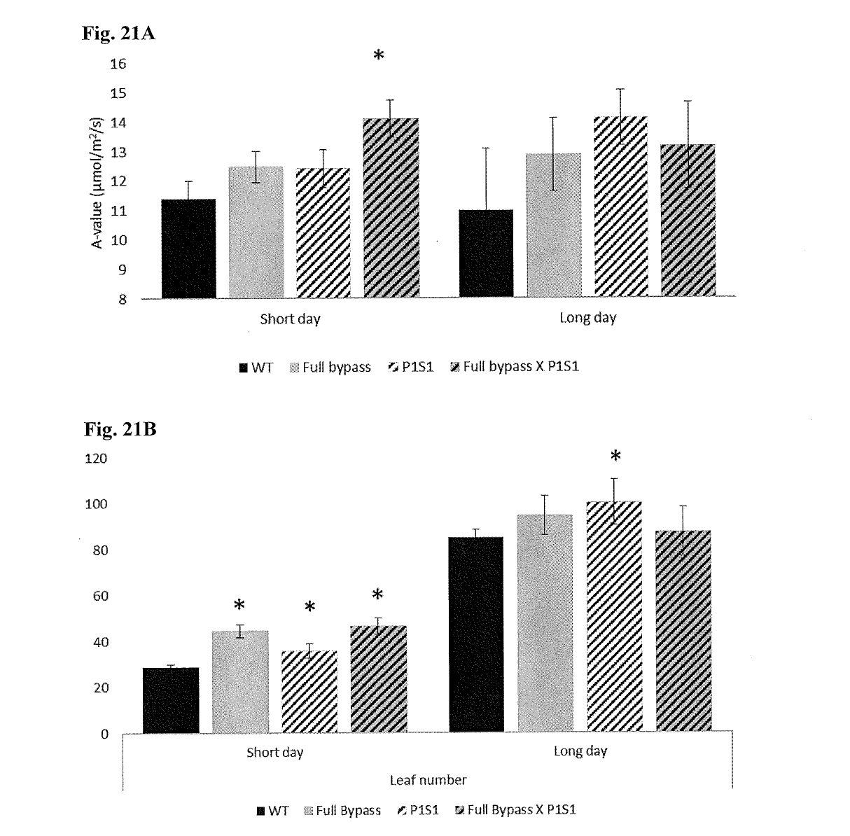

The average photosynthetic rate and leaf number at 10 weeks for WT plants, plants expressing the full bypass, plants expressing P1S1, and plants expressing both the full bypass and P1S1 is shown in FIG. 21A and FIG. 21B, respectively. These results show that the apparent photosynthetic CO2 fixation rate is significantly higher in the Full BypassxP1S1 cross compared to the parent lines and wt under short day conditions, but not under long day conditions. All transgenic lines have significantly more leaves after 10 days of growth under short day conditions. Under long day growth conditions, only the P1S1 parent shows a significant increase in leaf number (P<0.01).

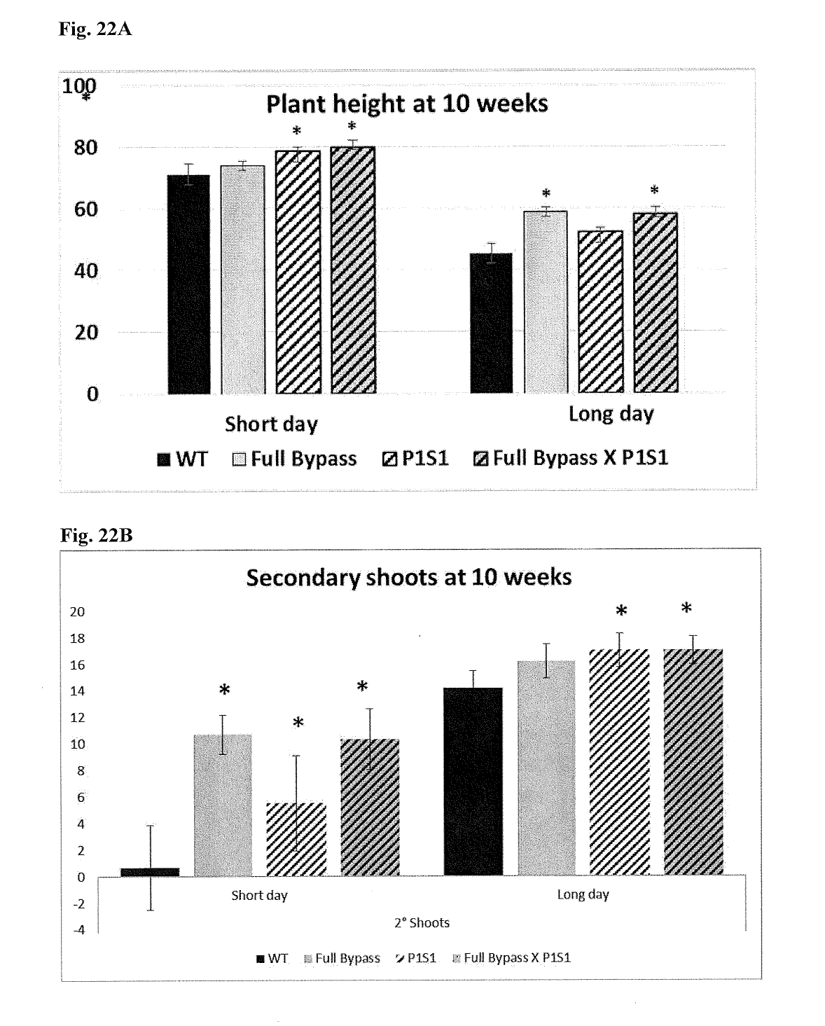

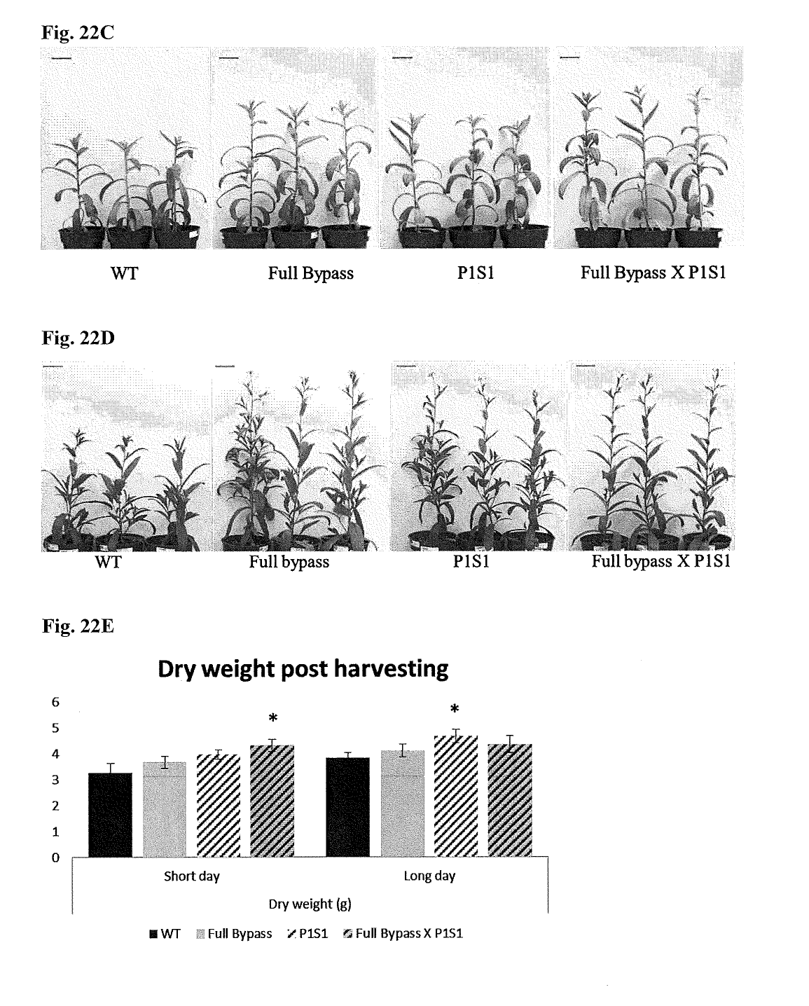

The height at 10 weeks and the number of secondary shoots for WT plants, plants expressing the full bypass, plants expressing P1S1, and plants expressing both the full bypass and P1S1 are shown in FIG. 22A and FIG. 22B, respectively. FIG. 22C and FIG. 22D show the different plants at six weeks of age grown under short and long day conditions and FIG. 22E shows the dry weight of the above ground vegetative biomass post harvest (not including seed) for the different plants. These results show that under short and long day conditions the integrated Full Bypass x P1S1 crosses perform better in height, number of shoots and dry weight of vegetative biomass in short and long day conditions compared to wt. This shows that biomass production is faster when both transgenic pathways—Full Bypass for energy efficient recovery of photorespiratory CO₂ loss and repression of CWII to increase sucrose loading/unloading via the phloem—are present and active in the plants compared to their respective parent lines.

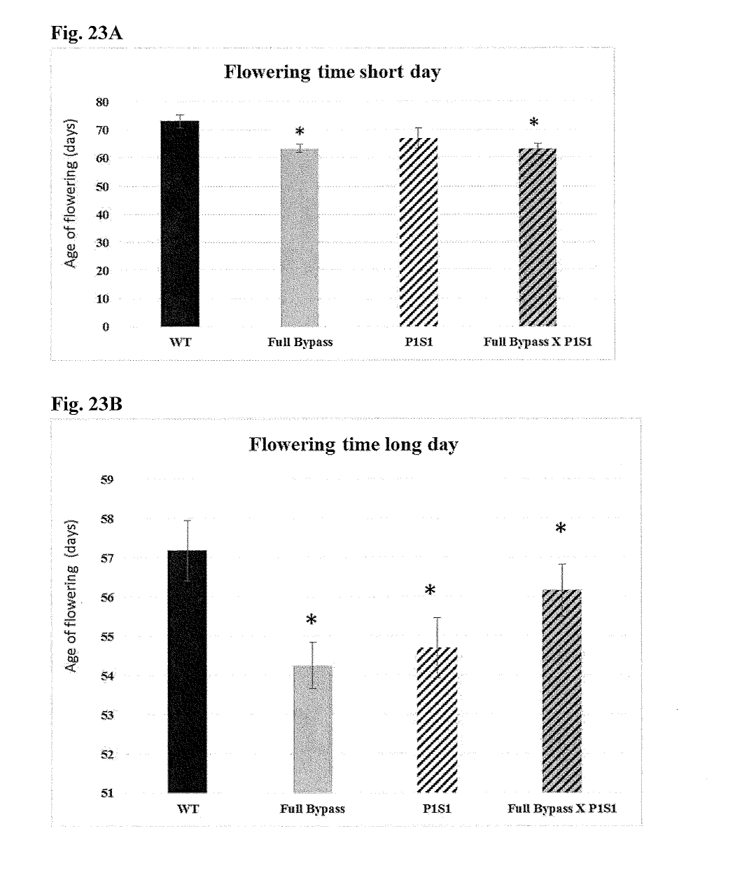

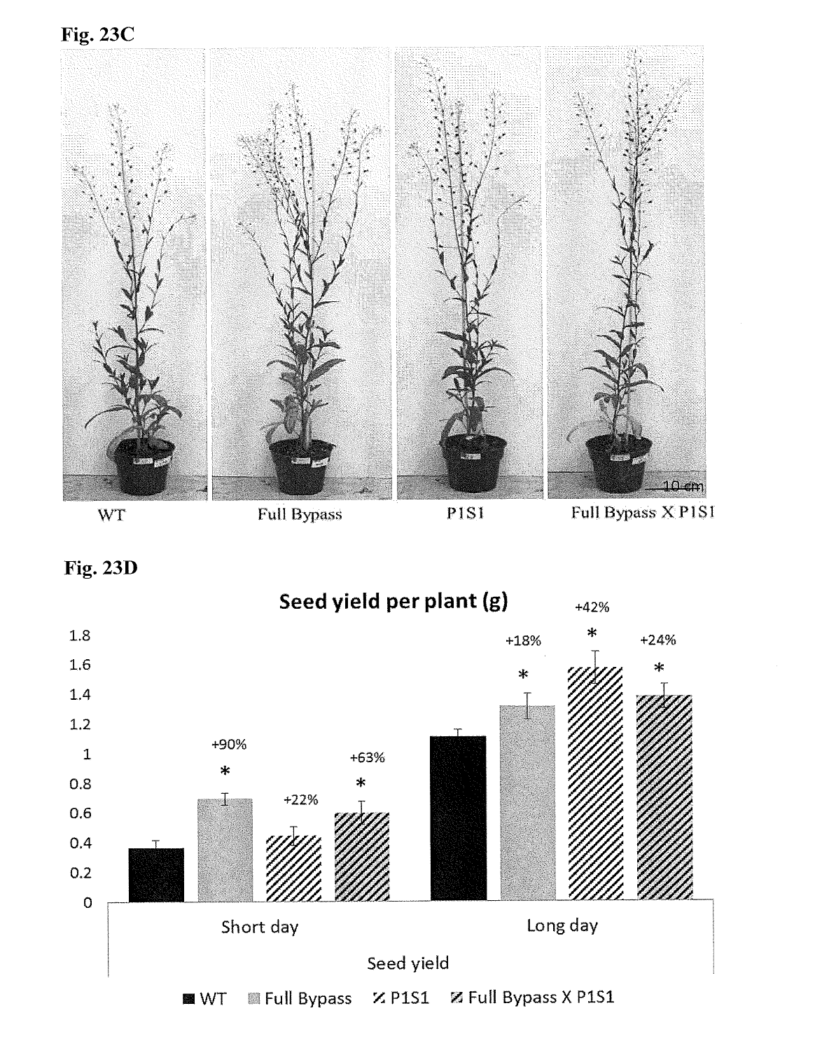

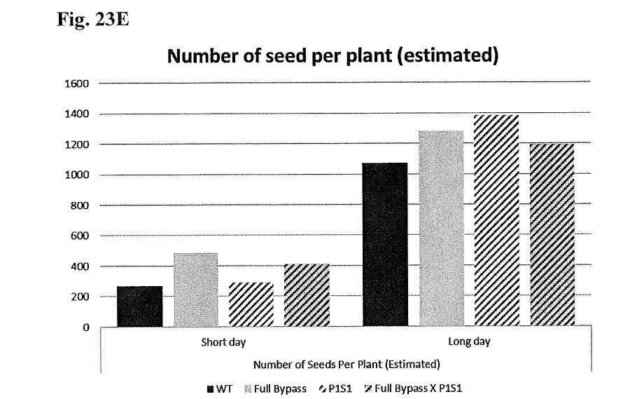

Plant age at the time of flowering for short day and long day grown plants is provided in FIG. 23A and FIG. 23B, respectively, and shows that under short and long day conditions, the integrated Full Bypass x P1S1 lines flowers and matures significantly faster (ca. 1 week) than wt plants. This allows the farmer in the specific geographic region more flexibility for planting and harvesting. Pod production at 10 weeks (long day conditions) for the different plants (FIG. 23C) shows that the transgenic lines have significantly more pods compared to wt plants. Seed yield per plant in grams and the number of seeds per plant (FIGS. 23D and 23E) further support that the integrated Full Bypass line outperforms its parent lines dependent on day length. Under short day conditions, the integrated Full Bypass x P1S1 line had more seeds and higher seed yield (g) than the P1S1 parent, while it outperformed the Full Bypass parent line under long day conditions.

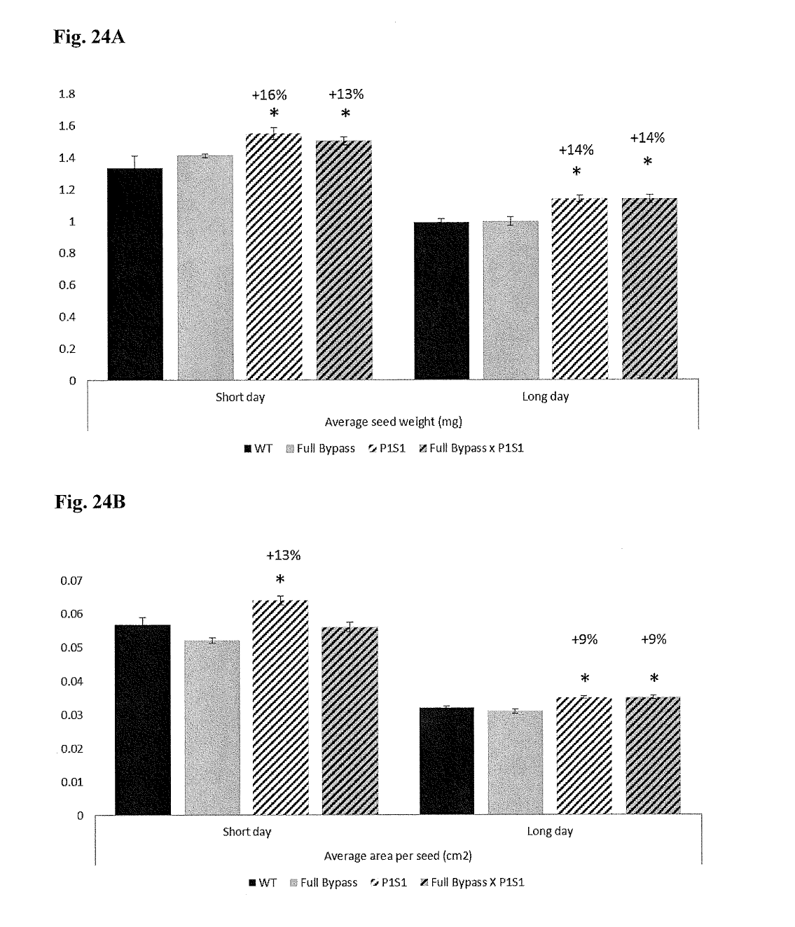

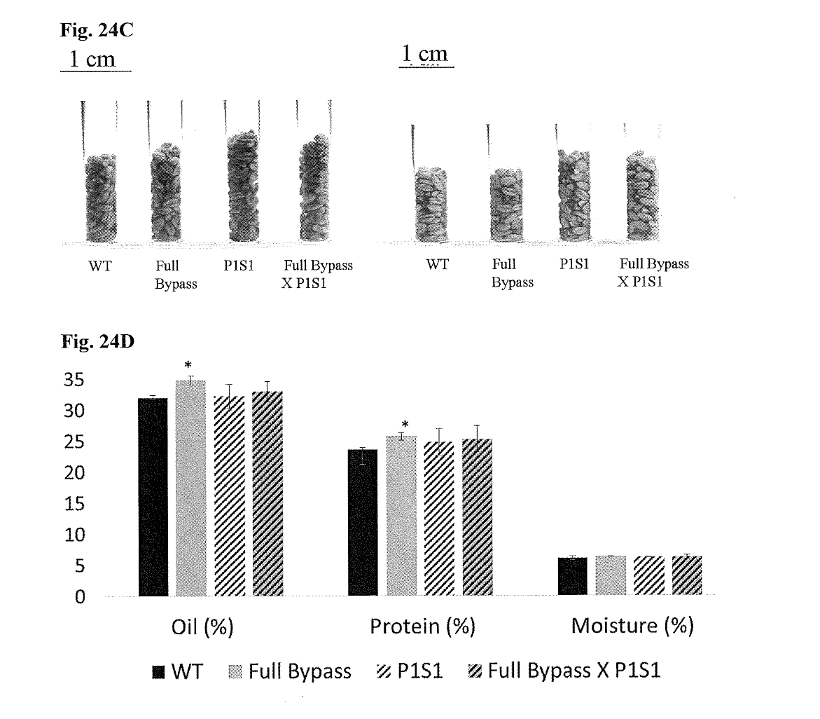

An analysis of the seed produced by each of the different types of plants (e.g., WT, full bypass, P1S1, and full bypass X P1S1) shows that seed yield does not negatively affect seed quality (see, FIGS. 24A-24E).

| | P1S1 | Full Bypass | Crosses | P1S1 | Full Bypass | Crosses |
|---|---|---|---|---|---|---|
| Phenotype | Short day | | | Long day | | |
| (short day) | Percentage increase compared to WT | | | Percentage increase compared to WT | | |
| Increased photosynthesis | 8.98% | 9.44% | 23.71% | 28.37% | 16.99% | 19.86% |
| Increased vegetative | 21.05% | 11.96% | 31.84% | 21.77% | 7.26% | 13.58% |
| biomass (dry weight) | | | | | | |
| Increased seed yield | 21.7% | 89.95% | 63.16% | 41.45% | 18.42% | 24.26% |
| Increased seed weight | 16.23% | 5.75%^{(p>0.05)} | 12.66% | 14.75% | 0.46%^{(p>0.05)} | 14.66% |
| Increased seed area | 12.79% | −8.37%^{(p>0.05)} | −1.37%^{(p>0.05)} | 9.32% | −3.25%^{(p>0.05)} | 9.23% |

The combination of traits in the crosses are important for field growth of the transgenic lines in different climate conditions.

The above examples clearly illustrate the advantages of the invention. Although the present invention has been described with reference to specific details of certain embodiments thereof, it is not intended that such details should be regarded as limitations upon the scope of the invention except as and to the extent that they are included in the accompanying claims.

1-32. (canceled)

33. A method for producing a plant having increased assimilate partitioning directed into fruits and/or seeds and/or other plant part(s) and/or increased seed, root and/or tuber size, comprising:
introducing into a plant cell a heterologous polynucleotide operably linked to a promoter and encoding a suppressor of a cell wall invertase inhibitor (cwII) and/or modifying an endogenous cell wall invertase inhibitor (cwII) gene of a plant cell to produce a plant cell comprising in its genome said modified endogenous cell wall invertase inhibitor gene, wherein said modified endogenous cell wall invertase inhibitor gene encodes a polypeptide having reduced or no cell wall invertase inhibitor activity; and
regenerating a plant from said plant cell, thereby producing a plant having increased assimilate partitioning into fruits and/or seeds and/or other plant part(s) (e.g., root and/or tuber size), and/or increased seed, tuber and/or root size of said plant as compared to a control.

34. The method of claim 33, further comprising:
introducing into the plant cell a heterologous polypeptide operably linked to a promoter and encoding polypeptides having the enzyme activity of glycolate dehydrogenase, a heterologous polynucleotide operably linked to a promoter and encoding a polypeptide having the enzyme activity of tartronic semialdehyde reductase, and a heterologous polynucleotide operably linked to a promoter and encoding a polypeptide having the enzyme activity of glyoxylate carboligase.

35. The method of claim 34, further comprising:
introducing into the plant cell a heterologous polynucleotide operably linked to a promoter and encoding a polypeptide having the activity of a CO₂ transporter.

36. The method of claim 35, wherein the heterologous polynucleotide encoding polypeptides having the enzyme activity of a glycolate dehydrogenase, the heterologous polynucleotide encoding a polypeptide having the activity of a CO₂ transporter, the heterologous polynucleotide encoding a polypeptide having the enzyme activity of a tartronic semialdehyde reductase and/or the heterologous polynucleotide encoding a polypeptide having the enzyme activity of a glyoxylate carboligase are operably linked to a single promoter or to separate promoters, or any combination thereof.

37. The method of claim 35, wherein the heterologous polynucleotide encoding polypeptides having the enzyme activity of a glycolate dehydrogenase, the heterologous polynucleotide encoding a polypeptide having the activity of a CO₂ transporter, the heterologous polynucleotide encoding a polypeptide having the enzyme activity of a tartronic semialdehyde reductase and/or the heterologous polynucleotide encoding a polypeptide having the enzyme activity of a glyoxylate carboligase are introduced into a nucleus and/or a chloroplast of said plant part, and/or plant cell.

38. The method of claim 35, wherein the polypeptides having the enzyme activity of glycolate dehydrogenase, the polypeptide having the enzyme activity of a tartronic semialdehyde reductase and/or the polypeptide having the enzyme activity of a glyoxylate carboligase are each a fusion polypeptide comprising an amino acid sequence that targets said polypeptide to the chloroplast and the polypeptide having the activity of a CO₂ transporter is a fusion polypeptide comprising an amino acid sequence that targets said polypeptide to the membrane.

39. The method of claim 35, wherein the heterologous polynucleotide encoding a polypeptide having the activity of a CO₂ transporter is:
(a) a nucleotide sequence having substantial identity to the nucleotide sequence of SEQ ID NO:14, SEQ ID NO:16, SEQ ID NO:18 or SEQ ID NO:20 or
(b) a nucleotide sequence that encodes an amino acid sequence having substantial identity to an amino acid sequence of SEQ ID NO:15, SEQ ID NO:17, SEQ ID NO:19 or SEQ ID NO:21.

40. The method of claim 33, further comprising introducing into the plant cell an additional heterologous polynucleotide operably linked to a promoter and encoding a polypeptide having the enzyme activity of a cell wall invertase, optionally wherein the additional heterologous polynucleotide operably linked to a promoter and encoding a polypeptide having the enzyme activity of a cell wall invertase is overexpressed.

41. A stably transformed plant, plant part or plant cell, produced by the method of claim 33.

42. A stably transformed plant, plant part or plant cell, produced by the method of claim 34.

43. A stably transformed plant, plant part or plant cell, produced by the method of claim 35.

44. A seed of the stably transformed plant of claim 41, wherein the seed comprises in its genome an endogenous modified cell wall invertase inhibitor gene, wherein said modified gene encodes a polypeptide having reduced or no cell wall invertase inhibitor activity and/or a heterologous polynucleotide operably linked to a promoter and encoding a suppressor of cell wall invertase inhibitor.

45. A seed of the stably transformed plant of claim 42, wherein the seed comprises in its genome (a) an endogenous modified cell wall invertase inhibitor gene, wherein said modified gene encodes a polypeptide having reduced or no cell wall invertase inhibitor activity and/or a heterologous polynucleotide operably linked to a promoter and encoding a suppressor of cell wall invertase inhibitor, (b) a heterologous polynucleotide operably linked to a promoter and encoding polypeptides having the enzyme activity of glycolate dehydrogenase, (c) a heterologous polynucleotide operably linked to a promoter and encoding a polypeptide having the enzyme activity of tartronic semialdehyde reductase, and (d) a heterologous polynucleotide operably linked to a promoter and encoding a polypeptide having the enzyme activity of glyoxylate carboligase.

46. A seed of the stably transformed plant of claim 43, wherein the seed comprises in its genome (a) an endogenous modified cell wall invertase inhibitor gene, wherein said modified gene encodes a polypeptide having reduced or no cell wall invertase inhibitor activity and/or a heterologous polynucleotide operably linked to a promoter and encoding a suppressor of cell wall invertase inhibitor, (b) a heterologous polynucleotide operably linked to a promoter and encoding polypeptides having the enzyme activity of glycolate dehydrogenase, (c) a heterologous polynucleotide operably linked to a promoter and encoding a polypeptide having the enzyme activity of tartronic semialdehyde reductase, (d) a heterologous polynucleotide operably linked to a promoter and encoding a polypeptide having the enzyme activity of glyoxylate carboligase and (e) a heterologous polynucleotide operably linked to a promoter and encoding a polypeptide having the activity of a CO₂ transporter.

47. A crop comprising a plurality of plants according to claim 41, planted together in an agricultural field, a golf course, a residential lawn, a road side, an athletic field, and/or a recreational field.

48. A product produced from the stably transformed plant, plant part or plant cell of claim 41.

49. A product produced from the stably transformed plant, plant part or plant cell of claim 42.

50. The product of claim 48, wherein the product is a food, drink, animal feed, fiber, oil, pharmaceutical, commodity chemical, cosmetic, and/or biofuel.

51. The product of claim 49, wherein the product is a food, drink, animal feed, fiber, oil, pharmaceutical, commodity chemical, cosmetic, and/or biofuel.

52. An expression cassette comprising a heterologous polynucleotide having
(a) the nucleotide sequence of SEQ ID NO:27 and/or SEQ ID NO:28;
(b) a nucleotide sequence that is at least about 80% identical to the nucleotide sequence of (a);
(c) any combination of (a) or (b).

53. The expression cassette of claim 52, wherein the heterologous polynucleotide is operably linked to a polynucleotide of interest.

54. A cell comprising the expression cassette of claim 53.


SEQUENCE LISTING (54 sequences)

SEQ ID NO: 1 - DNA, 1500 bp, Escherichia coli
atgagcatcttgtacgaagagcgtcttgatggcgctttacccgatgtcgaccgcacatcggtactgatggcactgcgtgagcatgtccctggacttgagatcctgcataccgatgaggagatcattccttacgagtgtgacgggttgagcgcgtatcgcacgcgtccattactggttgttctgcctaagcaaatggaacaggtgacagcgattctggctgtctgccatcgcctgcgtgtaccggtggtgacccgtggtgcaggcaccgggctttctggtggcgcgctgccgctggaaaaaggtgtgttgttggtgatggcgcgctttaaagagatcctcgacattaaccccgttggtcgccgcgcgcgcgtgcagccaggcgtgcgtaacctggcgatctcccaggccgttgcaccgcataatctctactacgcaccggacccttcctcacaaatcgcctgttccattggcggcaatgtggctgaaaatgccggcggcgtccactgcctgaaatatggtctgaccgtacataacctgctgaaaattgaagtgcaaacgctggacggcgaggcactgacgctgggatcggacgcgctggattcacctggttttgacctgctggcgctgttcaccggatcggaaggtatgctcggcgtgaccaccgaagtgacggtaaaactgctgccgaagccgcccgtggcgcgggttctgttagccagctttgactcggtagaaaaagccggacttgcggttggtgacatcatcgccaatggcattatccccggcgggctggagatgatggataacctgtcgatccgcgcggcggaagattttattcatgccggttatcccgtcgacgccgaagcgattttgttatgcgagctggacggcgtggagtctgacgtacaggaagactgcgagcgggttaacgacatcttgttgaaagcgggcgcgactgacgtccgtctggcacaggacgaagcagagcgcgtacgtttctgggccggtcgcaaaaatgcgttcccggcggtaggacgtatctccccggattactactgcatggatggcaccatcccgcgtcgcgccctgcctggcgtactggaaggcattgcccgtttatcgcagcaatatgatttacgtgttgccaacgtctttcatgccggagatggcaacatgcacccgttaatccttttcgatgccaacgaacccggtgaatttgcccgcgcggaagagctgggcgggaagatcctcgaactctgcgttgaagttggcggcagcatcagtggcgaacatggcatcgggcgagaaaaaatcaatcaaatgtgcgcccagttcaacagcgatgaaatcacgaccttccatgcggtcaaggcggcgtttgaccccgatggtttgctgaaccctgggaaaaacattcccacgctacaccgctgtgctgaatttggtgccatgcatgtgcatcacggtcatttacctttccctgaactggagcgtttctga

SEQ ID NO: 2 - protein, 499 aa, Escherichia coli
MSILYEERLDGALPDVDRTSVLMALREHVPGLEILHTDEEIIPYECDGLSAYRTRPLLVVLPKQMEQVTAILAVCHRLRVPVVTRGAGTGLSGGALPLEKGVLLVMARFKEILDINPVGRRARVQPGVRNLAISQAVAPHNLYYAPDPSSQIACSIGGNVAENAGGVHCLKYGLTVHNLLKIEVQTLDGEALTLGSDALDSPGFDLLALFTGSEGMLGVTTEVTVKLLPKPPVARVLLASFDSVEKAGLAVGDIIANGIIPGGLEMMDNLSIRAAEDFIHAGYPVDAEAILLCELDGVESDVQEDCERVNDILLKAGATDVRLAQDEAERVRFWAGRKNAFPAVGRISPDYYCMDGTIPRRALPGVLEGIARLSQQYDLRVANVFHAGDGNMHPLILFDANEPGEFARAEELGGKILELCVEVGGSISGEHGIGREKINQMCAQFNSDEITTFHAVKAAFDPDGLLNPGKNIPTLHRCAEFGAMHVHHGHLPFPELERF

SEQ ID NO: 3 - DNA, 1053 bp, Escherichia coli
atgctacgcgagtgtgattacagccaggcgctgctggagcaggtgaatcaggcgattagcgataaaacgccgctggtgattcagggcagcaatagcaaagcctttttaggtcgccctgtcaccgggcaaacgctggatgttcgttgtcatcgcggcattgttaattacgacccgaccgagctggtgataaccgcgcgtgtcggaacgccgctggtgacaattgaagcggcgctggaaagcgcggggcaaatgctcccctgtgagccgccgcattatggtgaagaagccacctggggcgggatggtcgcctgcgggctggcggggccgcgtcgcccgtggagcggttcggtccgcgattttgtcctcggcacgcgcatcattaccggcgctggaaaacatctgcgttttggtggcgaagtgatgaaaaacgttgccggatacgatctctcacggttaatggtcggaagctacggttgtcttggcgtgctcactgaaatctcaatgaaagtgttaccgcgaccgcgcgcctccctgagcctgcgtcgggaaatcagcctgcaagaagccatgagtgaaatcgccgagtggcaactccagccattacccattagtggcttatgttacttcgacaatgcgttgtggatccgccttgagggcggcgaaggatcggtaaaagcagcgcgtgaactgctgggtggcgaagaggttgccggtcagttctggcagcaattgcgtgaacaacaactgccgttcttctcgttaccaggtaccttatggcgcatttcattacccagtgatgcgccgatgatggatttacccggcgagcaactgatcgactggggcggggcgttacgctggctgaaatcgacagccgaggacaatcaaatccatcgcatcgcccgcaacgctggcggtcatgcgacccgctttagtgccggagatggtggctttgccccgctatcggctcctttattccgctatcaccagcagcttaaacagcagctcgacccttgcggcgtgtttaaccccggtcgcatgtacgcggaactttga

SEQ ID NO: 4 - protein, 350 aa, Escherichia coli
MLRECDYSQALLEQVNQAISDKTPLVIQGSNSKAFLGRPVTGQTLDVRCHRGIVNYDPTELVITARVGTPLVTIEAALESAGQMLPCEPPHYGEEATWGGMVACGLAGPRRPWSGSVRDFVLGTRIITGAGKHLRFGGEVMKNVAGYDLSRLMVGSYGCLGVLTEISMKVLPRPRASLSLRREISLQEAMSEIAEWQLQPLPISGLCYFDNALWIRLEGGEGSVKAARELLGGEEVAGQFWQQLREQQLPFFSLPGTLWRISLPSDAPMMDLPGEQLIDWGGALRWLKSTAEDNQIHRIARNAGGHATRFSAGDGGFAPLSAPLFRYHQQLKQQLDPCGVFNPGRMYAEL

SEQ ID NO: 5 - DNA, 1224 bp, Escherichia coli
atgcaaacccaattaactgaagagatgcggcagaacgcgcgcgcgctggaagccgacagcatcctgcgcgcctgtgttcactgcggattttgtaccgcaacctgcccaacctatcagcttctgggcgatgaactggacgggccgcgcgggcgcatctatctgattaaacaggtgctggaaggcaacgaagtcacgcttaaaacacaggagcatctcgatcgctgcctcacttgccgtaattgtgaaaccacctgtccttctggtgtgcgctatcacaatttgctggatatcgggcgtgatattgtcgagcagaaagtgaaacgcccactgccggagcgaatactgcgcgaaggattgcgccaggtagtgccgcgtccggcggtcttccgtgcgctgacgcaggtagggctggtgctgcgaccgtttttaccggaacaggtcagagcaaaactgcctgctgaaacggtgaaagctaaaccgcgtccgccgctgcgccataagcgtcgggttttaatgttggaaggctgcgcccagcctacgctttcgcccaacaccaacgcggcaactgcgcgagtgctggatcgtctggggatcagcgtcatgccagctaacgaagcaggctgttgtggcgcggtggactatcatcttaatgcgcaggagaaagggctggcacgggcgcgcaataatattgatgcctggtggcccgcgattgaagcaggtgccgaggcaattttgcaaaccgccagcggctgcggcgcgtttgtcaaagagtatgggcagatgctgaaaaacgatgcgttatatgccgataaagcgcgtcaggtcagtgaactggcggtcgatttagtcgaacttctgcgcgaggaaccgctggaaaaactggcaattcgcggcgataaaaagctggccttccactgtccgtgtaccctacaacatgcgcaaaagctgaacggcgaagtggaaaaagtgttgcttcgtcttggatttaccttaacggacgttcccgacagccatctgtgctgcggttcagcgggaacatatgcgttaacgcatcccgatctggcacgccagctgcgggataacaaaatgaatgcgctggaaagcggcaaaccggaaatgatcgtcaccgccaacattggttgccagacgcatctggcgagcgccggtcgtacctctgtgcgtcactggattgaaattgtagaacaagcccttgaaaaggaataa

SEQ ID NO: 6 - protein, 407 aa, Escherichia coli
MQTQLTEEMRQNARALEADSILRACVHCGFCTATCPTYQLLGDELDGPRGRIYLIKQVLEGNEVTLKTQEHLDRCLTCRNCETTCPSGVRYHNLLDIGRDIVEQKVKRPLPERILREGLRQVVPRPAVFRALTQVGLVLRPFLPEQVRAKLPAETVKAKPRPPLRHKRRVLMLEGCAQPTLSPNTNAATARVLDRLGISVMPANEAGCCGAVDYHLNAQEKGLARARNNIDAWWPAIEAGAEAILQTASGCGAFVKEYGQMLKNDALYADKARQVSELAVDLVELLREEPLEKLAIRGDKKLAFHCPCTLQHAQKLNGEVEKVLLRLGFTLTDVPDSHLCCGSAGTYALTHPDLARQLRDNKMNALESGKPEMIVTANIGCQTHLASAGRTSVRHWIEIVEQALEKE

SEQ ID NO: 7 - DNA, 1500 bp, Artificial
  Codon optimized GlcD sequence for expression in plants
atgagtattttgtacgaggagagacttgatggagcattgcctgatgtggataggaccagtgttttgatggctttaagagagcatgtgcctggactcgaaattttacataccgatgaagagattatcccatatgagtgtgatggtctcagtgcttacagaacaaggcctcttttggttgtgttgccaaagcagatggaacaggttaccgctatcctcgcagtgtgccacagattaagggttcctgttgtgacaagaggagctggtaccggactttcaggaggtgcactcccattagaaaagggagttctcttagtgatggctagattcaaggagattttggatatcaatcctgtgggaagaagggctagagttcaaccaggtgtgaggaatcttgctattagtcaggctgttgcacctcataacttgtattacgctcctgatccatcttcacaaatagcatgttctattggtggtaatgtggctgagaacgcaggaggtgttcattgccttaagtatggattgacagtgcacaaccttttgaaaattgaagttcagactcttgatggagaggctcttacattgggttctgatgcattggattcacctggttttgatctcttagctttgttcaccggttctgaaggaatgctcggtgttactacagaggttactgtgaagctcttgccaaaacctccagttgctagagtgctcttagcatcatttgatagtgtggaaaaagctggacttgcagttggagatataattgctaatggtatcatacctggaggtcttgaaatgatggataacttgtctattagagccgcagaggatttcatccatgctggatatccagttgatgctgaggcaattcttttgtgtgaacttgatggtgttgagtcagatgtgcaagaagattgcgagagagttaatgatatcctcttaaaggctggagcaactgatgtgaggttggctcaggatgaagcagagagagttaggttttgggctggaagaaaaaacgctttccccgcagttggtaggatatcaccagattattactgtatggatggtacaattcctagaagggctctcccaggagttttagaaggtattgcaagactcagtcaacagtacgatcttagggttgctaatgtgttccatgcaggagatggaaacatgcaccctctcatcttatttgatgctaatgagccaggagagttcgctagagcagaagagcttggaggaaagattcttgaattgtgtgttgaagtgggaggtagtatatctggtgaacatggtatcggaagagagaagattaatcaaatgtgcgctcagtttaactctgatgaaattaccactttccatgctgttaaggctgcattcgatcctgatggacttttgaatcctggaaagaatatcccaactcttcacagatgcgctgagttcggagcaatgcatgttcatcacggtcaccttccttttccagaattggagaggttctga

SEQ ID NO: 8 - DNA, 1053 bp, Artificial
  Codon optimized GlcE sequence for expression in plants
atgctcagagagtgcgattacagtcaggctttattagaacaagtgaaccaggctattagtgataagacaccattagtgatacagggatctaattcaaaggcttttcttggtagacctgtgactggacaaacattggatgttagatgtcataggggtattgtgaactatgatcctactgaattggttatcacagctagagtgggaaccccacttgttactattgaagctgcattggagtctgctggtcagatgctcccatgtgagcctccacactacggagaagaggcaacatggggtggtatggttgcttgcggacttgcaggtcctagaaggccatggagtggttctgttagagattttgtgctcggaacaaggattatcaccggagctggaaagcatcttagattcggaggtgaagttatgaaaaatgtggcaggttatgatcttagtaggttgatggttggatcttacggttgtctcggagtgttaaccgagatctctatgaaggttttgcctagaccaagggcttcactcagtttaagaagggaaatctcactccaagaggctatgagtgaaatagcagagtggcaacttcagcctttgccaataagtggattgtgctattttgataacgctctttggattagattggaaggaggagagggttcagtgaaagctgcaagggaacttttgggaggtgaagaggttgcaggacagttctggcaacagcttagagagcaacagttgcctttcttttctctcccaggtactttatggaggatctctcttccttcagatgctccaatgatggatctccctggagaacaattaatagattggggaggtgctctcagatggttaaagtcaaccgcagaggataatcagatacatagaattgctaggaacgcaggaggtcacgctactagattttcagcaggagatggaggtttcgctcctctttcagcaccattgtttagatatcaccaacagctcaaacaacagttagatccttgcggtgttttcaatccaggaagaatgtacgctgaactttga

SEQ ID NO: 9 - DNA, 1224 bp, Artificial
  Codon optimized GlcF sequence for expression in plants
atgcagacccaacttactgaggagatgaggcaaaacgcaagggctttagaggcagattcaatacttagggcttgtgttcactgtggattttgcaccgctacttgtcctacatatcagcttttgggagatgaattggatggaccaaggggtaggatatacctcattaagcaagttttagagggtaatgaggttactcttaaaacacaggaacatcttgatagatgtttgacctgtaggaattgtgagactacatgccctagtggagttaggtatcacaacctcttagatatcggtagagatatagttgagcagaaggtgaaaagacctttgccagaaagaatactcagggagggattaagacaagttgtgcctaggccagctgtgtttagagcacttacccaagttggtcttgtgttgaggcctttcctcccagaacaggttagagcaaagttaccagcagagactgtgaaggctaaaccaagacctccactcaggcataaaagaagggttctcatgttagaaggatgcgctcagcctactctttcaccaaataccaacgctgcaactgctagagttcttgataggttgggtattagtgtgatgcctgcaaatgaggctggatgttgcggtgctgttgattaccatcttaacgcacaagagaagggattggctagagcaaggaataacatagatgcttggtggccagcaattgaagctggtgcagaggctatcttgcaaactgcttctggatgtggtgcattcgttaaggaatatggacagatgcttaaaaatgatgcattgtacgctgataaggcaagacaagtgtcagaacttgctgttgatttggtggagcttttgagagaagagcctctcgagaaattagctatcagaggagataagaaacttgcatttcattgtccatgcacactccaacacgctcagaagttaaacggagaagttgagaaagtgctcttaagactcggtttcacattaaccgatgttcctgatagtcatctttgttgcggatctgctggtacatatgcattgacccacccagatcttgctagacaattgagggataataagatgaacgcattggaatcaggaaaaccagagatgattgttactgctaacatcggatgtcagacacatctcgcatctgctggtagaacatcagttaggcactggattgaaatcgtggagcaagctcttgaaaaagagtga

SEQ ID NO: 10 - DNA, 1782 bp, Artificial
  Codon optimized gloxylate carboligase coding sequence for expression in plants
atggctaagatgagggctgtggatgctgctatgtatgtgcttgaaaaggagggaataactaccgcatttggtgtgcctggtgctgctattaatcctttctattcagctatgagaaagcatggaggtatcagacacatattggcaaggcatgtggaaggtgctagtcatatggcagagggatacaccagagctactgctggaaacattggagtttgtcttggtactagtggaccagctggtacagatatgatcaccgcactctatagtgcttctgctgattctattcctatcttatgcatcacaggtcaagctccaagagcaaggcttcacaaagaagatttccaggctgtggatattgaggctatcgcaaagcctgtttctaaaatggctgtgactgttagagaagctgcacttgtgccaagggttttgcaacaggcttttcatttgatgagatcaggaaggcctggtccagtgctcgttgatcttcctttcgatgtgcaagttgctgaaattgagtttgatcctgatatgtatgaacctcttccagtgtacaagccagctgcatctagaatgcaaatcgaaaaagctgttgagatgttgattcaggcagagaggcctgtgatcgttgctggaggtggagttattaatgcagatgctgctgctcttttgcaacagtttgctgaactcacctcagtgcctgttatcccaactttaatgggttggggatgtattcctgatgatcacgagctcatggctggaatggtgggtttacaaactgcacatagatacggtaacgctacactcttagcatctgatatggttttcggtattggaaatagatttgctaacaggcacacaggttcagtggaaaagtacactgagggaagaaaaattgttcatattgatattgagcctacccagatcggtagggtgctttgcccagatttgggaatagtttctgatgctaaggcagctttaacacttttggtggaagttgctcaagagatgcagaaggcaggaagactcccatgtaggaaagaatgggttgctgagtgccaacagagaaagaggactctcctcagaaaaacacatttcgataacgtgcctgttaagccacaaagagtttatgaagagatgaacaaagcttttggtagggatgtgtgttacgttactacaatcggactttctcaaatagcagctgcacagatgttgcacgttttcaaagatagacattggataaactgtggacaggctggtcctcttggatggactatcccagctgcattgggtgtttgcgctgctgatcctaagagaaacgttgtggctataagtggagatttcgatttccaattcctcatcgaagagttagctgttggagcacagtttaaaataccatacattcacgtgttggttaataacgcttaccttggattgattagacaatcacagagggctttcgatatggattactgtgttcaacttgcattcgaaaatatcaactcttcagaagtgaatggttacggagttgatcatgtgaaggttgctgaaggtctcggatgcaaggcaataagagttttcaaacctgaagatattgctccagcatttgagcaagctaaagcacttatggctcagtacagagttcctgttgtggttgaagtgattttggagagggttacaaatatctcaatgggaagtgagctcgataacgttatggaattcgaggatattgctgataacgctgctgatgctccaactgagacttgttttatgcactacgaatga

SEQ ID NO: 11 - protein, 593 aa, Escherichia coli
MAKMRAVDAAMYVLEKEGITTAFGVPGAAINPFYSAMRKHGGIRHILARHVEGASHMAEGYTRATAGNIGVCLGTSGPAGTDMITALYSASADSIPILCITGQAPRARLHKEDFQAVDIEAIAKPVSKMAVTVREAALVPRVLQQAFHLMRSGRPGPVLVDLPFDVQVAEIEFDPDMYEPLPVYKPAASRMQIEKAVEMLIQAERPVIVAGGGVINADAAALLQQFAELTSVPVIPTLMGWGCIPDDHELMAGMVGLQTAHRYGNATLLASDMVFGIGNRFANRHTGSVEKYTEGRKIVHIDIEPTQIGRVLCPDLGIVSDAKAALTLLVEVAQEMQKAGRLPCRKEWVAECQQRKRTLLRKTHFDNVPVKPQRVYEEMNKAFGRDVCYVTTIGLSQIAAAQMLHVFKDRHWINCGQAGPLGWTIPAALGVCAADPKRNVVAISGDFDFQFLIEELAVGAQFKIPYIHVLVNNAYLGLIRQSQRAFDMDYCVQLAFENINSSEVNGYGVDHVKVAEGLGCKAIRVFKPEDIAPAFEQAKALMAQYRVPVVVEVILERVTNISMGSELDNVMEFEDIADNAADAPTETCFMHYE

SEQ ID NO: 12 - DNA, 879 bp, Artificial
  Codon optimized tartronic semialdehyde reductase coding sequence for expression in plants
atgaagttaggttttatcggtctcggtattatgggaacaccaatggcaatcaatctcgcaagggctggacaccaattacacgttacagctattggacctgttgcagatgaacttttgtcacttggtgctgttagtgtggaaaccgcaagacaagttactgaggcttctgatataatctttattatggtgcctgatactccacaggttgaagaggtgctcttcggagagaatggttgtacaaaggcttcattaaagggaaaaaccatcgttgatatgtcttcaatcagtcctatagaaaccaaaagatttgctagacaagttaacgagcttggaggagattatttggatgcaccagtgagtggaggtgaaattggagctagagagggtactctttctatcatggttggaggagatgaagctgtttttgagagggtgaagcctctcttcgaactcctcggaaaaaatatcactctcgtgggtggtaacggagatggtcaaacatgcaaggttgcaaatcagataattgtggctttgaacatagaagcagtttctgaggctcttttgtttgcatcaaaagctggtgcagatccagttagagtgaggcaggcacttatgggaggtttcgctagttctagaatattggaagttcatggagagagaatgataaagagaacttttaatcctggattcaagatcgcactccaccaaaaagatctcaacttagctcttcagtctgctaaagcattggctctcaatcttccaaacactgctacatgtcaagagttgttcaatacctgcgctgcaaacggaggttcacagttggatcacagtgctctcgtgcaggctttagaactcatggcaaaccacaaactcgcataa

SEQ ID NO: 13 - protein, 292 aa, Escherichia coli
MKLGFIGLGIMGTPMAINLARAGHQLHVTAIGPVADELLSLGAVSVETARQVTEASDIIFIMVPDTPQVEEVLFGENGCTKASLKGKTIVDMSSISPIETKRFARQVNELGGDYLDAPVSGGEIGAREGTLSIMVGGDEAVFERVKPLFELLGKNITLVGGNGDGQTCKVANQIIVALNIEAVSEALLFASKAGADPVRVRQALMGGFASSRILEVHGERMIKRTFNPGFKIALHQKDLNLALQSAKALALNLPNTATCQELFNTCAANGGSQLDHSALVQALELMANHKLA

SEQ ID NO: 14 - DNA, 861 bp, Camelina sativa
atggcagaaaacaaagaagaagatgttaagcttggagctaacaaattcagagaaacacagccattaggaacagctgctcaaacagacaaagattacaaagaaccaccaccagctcctttgtttgaaccaggggaattatcatcatggtcattttacagagctggaattgcagaatttatggctactttcttgtttttgtacatcactatcttgactgttatgggtcttaagagatctgatagtctgtgtagttcagttggtattcaaggtgttgcttgggcttttggtggtatgatctttgctttggtttactgtactgctggtatctcaggaggacacatcaacccagctgtgacctttggattgttcttggcaaggaaactgtccttaaccagggctattttctacatagtgatgcaatgccttggtgcaatttgtggtgctggtgttgtgaagggattcatggttggtccataccagagacttggtggtggtgctaatgttgttaaccatggttacaccaaaggtgatggccttggtgctgaaattattggcacttttgtccttgtttacactgttttctctgctactgatgctaagagaaatgccagagactcacatgttcctattttggcaccacttcccatcggattcgcggttttcttggttcatttggccaccattcccatcaccggaactggcatcaaccccgctaggagtcttggagctgcgatcatctacaacacagaccaggcatgggacgaccactggatcttttgggttggaccattcattggagctgcacttgctgcagtttaccatcaaataatcatcagagccattccattccacaagtcgtct

SEQ ID NO: 15 - protein, 287 aa, Camelina sativa
MAENKEEDVKLGANKFRETQPLGTAAQTDKDYKEPPPAPLFEPGELSSWSFYRAGIAEFMATFLFLYITILTVMGLKRSDSLCSSVGIQGVAWAFGGMIFALVYCTAGISGGHINPAVTFGLFLARKLSLTRAIFYIVMQCLGAICGAGVVKGFMVGPYQRLGGGANVVNHGYTKGDGLGAEIIGTFVLVYTVFSATDAKRNARDSHVPILAPLPIGFAVFLVHLATIPITGTGINPARSLGAAIIYNTDQAWDDHWIFWVGPFIGAALAAVYHQIIIRAIPFHKSS

SEQ ID NO: 16 - DNA, 2496 bp, Synechococcus sp. PCC 7002
ccgtaagcatcaacgattctttacatcatcatccatcggcgcgacttgctcacatcgcagcattaagattgcagttgccatagccacaatcccagaaaaaattcacgatccagtacccgaaagcctttttttaaaccaattttagataagttttagttatttttttatccaaaaagacttaagtccagcttatttacatgtcatggccttaggactatattaaatctcacatccatagtcgaaagactatcaacaggccaagtttaagggcaatgtccttgaggattctgccctttctctcagtttttcatcattgattcttcgatcaattgagtacagcacctagttaaagcaaacacaaatatatgaatcaatacagtcatcgtaaatttttgatcaccactggcgtggcagcgggcagcttatccatattttctttgtagtaattagagttttagcacagaaacaattggaactttcttgggcattttaaacaattttatatttatcgaggaggaatctactgttatgagacaacagcaacttttttggctgactactttgatcgttgggggcaatatttttcaggctgctacgccactacaggcccaggaaattaatttgacaacatcgctgagttcaccaacactacaggattctcgctatctagcctcggcctccatgggacaaatggcctcagtatctagattacgggacgtgaagccgacggattgggcttatgaagcactacaaagtctggtggaacggtatggttgcattgttggttatccagatcaaacattccgcggcgatcgccccctgagccgttatgaatttgccgccggactaaatgcttgcctcaatgccctagaacggcagatccaaggcaataatgccgatgtatcctccagcgatcttgcaaccctccggcgattgaccaacgagtttcaggcggaattagccaccctcggcacaagggttgatgatctcgaagcccgcaccagtgaactcgaaaaccaacaattttcaacgaccacaaaactgaatggagaagctattttctctatcagtggggcaacgggtggtgaaccagagggcaacgatgctcagattaccttcaataatcgtctgcggctgaatttgaccaccagttttaccggaaaagatgccctgattactggcttacaagcctacaatttttcggcgggtaaatctattacaggtacaggtaacgttgccgaaactctctttcccaatgatgcctctatccttggggatagcatgactaacctcgcctgggaaccacaatttgctggtttgaatccacaaaatctacaacctagttgcggtaacaatagcctttgtctgtacaagttgctctatgttagaccgatcacagataaattaacggcatttattggcccgaaggcggaagttaccgatgcctttccggcgattcttccctttgctagtgaaggccagggagcactttctcgctttgcaactttgaatccagtattgcggatgtctgggggaaccagtggtacaggactcgcttccgcagctggctttatctataaacccaatgatgtcatcgattggcgggcactctatgggtcagtgaatgcggcaatccctggtaatgaaggttttccggggacgccgttgggggctggcttgttcaatggcagttttatcgccgcaacacaattgacgcttcatcctaatgacaagcttgatctaggtctgaactatgcctacagctaccaccagatcaatattgcgggtacgggtttaacaggagctgagacgcgtattcttggcgatctaccactgaccaccccagtacgatttaactcctttggggcaacagtaaactggcgcgtcagtccaaaagttaacctgacaggttatggggcatacatcatgacagatcaagcgaatagtggctctgcctatacaaatctaagcagttggatggcgggtctgtattttccagatgcattcgcgaagggcaatgcggcagggattttgtttggtcaaccactttatcgggtagatgcgggtaatggggcgagtttaagtccagcaaacattggcgatcgccaaaccccctaccaactggaagccttttatcgccatcaaatcaatgatcacatcagcattacgccgggggcatttgtgattttcaatccagaaggagatgcccaaaatgaaacaaccagcgtttttgcgttgcgtacgacttataccttctagaactaactgatcaccattttacttagtagaaacttatgagtgtttttgttgcggctgatagtattgataaagtatttccgttgtcgggggtggtgaatatattaccctttaatattttttaccttcataaatcatgttcaaaactttaatcaaaaatagtgcggcgatcgcgtttgtacttttaggttccatagccgttattcctggggcaagttcccaaattagtgctactccctt

SEQ ID NO: 17 - protein, 576 aa, Synechococcus sp. PCC 7002
MRQQQLFWLTTLIVGGNIFQAATPLQAQEINLTTSLSSPTLQDSRYLASASMGQMASVSRLRDVKPTDWAYEALQSLVERYGCIVGYPDQTFRGDRPLSRYEFAAGLNACLNALERQIQGNNADVSSSDLATLRRLTNEFQAELATLGTRVDDLEARTSELENQQFSTTTKLNGEAIFSISGATGGEPEGNDAQITFNNRLRLNLTTSFTGKDALITGLQAYNFSAGKSITGTGNVAETLFPNDASILGDSMTNLAWEPQFAGLNPQNLQPSCGNNSLCLYKLLYVRPITDKLTAFIGPKAEVTDAFPAILPFASEGQGALSRFATLNPVLRMSGGTSGTGLASAAGFIYKPNDVIDWRALYGSVNAAIPGNEGFPGTPLGAGLFNGSFIAATQLTLHPNDKLDLGLNYAYSYHQINIAGTGLTGAETRILGDLPLTTPVRFNSFGATVNWRVSPKVNLTGYGAYIMTDQANSGSAYTNLSSWMAGLYFPDAFAKGNAAGILFGQPLYRVDAGNGASLSPANIGDRQTPYQLEAFYRHQINDHISITPGAFVIFNPEGDAQNETTSVFALRTTYTF

SEQ ID NO: 18 - DNA, 948 bp, Thioalkalivibrio sp. K90mix
atggcttttgatccggtagttctgttcttcctgctcggggcgattgccgggctggccaagtcggacctcaagatcccgatggcgatctacgaggcactgtcgatttacctcctgctggccatcggcttgcatggtggcgtgaagctggcggaaagcgagctggtgccgctcatcctgcctggccttgcggtgctgatggtcggggccctgatcccgctgctggcgttcccggtgctgcgctggctggggcatatgccgcgcgcggattcggcctccatcgccgcgcactacgggtcggtcagtgtggtgacgttctcggtggcggtggcctttctcgcggcccgagggatcgactacgagggccacatggtggtcttcctggtgctgctggagatgccggcactggtgatcggcatcctgctggcgcgcatgggcacgaagggaccggtgcaatggggcaagaccatgcacgaggtctttttcggcaagagcatcttcctgctcgccggtgggctggtgatcggattcgtggccggtcccgaactgatggacccactggagccgatgttcttcgatctgttcaagggcgtgctggccctgttcctgctggagatggggctggtcgcctcgagccggatcgccgaggtgcgccagtacgggctgttcctggtagtgttcgcgatcgtgatgccggtggtctcggcgatcctcgggatcctgctgggctggggcctgggcatgagcctgggcggtacgctgctgctggctaccctgtacgcgagtgcgtcctacatcgccgcacccgcggccatgcggatcgcggtccccaaggccaaccccgcgctgtcgatcggggcctcgctgggggttaccttcccgttcaatattttcctgggcgtcccgctgtatttctggatgacccagtggctctactcgttgggaggctag

SEQ ID NO: 19 - protein, 315 aa, Thioalkalivibrio sp. K90mix
MAFDPVVLFFLLGAIAGLAKSDLKIPMAIYEALSIYLLLAIGLHGGVKLAESELVPLILPGLAVLMVGALIPLLAFPVLRWLGHMPRADSASIAAHYGSVSVVTFSVAVAFLAARGIDYEGHMVVFLVLLEMPALVIGILLARMGTKGPVQWGKTMHEVFFGKSIFLLAGGLVIGFVAGPELMDPLEPMFFDLFKGVLALFLLEMGLVASSRIAEVRQYGLFLVVFAIVMPVVSAILGILLGWGLGMSLGGTLLLATLYASASYIAAPAAMRIAVPKANPALSIGASLGVTFPFNIFLGVPLYFWMTQWLYSLGG

SEQ ID NO: 20 - DNA, 864 bp, Nicotiana tabacum
atggcagaaaacaaagaagaagatgttaagcttggagctaacaaattcagagaaacacagccattaggaacagctgctcaaacagacaaagattacaaagaaccaccaccagctcctttgtttgaaccaggggaattatcatcatggtcattttacagagctggaattgcagaatttatggctactttcttgtttttgtacatcactatcttgactgttatgggtcttaagagatctgatagtctgtgtagttcagttggtattcaaggtgttgcttgggcttttggtggtatgatctttgctttggtttactgtactgctggtatctcaggaggacacatcaacccagctgtgacctttggattgttcttggcaaggaaactgtccttaaccagggctattttctacatagtgatgcaatgccttggtgcaatttgtggtgctggtgttgtgaagggattcatggttggtccataccagagacttggtggtggtgctaatgttgttaaccatggttacaccaaaggtgatggccttggtgctgaaattattggcacttttgtccttgtttacactgttttctctgctactgatgctaagagaaatgccagagactcatatgttcctattttggcaccacttcccatcggattcgcggttttcttggttcatttggccaccattcccatcaccggaactggcatcaaccccgctaggagtcttggagctgcgatcatctacaacacagaccaggcatgggacgaccactggatcttttgggttggaccattcattggagctgcacttgctgcagtttaccatcaaataatcatcagagccattccattccacaagtcgtcttaa

SEQ ID NO: 21 - protein, 287 aa, Nicotiana tabacum
MAENKEEDVKLGANKFRETQPLGTAAQTDKDYKEPPPAPLFEPGELSSWSFYRAGIAEFMATFLFLYITILTVMGLKRSDSLCSSVGIQGVAWAFGGMIFALVYCTAGISGGHINPAVTFGLFLARKLSLTRAIFYIVMQCLGAICGAGVVKGFMVGPYQRLGGGANVVNHGYTKGDGLGAEIIGTFVLVYTVFSATDAKRNARDSYVPILAPLPIGFAVFLVHLATIPITGTGINPARSLGAAIIYNTDQAWDDHWIFWVGPFIGAALAAVYHQIIIRAIPFHKSS

SEQ ID NO: 22 - DNA, 1399 bp, Artificial
  cwii1 RNAi sequence
tgcaccagacttcaaactttgtgtctctctactcaactccgacccacgtggctcctctgccgacatctctggcctcgctctcatcctcatcgataaaatcaaggtgctggcgacaaagaccttaaccgagatcaacggtctatataaaaagagaccggaactaaaacaggctttggaccaatgtagtcgaagatacaaaacgattttaaatgctgatgttcccgaagccatcgaagctatctctaaaggagtccctaaattcggcgaagacggcgtgattgacgccggggtagaagcttctgtttgtgcccgggaggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagctcgagcacaaacagaagcttctaccccggcgtcaatcacgccgtcttcgccgaatttagggactcctttagagatagcttcgatggcttcgggaacatcagcatttaaaatcgttttgtatcttcgactacattggtccaaagcctgttttagttccggtctctttttatatagaccgttgatctcggttaaggtctttgtcgccagcaccttgattttatcgatgaggatgagagcgaggccagagatgtcggcagaggagccacgtgggtcggagttgagtagagagacacaaagtttgaagtctggtgcatt

SEQ ID NO: 23 - DNA, 1398 bp, Artificial
  cwii2 RNAi sequence
gtaccatgccaacgcgacaataatcgaatcaacttgcaaaaccacgaacaactacaaattctgtgtctcggctctcaaatccgacccaagaagtcccacagccgacacaaaaggtctcgcagccattatgatcggcgttggtatgacaaacgccacttccaccgcaacttacatcgccggaaacctaacatccgctgcaaacgacgtcgtccttaaaaaggtgttacaagattgctccgagaagtatgctctcgccgctgattctctccgtcaaacaattcaagatcttgatgatgaagcttatgactatgccccgggaggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagctcgaggcatagtcataagcttcatcatcaagatcttgaattgtttgacggagagaatcagcggcgagagcatacttctcggagcaatcttgtaacacctttttaaggacgacgtcgtttgcagcggatgttaggtttccggcgatgtaagttgcggtggaagtggcgtttgtcataccaacgccgatcataatggctgcgagaccttttgtgtcggctgtgggacttcttgggtcggatttgagagccgagacacagaatttgtagttgttcgtggttttgcaagttgattcgattattgtcgcgttggc

SEQ ID NO: 24 - DNA, 2022 bp, Artificial
  cwii1-cwii2 RNAi sequence
tgcaccagacttcaaactttgtgtctctctactcaactccgacccacgtggctcctctgccgacatctctggcctcgctctcatcctcatcgataaaatcaaggtgctggcgacaaagaccttaaccgagatcaacggtctatataaaaagagaccggaactaaaacaggctttggaccaatgtagtcgaagatacaaaacgattttaaatgctgatgttcccgaagccatcgaagctatctctaaaggagtccctaaattcggcgaagacggcgtgattgacgccggggtagaagcttctgtttgtgtctagaccaacgcgacaataatcgaatcaacttgcaaaaccacgaacaactacaaattctgtgtctcggctctcaaatccgacccaagaagtcccacagccgacacaaaaggtctcgcagccattatgatcggcgttggtatgacaaacgccacttccaccgcaacttacatcgccggaaacctaacatccgctgcaaacgacgtcgtccttaaaaaggtgttacaagattgctccgagaagtatgctctcgccgctgattctctccgtcaaacaattcaagatcttgatgatgaagcttatgactatgccccgggaggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagctcgaggcatagtcataagcttcatcatcaagatcttgaattgtttgacggagagaatcagcggcgagagcatacttctcggagcaatcttgtaacacctttttaaggacgacgtcgtttgcagcggatgttaggtttccggcgatgtaagttgcggtggaagtggcgtttgtcataccaacgccgatcataatggctgcgagaccttttgtgtcggctgtgggacttcttgggtcggatttgagagccgagacacagaatttgtagttgttcgtggttttgcaagttgattcgattattgtcgcgttgggctagccacaaacagaagcttctaccccggcgtcaatcacgccgtcttcgccgaatttagggactcctttagagatagcttcgatggcttcgggaacatcagcatttaaaatcgttttgtatcttcgactacattggtccaaagcctgttttagttccggtctctttttatatagaccgttgatctcggttaaggtctttgtcgccagcaccttgattttatcgatgaggatgagagcgaggccagagatgtcggcagaggagccacgtgggtcggagttgagtagagagacacaaagtttgaagtctggtgcattgac

SEQ ID NO: 25 - DNA, 1600 bp, Camelina sativa
  misc_feature (383)..(383): n is a, c, g, or t
ctcaaaaattagcattaaaaattctgtaaatgaactttaataaatagtatatatttaattaaaaagcaatattgaaattttgaaaaccaaaaaaatgtatagtaattttgaaattcaaatcattgcaggaaattaaatacatagatggttttaggcataaatacactttccatatcatgatcacttgactaatattaatttggcatatttataatttcatagtaagatgttatttcagtgtggtcacaatattagacattatataatgtatatataatttatattagtgtttttgccaaatttgttcttggatactatagaaactaaaaagattaataacccaaactaaagaaatttaaaaacattcaaattaaattttgatnggacaatatcaatttggtggtatatactaaaataaaagtatattacctgaaaatatcagaaatgatatataggttttttatccttattaagagattttggtaaaggcacgccaccaattcaattatatatatactggtnncgggcagtacacagacaagacacacacacttataaataaacaaaaacgaaacctccatctttttacatataaagatcatcatccaacaagaagaagatgaagatggtcgtgatggttatgatgatgatgatgatgagtgaaggaagtatggtagatcaaacatgtaaacagacaccagacttcaatctctgtgtctctctactcaactccgacccacgtggctcttctgccgacacctctggcctcgctctcatcctcatcgataaaatcaaggtatttttcaattccttttctcatctagtttcttctatatagatattaccaattatctcagattattttcaagtcttattataagaatcaaatcttgactaaaggttttgtggttgttttttaaattatgatattttttctatattattagatgtaatatttaattttattctattctataactttgatctcttaaatttttataaaaaggctcataagtttcgttattctacgaaaaagtaattatcactaagacgtttttgtctataagactataagtaacacaaggggttgtttttgataaataagaagtttttgattacttttgtttagaacacatacctaagcctaagggtgttatttttttttgtgttttcatgtcgtagtaatattgttttcaatttcagtatagtgtatataaagctcgtttgtcgtttctatcccaccaattatgtagctttatttttccagaattatctgaattaaggggagagtttaactacaaataaaaaatgtgaggtaatttctgttgaaatataaacgtatggggttatcttataaatttttttttgtaggttctggcgacaaagaccttaaacgaaatcaacggtctatataaaaagagaccggaactaaaacaggctttagaccaatgtagtcgaagatacaaaacgatcttaaatgctgatgttcccgaagccatcgaagctatctctaaaggagtccctaaatttggcgaagatggtgtgatcgacgccggggtagaagcttctgtttgtgaagaagggtttcaagggaaatctc

SEQ ID NO: 26 - DNA, 1116 bp, Camelina sativa
tacgatggactccagagcggccgcggcgagacggtgaatgaactaatgtgtatatatatgtatgacttactttcgaataatgaactaatgtgtatgtatgacttactttcgaatgaagaaagttagaaagaatacaaattgattcttatttcagttgttcacatgtaaacacgttatatggcatcttgacaaaaagaaatatcacttaattcacattgagaattcttttgttttcatataggactattatatatagcaacaatatgtatcctgtaaatttgaatcccaattgtaacagccatatataatattagcataactattggactaaatgtcatggttaacgtagttaatgtgctattgtaattaattgtcataccacgtaaaaatcaataaaaggtactaaaatcatttcatattttgcaactacaaatgataaacaaaagtagtatttatttttatatatattttaaaatacgtaatatcaagaaactgcttaaaatataagacaagaatcctctttcttccatctctatctctctccgtagacagtttgctcaagcccctcttcttgaaatggcttcttctcttatcttcctcctcctcatctttaccctatcctttccatcctcaaccctaatctcagccaaatccaacgcgacaataatcgaatcaacttgcaaaaccacgaacaactacaaattctgtgtctcggctctcaaatccgacccaagaagtcccacagccgacacaaaaggtctcgcagccattatgatcggcgttggtatgacaaacgccacttccaccgcaacttacatcgccggaaacctaacatccgctgcaaacgacgtcgtccttaaaaaggtgttacaagattgctccgagaagtatgctctcgccgctgattctctccgtcaaacaattcaatatcttgataatgaagcttatgactatgcttccatgcatgtgctggcggcggaggattatcctaatgtttgccgcaatattttccgccgagctaaggggctgtcttatccggtggagattcgtcggcgtgaacagagtctgagacgtatctgtggtgttgtctcagggattcttgatcgtcttgttgaa

SEQ ID NO: 27 - DNA, 608 bp, Camelina sativa
gttaaaaattctttaaatgaactttaataaatagtatatatttaattaaaaagcaatattgaaattttgaaaaccaaaaaaatgtatagtaattttgaaattcaaatcattgcaggaaattaaatacatatatggttttaggcataaatacactttccatatcatgatcacttgactaatattaatttggcatatttataatttcatagtaagatcttatttcagtctggtcataatattagacattatataatgtatatataatttatattagtgtttttgccaaatttgttcttggatactatagaaactaaaaagattaataacccaaactaaagaaatctaaaaacattcaaattaaattttgattggacaatatcaatttggtggtatatactaaaataaaagtatattacctgaaaatatcagaaatgatatatagcttttttatccttattaagagattttggtaaaggcacaccaccaattcaattatatatatactggagacgggcactacacagacaagacacacacacttataaataaacaaaaagcgaaacctccatctttttacatataaagatcatcatccaacaagaagaagg

SEQ ID NO: 28 - DNA, 541 bp, Camelina sativa
aatgaactaatgtgtatatatatgtatgacttactttcgaataatgaactaatgtgtatgtatgacttactttcgaatgaagaaagttagaaagaatacaaattgattcttatttcagttgttcacatgtaaacacgttatatggcatcttgacaaaaagaaatatcacttaattcacattgagaattcttttgttttcatataggactattatatatagcaacaatatgtatcctgtaaatttgaatcccaattgtaacagccatatataatattagcataactattggactaaatgtcatggttaacgtagttaatgtgctattgtaattaattgtcataccacgtaaaaatcaataaaaggtactaaaatcatttcatattttgcaactacaaatgataaacaaaagtagtatttatttttatatatattttaaaatacgtaatatcaagaaactgcttaaaatataagacaagaatcctctttcttccatctctatctctctccgtagacagtttgctcaagcccctcttcttgaag

SEQ ID NO: 29 - protein, 57 aa, Nicotiana tabacum
MASSVLSSAAVATRSNVAQANMVAPFTGLKSAASFPVSRKQNLDITSIASNGGRVQC

SEQ ID NO: 30 - protein, 25 aa, Saccharomyces cerevisiae
MLSLRQSIRFFKPATRTLCSSRYLL

SEQ ID NO: 31 - protein, 78 aa, Arabidopsis thaliana
MYLTASSSASSSIIRAASSRSSSLFSFRSVLSPSVSSTSPSSLLARRSFGTISPAFRRWSHSFHSKPSPFRFTSQIRA

SEQ ID NO: 32 - protein, 19 aa, Saccharomyces cerevisiae
MLSARSAIKRPIVRGLATV

SEQ ID NO: 33 - protein, 26 aa, Arabidopsis thaliana
MRILPKSGGGALCLLFVFALCSVAHS

SEQ ID NO: 34 - protein, 85 aa, Arabidopsis thaliana
MLRTVSCLASRSSSSLFFRFFRQFPRSYMSLTSSTAALRVPSRNLRRISSPSVAGRRLLLRRGLRIPSAAVRSVNGQFSRLSVRA

SEQ ID NO: 35 - protein, 35 aa, Chlamydomonas reinhardtii
MALVARPVLSARVAASRPRVAARKAVRVSAKYGEN

SEQ ID NO: 36 - protein, 29 aa, Chlamydomonas reinhardtii
MQALSSRVNIAAKPQRAQRLVVRAEEVKA

SEQ ID NO: 37 - protein, 35 aa, Chlamydomonas reinhardtii
MQTLASRPSLRASARVAPRRAPRVAVVTKAALDPQ

SEQ ID NO: 38 - protein, 31 aa, Chlamydomonas reinhardtii
MQALATRPSAIRPTKAARRSSVVVRADGFIG

SEQ ID NO: 39 - protein, 51 aa, Chlamydomonas reinhardtii
MAFALASRKALQVTCKATGKKTAAKAAAPKSSGVEFYGPNRAKWLGPYSEN

SEQ ID NO: 40 - protein, 50 aa, Chlamydomonas reinhardtii
MAAVIAKSSVSAAVARPARSSVRPMAALKPAVKAAPVAAPAQANQMMVWT

SEQ ID NO: 41 - protein, 40 aa, Chlamydomonas reinhardtii
MAAMLASKQGAFMGRSSFAPAPKGVASRGSLQVVAGLKEV

SEQ ID NO: 42 - protein, 55 aa, Arabidopsis thaliana
MASSMLSSATMVASPAQATMVAPFNGLKSSAAFPATRKANNDITSITSNGGRVNC

SEQ ID NO: 43 - protein, 62 aa, Arabidopsis thaliana
MASSSFSVTSPAAAASVYAVTQTSSHFPIQNRSRRVSFRLSAKPKLRFLSKPSRSSYPVVKA

SEQ ID NO: 44 - protein, 4 aa, Arabidopsis thaliana
CVVQ

SEQ ID NO: 45 - DNA, 1755 bp, Lycopersicon pennellii
atggaattatttatgaaaaactcttctctttggggtttaaaattttatttattttgcctatttatagttttattaaacattaatagggtatttgcttctcataatatttttttggacttgcaatcttcaagtgctattagtgtcaagaatgttcatagaactcgttttcattttcaacctcctaaacattggattaatgaccctaatgcaccaatgtattataatggagtgtatcatttattctatcaatacaatccaaaaggatcagtatggggcaatattatttgggctcattcagtctcaaaagacttgataaattggatccatttagaacctgcaatttatccatccaaaaaatttgacaagtatggtacttggtctggatcatcaactattttacctaataacaaacctgttatcatatacaccggagtagtagattcgtataataatcaagtccagaactatgccatcccggctaacttatctgatccatttcttcgtaaatggatcaaacctaacaacaacccgttgatcgtccctgataatagtatcaatagaactgagtttcgcgatccaactacagcttggatgggccaagatgggctttggaggattttaataggaagtatgagaaaacatagagggatggcattgttgtatagaagtagagattttatgaaatggatcaaagcccaacatccacttcattcatctactaatactggaaattgggaatgtcctgattttttccctgtatcattaaatagtaccaatggtttagatgtatcgtatcgcggaaaaaatgttaaatatgtccttaagaatagtcttgatgttgctaggtttgattattacactattggcatgtatcacaccaaaatagataggtacattccgaataacaattcaattgatggatggaagggattgagaatcgactatggtaatttctatgcatcaaagacattctatgatcctagcagaaatcgaagggttatttggggttggtcaaatgaatcggatgtattacctgacgatgatattaagaaaggatgggctggaattcaaggtattccgcgacaagtatggctagaccttagtggtaaacaattagttcaatggcctattgaagaattagaaaccctaaggaagcaaaaggtccaattgaacaacaagaagttgagcaagggagaaatgtttgaagttaaaggaatctcagcatcacaggctgatgttgaagtdttattctcattttcaagtttaaacaaggccgaacaatttgatcctagatgggctgacctatatgcccaagacgtctgtgccattaagggttcgactatccaaggtgggcttggaccatttgggcttgcgacattagcttctaaaaacttagaagagtacacacctgttttcttccgagtgttcaaggctcaaaagaattataagattctcatgtgctcagatgctagaagatctaccatgagacaaaatgaagcaatgtataagccctcatttgctggatatgtagatgtagatttagtagacatgaagaagttatctcttaggagtttgattgatcactcagtagtggagagtttcggtgctggtggcaaaacatgcataacatcaagggtgtatccaagtttagcgatttatgataatgcacatttatttgtctttaacaatggctctgagacaatcacaattgagactctgaatgcttggagcatgggtgcatgtaagatgaactaa

SEQ ID NO: 46 - protein, 584 aa, Lycopersicon pennellii
MELFMKNSSLWGLKFYLFCLFIVLLNINRVFASHNIFLDLQSSSAISVKNVHRTRFHFQPPKHWINDPNAPMYYNGVYHLFYQYNPKGSVWGNIIWAHSVSKDLINWIHLEPAIYPSKKFDKYGTWSGSSTILPNNKPVIIYTGVVDSYNNQVQNYAIPANLSDPFLRKWIKPNNNPLIVPDNSINRTEFRDPTTAWMGQDGLWRILIGSMRKHRGMALLYRSRDFMKWIKAQHPLHSSTNTGNWECPDFFPVSLNSTNGLDVSYRGKNVKYVLKNSLDVARFDYYTIGMYHTKIDRYIPNNNSIDGWKGLRIDYGNFYASKTFYDPSRNRRVIWGWSNESDVLPDDDIKKGWAGIQGIPRQVWLDLSGKQLVQWPIEELETLRKQKVQLNNKKLSKGEMFEVKGISASQADVEVLFSFSSLNKAEQFDPRWADLYAQDVCAIKGSTIQGGLGPFGLATLASKNLEEYTPVFFRVFKAQKNYKILMCSDARRSTMRQNEAMYKPSFAGYVDVDLVDMKKLSLRSLIDHSVVESFGAGGKTCITSRVYPSLAIYDNAHLFVFNNGSETITIETLNAWSMGACKMN

SEQ ID NO: 47 - DNA, 1797 bp, Oryza sativa
atgggagttcttggtagtagggtcgcttgggcatggctggtccagctgctgctgctccagcagctcgccggagcgtcgcacgtcgtctacgacgacctcgagctgcaggcggctgctaccacagcggacggcgtgccgccgtccatcgtcgactctgagctccggactgggtatcacttccagccacccaagaactggatcaatgatccgaacgcgccgatgtactacaaggggtggtaccatctgttctaccagtacaaccccaagggcgccgtgtgggggaacatcgtgtgggcgcactcagtgtcacgtgacctcatcaactgggtggcgctcaagccggccatcgagcccagcatcagggccgacaagtacggctgctggtcggggtcggcgacgatgatggccgacgggacgccggtgatcatgtacaccggcgtcaaccgccccgacgtcaactaccaggtgcagaacgtggcgctgccgaggaacgggtcggacccgctgctgcgcgagtgggtgaagcccggccacaacccggtgatcgtgcccgagggcggcatcaacgcgacgcagttccgcgacccgaccaccgcgtggcgcggggccgacggccactggcggctgctcgtcggcagcctcgcggggcagtcccgcggcgtggcgtacgtgtaccggagcagggacttccggcggtggacgcgcgcggcgcagccgctgcactcggcgcccacggggatgtgggagtgcccggacttctacccggtcaccgcggacggccgccgcgagggcgtcgacacctcgtccgccgtcgtcgacgccgccgcctcggcgcgcgtcaagtacgtgctcaagaacagcctcgacctgcgccggtacgactactacaccgtcggaacgtacgaccggaaggccgagcggtacgtgccggacgaccccgccggcgacgagcaccacatccgctacgactacggcaacttctacgcctccaagacgttctacgacccggcgaagcgccgccgcatcctctggggatgggccaacgagtccgacaccgccgccgacgacgtggccaagggctgggccggaatccaggcgattccgaggaaagtgtggctggacccaagtgggaagcaactgttgcagtggccaatcgaggaggtcgagaggctgagagggaagtggccggtcattctcaaggacagggtggtcaagccaggggaacacgtcgaggtgaccgggctacaaactgcacaggctgacgtggaggtgagcttcgaggtggggagcctggaggcggcggagcggctggacccggcgatggcgtacgacgcgcagcggctgtgcagcgcgcggggcgccgacgcgaggggcggcgtggggccgttcggcctgtgggtgctcgcgtccgcggggctggaggagaagaccgccgtgttcttcagggtgttcaggccggcggcgcgcggcggcggcgccggcaagcccgtcgtgctcatgtgcaccgaccccaccaagtcatcgcgcaacccgaacatgtaccagccgacgtttgcagggttcgttgacacggacatcaccaacgggaagatatctctgaggagcctgatcgacaggtcggttgttgagagcttcggggctggaggaaaggcgtgcatcctgtcgagggtgtacccgtcgctggccatcggcaagaacgcgcgcctttacgttttcaataacgggaaggcggagatcaaggtgtcgcagctcaccgcgtgggagatgaagaagccggtcatgatgaatggagcctaa

SEQ ID NO: 48 - protein, 598 aa, Oryza sativa
MGVLGSRVAWAWLVQLLLLQQLAGASHVVYDDLELQAAATTADGVPPSIVDSELRTGYHFQPPKNWINDPNAPMYYKGWYHLFYQYNPKGAVWGNIVWAHSVSRDLINWVALKPAIEPSIRADKYGCWSGSATMMADGTPVIMYTGVNRPDVNYQVQNVALPRNGSDPLLREWVKPGHNPVIVPEGGINATQFRDPTTAWRGADGHWRLLVGSLAGQSRGVAYVYRSRDFRRWTRAAQPLHSAPTGMWECPDFYPVTADGRREGVDTSSAVVDAAASARVKYVLKNSLDLRRYDYYTVGTYDRKAERYVPDDPAGDEHHIRYDYGNFYASKTFYDPAKRRRILWGWANESDTAADDVAKGWAGIQAIPRKVWLDPSGKQLLQWPIEEVERLRGKWPVILKDRVVKPGEHVEVTGLQTAQADVEVSFEVGSLEAAERLDPAMAYDAQRLCSARGADARGGVGPFGLWVLASAGLEEKTAVFFRVFRPAARGGGAGKPVVLMCTDPTKSSRNPNMYQPTFAGFVDTDITNGKISLRSLIDRSVVESFGAGGKACILSRVYPSLAIGKNARLYVFNNGKAEIKVSQLTAWEMKKPVMMNGA

SEQ ID NO: 49 - DNA, 1085 bp, Camelina sativa
aaccggaatgtgggaatgtcctgattttttcccggtctcgacaaccggttcggacggtgttgagacgtcgtcgttcgttcaggatgaggttaagtacgtgcttaaagtgagtttgattgagacactacatgattattacacgattgggagttacgatcgtgagaaagatgtgtatgtaccggatcttgggtttgtgcaagacgaaacggctccgaggttagattacgggaaatactacgcgtcgaaaacgttttacgatgatgataagaaacgaaggatcttgtggggttgggttaatgaatcgtctccggctaaagatgatatcaagaagggttgggctggtcttcagtcatttccgaggaagatatggctagatgaatcgggaaaggaattattacaatggccgattgaagagattgagacattgcgtgggacacaagtcaactggcacaacaaaattcttgaagcaggatctactctccaagttcatggtgtcactgctgcacaggcagatgttgaggtgttattcaaggtaaatgaaattgagaaagcagatgtaattgatccgagttggaccgacccacaaaagatatgtagtcaagaagaatcgtcggttaagtccggtataggaccatttggtttgaaggttttggcatccaaggacatggaagagtacacgtcggtatacttcagaatcttcaagtcgaatgatactaataagaataccaagtacctggtgttgatgtgcagtaaccagaacagatcttcgttgaatgatgaaaatgataaatccgcatttggtgcttttgtggcgatagacccttctcaccaaacaatttctcttaggactttgattgatcactcgatagtggagagttatggtggaggaggcagaaaatgtataacctctagagtgtatccaaaattggcaattggagaaaatgcaaatctttttgccttcaacaaaggaactcaaagtgttgatgtcttaagcctaagtgcttggagcttgaagtctgctcaaatcaatgacgagtcgatttcaccttttatcgagcgtgaagatgcacactcacctaaacagt

SEQ ID NO: 50 - protein, 361 aa, Camelina sativa
TGMWECPDFFPVSTTGSDGVETSSFVQDEVKYVLKVSLIETLHDYYTIGSYDREKDVYVPDLGFVQDETAPRLDYGKYYASKTFYDDDKKRRILWGWVNESSPAKDDIKKGWAGLQSFPRKIWLDESGKELLQWPIEEIETLRGTQVNWHNKILEAGSTLQVHGVTAAQADVEVLFKVNEIEKADVIDPSWTDPQKICSQEESSVKSGIGPFGLKVLASKDMEEYTSVYFRIFKSNDTNKNTKYLVLMCSNQNRSSLNDENDKSAFGAFVAIDPSHQTISLRTLIDHSIVESYGGGGRKCITSRVYPKLAIGENANLFAFNKGTQSVDVLSLSAWSLKSAQINDESISPFIEREDAHSPKQ

SEQ ID NO: 51 - DNA, 1284 bp, Camelina sativa
tggtccggctccgtcacgattctccctaatggcaaacccgtacttctctacacaggcaacgaccgttacaaccgtcaggtccaaaacctagccatacccaaaaacctaaccgatccatatctccggcactggacaaaatctccggaaaatcccctcgtgacacctggcgacgccaaccacatcaactccaccgcgtttcgcgacccaaccaccgcgtggctaggccgtgatggtcgatggcgtataaccacgggaagccaagacggtcgccgagggttagcgattctacacacgagccgagatttcgtgaggtggaagcaatctccaaagcctctacattaccacgacggcactgggatgtgggagtgccctgattttttcccggtggcgagaactgattcacgcggcgtcgatacgacggcgtttaatgggaagatggtgaagcacgtgcttaaagtgagcttaacagatacgtttcatgattactacacgattggaacgtacgaccaagtgagagatgtctacgtaccggacaatggttttgttcaggatgaaacggctccgagatacgactacggcaagttttacgcctcaaagacgttttacgactcggtttaccaacggaggattttgtggggttgggttaacgagtcgtcgacggataaggacattgtcaatatgggttgggccggtttacaggctattcctaggaaaatatggcttgatgaatcaggaaagagtttggtgcaatggccagttaaagaaatagagaggttacgtacaacgcaagtcaagtggggcaacaaagttttgaaaggaggaggatcggtcatggaggttcatggagtcacagcttcacaagcagatgtggaggttttctttaaagtgagtggtttagacttagagaaagcagatgtgattgaaccaggttggaccgacccgcagttgatttgcagccagaagaatgcatcgttggttaagtccggtttaggtccatttggtttgatggtactggcttccaagaacttggaagagtacacatcagtgtatctcagaatcttcaaagctcgtgagaatagtaaggagcatgtggtggtcatgtgcaatgaccaaagcagatcaagtttagagaaaggaaatgataaaacaacgtatggtgcttttgtggatgtctctccttatcaaacaatctctctcaggactttgattgataattcaatagtggagagctttggtgggaaagggaaagcatgtattacctcaagagtttatccaaaattggca

SEQ ID NO: 52 - protein, 428 aa, Camelina sativa
WSGSVTILPNGKPVLLYTGNDRYNRQVQNLAIPKNLTDPYLRHWTKSPENPLVTPGDANHINSTAFRDPTTAWLGRDGRWRITTGSQDGRRGLAILHTSRDFVRWKQSPKPLHYHDGTGMWECPDFFPVARTDSRGVDTTAFNGKMVKHVLKVSLTDTFHDYYTIGTYDQVRDVYVPDNGFVQDETAPRYDYGKFYASKTFYDSVYQRRILWGWVNESSTDKDIVNMGWAGLQAIPRKIWLDESGKSLVQWPVKEIERLRTTQVKWGNKVLKGGGSVMEVHGVTASQADVEVFFKVSGLDLEKADVIEPGWTDPQLICSQKNASLVKSGLGPFGLMVLASKNLEEYTSVYLRIFKARENSKEHVVVMCNDQSRSSLEKGNDKTTYGAFVDVSPYQTISLRTLIDNSIVESFGGKGKACITSRVYPKLA

SEQ ID NO: 53 - DNA, 1409 bp, Camelina sativa
tacaaaggattctaccatctgttctaccaacacaaccctttggctccatattttggggacattatggtatggggacactctgtttcacaagatttggtcaactggatccaactagaaacagccatttatccctcagatccctctgacatcaacagttgctggtcaggatccgccacgatcctccctgatggcaaacctgtaatgctgtacacaggaagcgacaccaataaacaccaggtgacagttcttgcggaacctaaggatgcgtctgaccctttgcttcgtgagtgggtaaaggctaaaggcaaccctgtgatggttccacctagtaacgtccccgttgatggtttccgtgacccaactacggcgtggcaaggtcaagatgggagatggagagttcttgttggagctacggagaaagatagtgataaagggatggcgattttgtaccacagtgatgattttttccagtggacaaagtatccggtggctttacttgaatcacaaatcaccggaatgtgggaatgtccagactttttccccgtgtcagttacgggaagagagggtttagatacttcggtgaacaattctagtgtgaggcatgtgttgaaggcgagttttggaggcagtgattgctatgtcattggtacatatacttccgagacagaaatcttttccgcagattccgagttcactaacacagctgatgatttgagatatgattacggaaatctatacgcgtccaaggccttctttgatagcgctaagaataggaggatctcatggggatggattatggagaaagatagcaatgaagatgatattgtgaaaggatgggctggaattatgggtctcccaagggagatttggctggaaagaagtggaaagaagttgatgcaatggccagtcgaggaaattaacaatctccgagccaaaaatgttagcttgtacaacaaacaacttgaaagcggctctgttcttgaaatctctggcatcactgcatcacaagccgacatagaagtagcttttgatttacgtgatctagaaaacgatcccgagattttggattcagaggaagttgatcaagcgacttttacagctggttacagcgcatcggttagaggcatttacggtccttttggattgttagcattggctagcaatgatttagcagagcacactgcaatcttctttagagttcttcgtcgtgggaacggatattcggttgtgatgtgcagcgatgagagcaagtcttcattgagagacaacattgagaaagctacatttggaacagtccttgatatcgacccaagacatgaaaagatctcgttaagatgtttgattgatcactcggttatagagagctttgggggagaaggaagaagtgtgataacgtctagggtttatcc

SEQ ID NO: 54 - protein, 469 aa, Camelina sativa
YKGFYHLFYQHNPLAPYFGDIMVWGHSVSQDLVNWIQLETAIYPSDPSDINSCWSGSATILPDGKPVMLYTGSDTNKHQVTVLAEPKDASDPLLREWVKAKGNPVMVPPSNVPVDGFRDPTTAWQGQDGRWRVLVGATEKDSDKGMAILYHSDDFFQWTKYPVALLESQITGMWECPDFFPVSVTGREGLDTSVNNSSVRHVLKASFGGSDCYVIGTYTSETEIFSADSEFTNTADDLRYDYGNLYASKAFFDSAKNRRISWGWIMEKDSNEDDIVKGWAGIMGLPREIWLERSGKKLMQWPVEEINNLRAKNVSLYNKQLESGSVLEISGITASQADIEVAFDLRDLENDPEILDSEEVDQATFTAGYSASVRGIYGPFGLLALASNDLAEHTAIFFRVLRRGNGYSVVMCSDESKSSLRDNIEKATFGTVLDIDPRHEKISLRCLIDHSVIESFGGEGRSVITSRVY